SEQUENCE LISTING (2 sequences)

SEQ ID NO: 1 - RNA, 19 nt, Artificial Sequence
  Synthetic
  misc_feature (1)..(1): n is guanosine triphosphate
ncaugcgaccucuguuuga

SEQ ID NO: 2 - RNA, 19 nt, Artificial Sequence
  Synthetic
ucaaacagaggucgcaugc

(19) United States
(12) Patent Application Publication
(10) Pub. No.: US 2020/0108151 A1
(43) Pub. Date: Apr. 9, 2020

(54) ANTIBODY ADJUVANT CONJUGATES

(71) Applicant: Bolt Biotherapeutics, Inc., Redwood City, CA (US)

(72) Inventors: David Y. Jackson, Belmont, CA (US); Michael Nathaniel Alonso, Redwood City, CA (US); Arthur Lee, Redwood City, CA (US)

(73) Assignee: Bolt Biotherapeutics, Inc., Redwood City, CA (US)

(21) Appl. No.: 16/723,276

(22) Filed: Dec. 20, 2019

Related U.S. Application Data
(63) Continuation of application No. 16/129,118, filed on Sep. 12, 2018, now abandoned.
Application No. 16/129,118 is a continuation of application No. PCT/US2017/066220, filed on Dec. 13, 2017.
(60) Provisional application No. 62/433,742, filed on Dec. 13, 2016.

(51) Int. Cl. A61K 47/55; A61K 47/68; A61P 35/00

(52) U.S. Cl. CPC A61K 47/55; A61K 47/6803; A61K 47/6849; A61P 35/00; A61K 47/6851

(57) ABSTRACT

The invention provides an immunoconjugate comprising an antibody construct which includes an antigen binding domain and an Fc domain, an adjuvant moiety, and a linker, wherein each adjuvant moiety is covalently bonded to the antibody via the linker which comprises an ethylene glycol group or glycine residue. Methods for treating cancer with the immunoconjugates of the invention are also described.

### CROSS-REFERENCE TO RELATED APPLICATIONS

This patent application is a continuation of copending U.S. patent application Ser. No. 16/129,118, filed Sep. 12, 2018, which is a continuation of International Patent Application No. PCT/US2017/066220, filed Dec. 13, 2017, which claims the benefit of U.S. Provisional Patent Application No. 62/433,742, filed Dec. 13, 2016, each of which is incorporated by reference in its entirety herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 762 Byte ASCII (Text) file named “747042_ST25.txt,” created on Dec. 17, 2019.

### BACKGROUND OF THE INVENTION

It is now well appreciated that tumor growth necessitates the acquisition of mutations that facilitate immune evasion. Even so, tumorigenesis results in the accumulation of mutated antigens, or neoantigens, that are readily recognized by the host immune system following ex vivo stimulation. Why and how the immune system fails to recognize neoantigens are beginning to be elucidated. Groundbreaking studies by Carmi et al. (Nature, 521: 99-104 (2015)) have indicated that immune ignorance can be overcome by delivering neoantigens to activated dendritic cells via antibody-tumor immune complexes. In these studies, simultaneous delivery of tumor binding antibodies and dendritic cell adjuvants via intratumoral injections resulted in robust anti-tumor immunity. New compositions and methods for the delivery of antibodies and dendritic cell adjuvants are needed in order to reach inaccessible tumors and to expand treatment options for cancer patients and other subjects. The invention addresses this and other needs.

### BRIEF SUMMARY OF THE INVENTION

In a first aspect, the invention provides an immunoconjugate comprising (a) an antibody construct comprising (i) an antigen binding domain and (ii) an Fc domain, (b) an adjuvant moiety, and (c) a linker comprising an ethylene glycol group or a glycine residue, wherein each adjuvant moiety is covalently bonded to the antibody construct via the linker.

In another aspect, the invention provides an immunoconjugate having a structure according to Formula II:

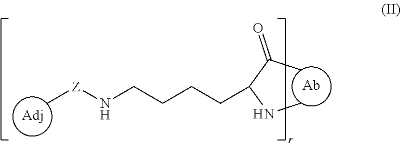

wherein

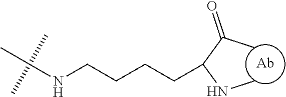

is an antibody with residue

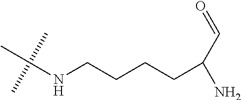

representing a lysine residue of the antibody, wherein “ [symbol] ” represents a point of attachment to Z; Adj is an adjuvant; subscript r is an integer from 1 to 10; and Z is a divalent linking moiety having an ethylene glycol group or a glycine residue.

In a further aspect, the invention provides a composition comprising a plurality of immunoconjugates of the invention.

In another aspect, the invention provides methods of treating cancer comprising administering a therapeutically effective amount of an immunoconjugate according to the invention, or a composition comprising an immunoconjugate of the invention, to a subject in need thereof.

### BRIEF DESCRIPTION OF THE DRAWINGS

FIG. 1 shows the structure of adjuvant CL264, wherein the circle indicates a position on the adjuvant where it can be conjugated to the linker, specifically, the terminal carboxylic acid of the adjuvant.

FIG. 2 shows the structure of adjuvant CL401, wherein the circle indicates a position on the adjuvant where it can be conjugated to the linker, specifically, the primary amine of the adjuvant.

FIG. 3 shows the structure of adjuvant CL413, wherein the circle indicates a position on the adjuvant where it can be conjugated to the linker, specifically, the first lysine residue of the adjuvant.

FIG. 4 shows the structure of adjuvant CL413, wherein the circle indicates a position on the adjuvant where it can be conjugated to the linker, specifically, the second lysine residue of the adjuvant.

FIG. 5 shows the structure of adjuvant CL413, wherein the circle indicates a position on the adjuvant where it can be conjugated to the linker, specifically, the third lysine residue of the adjuvant.

FIG. 6 shows the structure of adjuvant CL413, wherein the circle indicates a position on the adjuvant where it can be conjugated to the linker, specifically, the fourth lysine residue of the adjuvant.

FIG. 7 shows the structure of adjuvant CL413, wherein the circle indicates a position on the adjuvant where it can be conjugated to the linker, specifically, the primary amine of the adjuvant.

FIG. 8 shows the structure of adjuvant CL419, wherein the circles indicate positions on the adjuvant where it can be conjugated to the linker, specifically, the amines of the adjuvant (terminal amine in the top part of FIG. 8 and secondary amine in the bottom part of FIG. 8).

FIG. 9 shows the structure of adjuvant CL553, wherein the circle indicates a position on the adjuvant where it can be conjugated to the linker, specifically, a secondary amine of the adjuvant.

FIG. 10 shows the structure of adjuvant CL553, wherein the circle indicates a position on the adjuvant where it can be conjugated to the linker, specifically, another secondary amine of the adjuvant.

FIG. 11 shows the structure of adjuvant CL553, wherein the circle indicates a position on the adjuvant where it can be conjugated to the linker, specifically, a primary amine of the adjuvant.

FIG. 12 shows the structure of adjuvant CL553, wherein the circle indicates a position on the adjuvant where it can be conjugated to the linker, specifically, an amide of the adjuvant.

FIG. 13 shows the structure of adjuvant CL572, wherein the circles indicate positions on the adjuvant where it can be conjugated to the linker, specifically, the primary amine (top part of FIG. 13) and the carbonyl (bottom part of FIG. 13).

FIG. 14 shows the structure of adjuvant Pam2CSK4, wherein the circle indicates a position on the adjuvant where it can be conjugated to the linker, specifically, the terminal carboxylic acid of the adjuvant.

FIG. 15 shows the structure of adjuvant Pam2CSK4, wherein the circle indicates a position on the adjuvant where it can be conjugated to the linker, specifically, the terminal thiol of the adjuvant.

FIG. 16 shows the structure of adjuvant Pam2CSK4, wherein the circle indicates a position on the adjuvant where it can be conjugated to the linker, specifically, the second lysine residue of the adjuvant.

FIG. 17 shows the structure of adjuvant Pam2CSK4, wherein the circle indicates a position on the adjuvant where it can be conjugated to the linker, specifically, the third lysine residue of the adjuvant.

FIG. 18 shows the structure of adjuvant Pam2CSK4, wherein the circle indicates a position on the adjuvant where it can be conjugated to the linker, specifically, the terminal lysine residue of the adjuvant.

FIG. 19 shows the structure of adjuvant Pam3CSK4, wherein the circle indicates a position on the adjuvant where it can be conjugated to the linker, specifically, the terminal carboxylic acid of the adjuvant.

FIG. 20 shows the structure of adjuvant Pam3CSK4, wherein the circle indicates a position on the adjuvant where it can be conjugated to the linker, specifically, the terminal thiol of the adjuvant.

FIG. 21 shows the structure of adjuvant Pam3CSK4, wherein the circle indicates a position on the adjuvant where it can be conjugated to the linker, specifically, the second lysine residue of the adjuvant.

FIG. 22 shows the structure of adjuvant Pam3CSK4, wherein the circle indicates a position on the adjuvant where it can be conjugated to the linker, specifically, the third lysine residue of the adjuvant.

FIG. 23 shows the structure of adjuvant Pam3CSK4, wherein the circle indicates a position on the adjuvant where it can be conjugated to the linker, specifically, the terminal lysine residue of the adjuvant.

### DETAILED DESCRIPTION OF THE INVENTION

### General

The invention provides antibody-adjuvant immunoconjugates having a number of advantages including antibodies that promote antibody-dependent cellular cytotoxicity, antibody-dependent cellular phagocytosis and antibodies that block the actions of cancer produced proteins that act as immune checkpoint molecules, adjuvants that promote dendritic cell activation and T cell proliferation, and covalent linkages between antibody and adjuvant that promote anti-tumor efficacy. For example, human monocytes undergo DC differentiation following overnight stimulation with antibody-adjuvant immunoconjugates of the invention, whereas DC differentiation protocols with known stimulants (e.g., GM-CSF and IL-4) require much longer periods. Antibody-adjuvant immunoconjugate-activated cells also express several fold higher amounts of co-stimulatory molecules and inflammatory cytokines than achievable with known stimulants. Antibody-adjuvant immunoconjugate-activated cells express higher amounts (e.g., in some cases several fold higher amounts) of co-stimulatory molecules and inflammatory cytokines than is achievable with known stimulants.

Antibody-adjuvant immunoconjugates which are covalently attached, i.e., wherein the antibody is covalently bonded to the linker which is covalently bonded to the adjuvant, are quantitatively and qualitatively more effective at eliciting immune activation than non-covalently attached antibody-adjuvant immunoconjugates. Further, antibody-adjuvant immunoconjugates linked according to the invention are much more effective than other known immunoconjugates. Systemic administration of the adjuvant-antibody conjugates allows for the simultaneous targeting of the primary tumor and associated metastases without the need for intra-tumoral injections and surgical resection.

### Definitions

As used herein, the term “immunoconjugate” refers to an antibody construct, or antibody, that is covalently bonded to a non-naturally occurring chemical moiety as described herein. The terms “immunoconjugate” and “antibody-adjuvant immunoconjugate” are used interchangeably herein.

As used herein, the phrase “antibody construct” refers to polypeptide comprising an antigen binding domain and an Fc domain. An antibody construct can comprise or be an antibody.

As used herein, the phrase “antigen binding domain” refers to a protein, or a portion of a protein, that specifically binds a specified antigen (e.g., a paratope), for example, that portion of an antigen-binding protein that contains the amino acid residues that interact with an antigen and confer on the antigen-binding protein its specificity and affinity for the antigen.

As used herein, the phrase “Fc domain” refers to the fragment crystallizable region, or the tail region of an antibody. The Fc domain interacts with Fc receptors on cell surfaces.

As used herein, the phrase “targeting binding domain” refers to a protein, or a portion of a protein, that specifically binds a second antigen that is distinct from the antigen bound by the antigen binding domain of the immunoconjugates. The targeting binding domain can be conjugated to the antibody construct at a C-terminal end of the Fc domain.

As used herein, the term “antibody” refers to a polypeptide comprising an antigen binding region (including the complementarity determining region (CDRs)) from an immunoglobulin gene or fragments thereof that specifically binds and recognizes an antigen. The recognized immunoglobulin genes include the kappa, lambda, alpha, gamma, delta, epsilon, and mu constant region genes, as well as numerous immunoglobulin variable region genes.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. Light chains are classified as either kappa or lambda. Heavy chains are classified as gamma, mu, alpha, delta, or epsilon, which in turn define the immunoglobulin classes IgG, IgM, IgA, IgD, and IgE, respectively.

IgG antibodies are large molecules of about 150 kDa composed of four peptide chains. IgG antibodies contain two identical class γ heavy chains of about 50 kDa and two identical light chains of about 25 kDa, forming a tetrameric quaternary structure. The two heavy chains are linked to each other and to a light chain each by disulfide bonds. The resulting tetramer has two identical halves, which together form the Y-like shape. Each end of the fork contains an identical antigen binding site. There are four IgG subclasses (IgG1, 2, 3, and 4) in humans, named in order of their abundance in serum (IgG1 being the most abundant). Typically, the antigen-binding region of an antibody will be most critical in specificity and affinity of binding.

Dimeric IgA antibodies are around 320 kDa. IgA has two subclasses (IgA1 and IgA2) and can be produced as a monomeric as well as a dimeric form. The IgA dimeric form (secretory or sIgA) is the most abundant.

Antibodies can exist, for examples, as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see, e.g., Fundamental Immunology (Paul ed., 7th ed. 2012). While various antibody fragments are defined in terms of the digestion of an intact antibody, such fragments may be synthesized de novo either chemically or by using recombinant DNA methodology. Thus, the term antibody, as used herein, also includes antibody fragments produced by the modification of whole antibodies, synthesized de novo using recombinant DNA methodologies (e.g., single chain Fv), or identified using phage display libraries (see, e.g., McCafferty et al., Nature, 348: 552-554 (1990)).

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibody fragment” and all grammatical variants thereof, as used herein are defined as a portion of an intact antibody comprising the antigen binding site or variable region of the intact antibody, wherein the portion is free of the constant heavy chain domains (i.e., CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; (2) single chain polypeptides containing only one light chain variable domain, or a fragment thereof that contains the three CDRs of the light chain variable domain, without an associated heavy chain moiety; (3) single chain polypeptides containing only one heavy chain variable region, or a fragment thereof containing the three CDRs of the heavy chain variable region, without an associated light chain moiety; (4) nanobodies comprising single Ig domains from non-human species or other specific single-domain binding modules; and (5) multispecific or multivalent structures formed from antibody fragments. In an antibody fragment comprising one or more heavy chains, the heavy chain(s) can contain any constant domain sequence (e.g., CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used herein, the term “biosimilar” in reference to a biological product means that the biological product is highly similar to the reference product notwithstanding minor differences in clinically inactive components, and there are no clinically meaningful differences between the biological product and the reference product in terms of the safety, purity, and potency of the product.

As used herein, the term “epitope” means any antigenic determinant on an antigen to which the antigen-binding site, also referred to as the paratope, of an antibody binds. Epitopic determinants usually consist of chemically active surface groupings of molecules such as amino acids or sugar side chains and usually have specific three-dimensional structural characteristics, as well as specific charge characteristics.

The terms “polypeptide,” “peptide,” and “protein” are used interchangeably herein to refer to a polymer of amino acid residues. The terms also apply to amino acid polymers in which one or more amino acid residues are artificial chemical mimetics of a corresponding naturally occurring amino acids, as well as to naturally occurring amino acid polymers and non-naturally occurring amino acid polymer.

As used herein, the term “adjuvant” refers to a substance capable of eliciting an immune response in a subject exposed to the adjuvant.

As used herein, the term “adjuvant moiety” refers to an adjuvant that is covalently bonded to an antibody as described herein. The adjuvant moiety can elicit the immune response while bonded to the antibody or after cleavage (e.g., enzymatic cleavage) from the antibody following administration of an immunoconjugate to the subject.

As used herein, the terms “Pattern recognition receptor” and “PRR” refer to any member of a class of conserved mammalian proteins which recognize pathogen-associated molecular patterns (PAMPs) or damage-associated molecular patterns (DAMPs), and act as key signaling elements in innate immunity. Pattern recognition receptors are divided into membrane-bound PRRs, cytoplasmic PRRs, and secreted PRRs. Examples of membrane-bound PRRs include Toll-like receptors (TLRs) and C-type lectin receptors (CLRs). Examples of cytoplasmic PRRs include NOD-like receptors (NLRB) and Rig-I-like receptors (RLRs).

As used herein, the terms “Toll-like receptor” and “TLR” refer to any member of a family of highly-conserved mammalian proteins which recognize pathogen-associated molecular patterns and act as key signaling elements in innate immunity. TLR polypeptides share a characteristic structure that includes an extracellular domain that has leucine-rich repeats, a transmembrane domain, and an intracellular domain that is involved in TLR signaling.

The terms “Toll-like receptor 1” and “TLR1” refer to nucleic acids or polypeptides sharing at least 70%; 80%, 90%, 95%, 96%, 97%, 98%, 99%, or more sequence identity to a publicly-available TLR1 sequence, e.g., GenBank accession number AAY85643 for human TLR1 polypeptide, or GenBank accession number AAG37302 for murine TLR1 polypeptide.

The terms “Toll-like receptor 2” and “TLR2” refer to nucleic acids or polypeptides sharing at least 70%; 80%, 90%, 95%, 96%, 97%, 98%, 99%, or more sequence identity to a publicly-available TLR2 sequence, e.g., GenBank accession number AAY85648 for human TLR2 polypeptide, or GenBank accession number AAD49335 for murine TLR2 polypeptide.

The terms “Toll-like receptor 3” and “TLR3” refer to nucleic acids or polypeptides sharing at least 70%; 80%, 90%, 95%, 96%, 97%, 98%, 99%, or more sequence identity to a publicly-available TLR3 sequence, e.g., GenBank accession number AAC34134 for human TLR3 polypeptide, or GenBank accession number AAK26117 for murine TLR3 polypeptide.

The terms “Toll-like receptor 4” and “TLR4” refer to nucleic acids or polypeptides sharing at least 70%; 80%, 90%, 95%, 96%, 97%, 98%, 99%, or more sequence identity to a publicly-available TLR4 sequence, e.g., GenBank accession number AAY82270 for human TLR4 polypeptide, or GenBank accession number AAD29272 for murine TLR4 polypeptide.

The terms “Toll-like receptor 5” and “TLR5” refer to nucleic acids or polypeptides sharing at least 70%; 80%, 90%, 95%, 96%, 97%, 98%, 99%, or more sequence identity to a publicly-available TLR5 sequence, e.g., GenBank accession number ACM69034 for human TLR5 polypeptide, or GenBank accession number AAF65625 for murine TLR5 polypeptide.

The terms “Toll-like receptor 6” and “TLR6” refer to nucleic acids or polypeptides sharing at least 70%; 80%, 90%, 95%, 96%, 97%, 98%, 99%, or more sequence identity to a publicly-available TLR6 sequence, e.g., GenBank accession number ABY67133 for human TLR6 polypeptide, or GenBank accession number AAG38563 for murine TLR6 polypeptide.

The terms “Toll-like receptor 7” and “TLR7” refer to nucleic acids or polypeptides sharing at least 70%; 80%, 90%, 95%, 96%, 97%, 98%, 99%, or more sequence identity to a publicly-available TLR7 sequence, e.g., GenBank accession number AAZ99026 for human TLR7 polypeptide, or GenBank accession number AAK62676 for murine TLR7 polypeptide.

The terms “Toll-like receptor 8” and “TLR8” refer to nucleic acids or polypeptides sharing at least 70%; 80%, 90%, 95%, 96%, 97%, 98%, 99%, or more sequence identity to a publicly-available TLR8 sequence, e.g., GenBank accession number AAZ95441 for human TLR8 polypeptide, or GenBank accession number AAK62677 for murine TLR8 polypeptide.

The terms “Toll-like receptor 7/8” and “TLR7/8” refer to nucleic acids or polypeptides that are both TLR7 agonists and TLR8 agonists.

The terms “Toll-like receptor 9” and “TLR9” refer to nucleic acids or polypeptides sharing at least 70%; 80%, 90%, 95%, 96%, 97%, 98%, 99%, or more sequence identity to a publicly-available TLR9 sequence, e.g., GenBank accession number AAF78037 for human TLR9 polypeptide, or GenBank accession number AAK28488 for murine TLR9 polypeptide.

The terms “Toll-like receptor 10” and “TLR10” refer to nucleic acids or polypeptides sharing at least 70%; 80%, 90%, 95%, 96%, 97%, 98%, 99%, or more sequence identity to a publicly-available TLR10 sequence, e.g., GenBank accession number AAK26744 for human TLR10 polypeptide.

The terms “Toll-like receptor 11” and “TLR11” refer to nucleic acids or polypeptides sharing at least 70%; 80%, 90%, 95%, 96%, 97%, 98%, 99%, or more sequence identity to a publicly-available TLR11 sequence, e.g., GenBank accession number AAS83531 for murine TLR11 polypeptide.

A “TLR agonist” is a substance that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as NK-κB, in the association of certain components (such as IRAK) with other proteins or intracellular structures, or in the biochemical activity of components such as kinases (such as MAPK).

As used herein, the term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, γ-carboxyglutamate, and O-phosphoserine. Naturally-occurring a-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to herein by either the commonly known three letter symbols or by the one-letter symbols recommended by the IUPAC-IUB Biochemical Nomenclature Commission.

As used herein, the term “immune checkpoint inhibitors” refers to any modulator that inhibits the activity of the immune checkpoint molecule. Immune checkpoint inhibitors can include, but are not limited to, immune checkpoint molecule binding proteins, small molecule inhibitors, antibodies, antibody-derivatives (including Fc fusions, Fab fragments and scFvs), antibody-drug conjugates, antisense oligonucleotides, siRNA, aptamers, peptides and peptide mimetics.

As used herein, the term “linking moiety” refers to a functional group that covalently bonds two or more moieties in a compound or material. For example, the linking moiety can serve to covalently bond an adjuvant moiety to an antibody in an immunoconjugate.

Useful bonds for connecting linking moieties to proteins and other materials include, but are not limited to, amides, amines, esters, carbamates, ureas, thioethers, thiocarbamates, thiocarbonates, and thioureas. A “divalent” linking moiety contains two points of attachment for linking two functional groups; polyvalent linking moieties can have additional points of attachment for linking further functional groups. For example, divalent linking moieties include divalent polymer moieties such as divalent poly(ethylene glycol), divalent poly(propylene glycol), and divalent poly(vinyl alcohol).

As used herein, when the term “optionally present” is used to refer to a chemical structure (e.g., “R” or “Q”), if that chemical structure is not present, the bond originally made to the chemical structure is made directly to the adjacent atom.

As used herein, the term “linker” refers to a functional group that covalently bonds two or more moieties in a compound or material. For example, the linker can serve to covalently bond an adjuvant moiety to an antibody construct in an immunoconjugate.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 30 carbons atoms, such as, but not limited to heptyl, octyl, nonyl, decyl, etc. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy. The term “alkylene” refers to a divalent alkyl radical.

As used herein, the term “heteroalkyl” refers to an alkyl group as described herein, wherein one or more carbon atoms are optionally and independently replaced with heteroatom selected from N, O, and S. The term “heteroalkylene” refers to a divalent heteroalkyl radical.

As used herein, the term “carbocycle” refers to a saturated or partially unsaturated, monocyclic, fused bicyclic, or bridged polycyclic ring assembly containing from 3 to 12 ring atoms, or the number of atoms indicated. Carbocycles can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Carbocyclic groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

Unsaturated carbocyclic groups also include aryl groups. The term “aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

A “divalent” carbocycle refers to a carbocyclic group having two points of attachment for covalently linking two moieties in a molecule or material. Carbocycles can be substituted or unsubstituted. “Substituted carbocycle” groups can be substituted with one or more groups selected from halo, hydroxy, amino, alkylamino, amido, acyl, nitro, cyano, and alkoxy.

As used herein, the term “heterocycle” refers to heterocycloalkyl groups and heteroaryl groups. “Heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can be oxidized to form moieties such as, but not limited to, —S(O)— and —S(O)₂—. Heteroaryl groups can include any number of ring atoms, such as 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. “Substituted heteroaryl” groups can be substituted with one or more groups selected from halo, hydroxy, amino, oxo (═O), alkylamino, amido, acyl, nitro, cyano, and alkoxy.

Heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

“Heterocyclyl,” by itself or as part of another substituent, refers to a saturated ring system having from 3 to 12 ring members and from 1 to 4 heteroatoms of N, O and S. Additional heteroatoms can also be useful, including, but not limited to, B, Al, Si and P. The heteroatoms can be oxidized to form moieties such as, but not limited to, —S(O)— and —S(O)₂—. Heterocyclyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocyclyl groups, such as 1, 2, 3, or 4, or 1 to 2, 1 to 3, 1 to 4, 2 to 3, 2 to 4, or 3 to 4. The heterocyclyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocyclyl groups can also be fused to aromatic or non-aromatic ring systems to form members including, but not limited to, indoline. Heterocyclyl groups can be unsubstituted or substituted. “Substituted heterocyclyl” groups can be substituted with one or more groups selected from halo, hydroxy, amino, oxo (═O), alkylamino, amido, acyl, nitro, cyano, and alkoxy.

Heterocyclyl groups can be linked via any position on the ring. For example, aziridine can be 1- or 2-aziridine, azetidine can be 1- or 2- azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

As used herein, the terms “halo” and “halogen,” by themselves or as part of another substituent, refer to a fluorine, chlorine, bromine, or iodine atom.

As used herein, the term “carbonyl,” by itself or as part of another substituent, refers to —C(O)—, i.e., a carbon atom double-bonded to oxygen and bound to two other groups in the moiety having the carbonyl.

As used herein, the term “amino” refers to a moiety —NR₃, wherein each R group is H or alkyl. An amino moiety can be ionized to form the corresponding ammonium cation.

As used herein, the term “hydroxy” refers to the moiety —OH.

As used herein, the term “cyano” refers to a carbon atom triple-bonded to a nitrogen atom (i.e., the moiety —C≡N).

As used herein, the term “carboxy” refers to the moiety —C(O)OH. A carboxy moiety can be ionized to form the corresponding carboxylate anion.

As used herein, the term “amido” refers to a moiety —NRC(O)R or —C(O)NR₂, wherein each R group is H or alkyl.

As used herein, the term “nitro” refers to the moiety —NO₂.

As used herein, the term “oxo” refers to an oxygen atom that is double-bonded to a compound (i.e., O═).

As used herein, the terms “treat,” “treatment,” and “treating” refer to any indicia of success in the treatment or amelioration of an injury, pathology, condition, or symptom (e.g., cognitive impairment), including any objective or subjective parameter such as abatement; remission; diminishing of symptoms or making the symptom, injury, pathology or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter; including, e.g., the result of a physical examination.

As used herein, the term “cancer” refers to conditions including solid cancers, lymphomas, and leukemias. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, stomach (gastric) cancer, cancer of the central nervous system, skin cancer (e.g., melanoma), choriocarcinoma, head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia, and multiple myeloma.

As used herein the terms “effective amount” and “therapeutically effective amount” refer to a dose of a substance such as an immunoconjugate that produces therapeutic effects for which it is administered. The exact dose will depend on the purpose of the treatment, and will be ascertainable by one skilled in the art using known techniques (see, e.g., Lieberman, Pharmaceutical Dosage Forms (volumes 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition, 2006, Brunton, ed., McGraw-Hill; and Remington: The Science and Practice of Pharmacy, 21st Edition, 2005, Hendrickson, Ed., Lippincott, Williams & Wilkins).

As used herein, the term “subject” refers to animals such as mammals, including, but not limited to, primates (e.g., humans), cows, sheep, goats, horses, dogs, cats, rabbits, rats, mice and the like. In certain embodiments, the subject is a human.

As used herein, the term “administering” refers to parenteral, intravenous, intraperitoneal, intramuscular, intratumoral, intralesional, intranasal or subcutaneous administration, oral administration, administration as a suppository, topical contact, intrathecal administration, or the implantation of a slow-release device, e.g., a mini-osmotic pump, to the subject.

As used herein, the structure

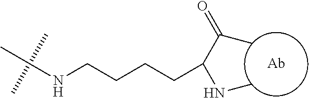

is an antibody with residue

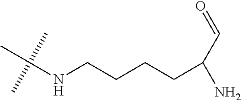

representing a lysine residue of the antibody, wherein “ [symbol] ” represents a point of attachment to a linker. Accordingly, Ab is the remainder of an antibody containing the depicted at least one lysine residue. The structure “ [symbol] ”, which represents the point of attachment to the linker, can represent the point of attachment to Z, Z¹, or G₂, which are described herein and present in immunoconjugates of Formula II, Formula III, and Formula IV, respectively.

The terms “about” and “around,” as used herein to modify a numerical value, indicate a close range surrounding that explicit value. If “X” were the value, “about X” or “around X” would indicate a value from 0.9X to 1.1X, e.g., from 0.95X to 1.05X or from 0.99X to 1.01X. Any reference to “about X” or “around X” specifically indicates at least the values X, 0.95X, 0.96X, 0.97X, 0.98X, 0.99X, 1.01X, 1.02X, 1.03X, 1.04X, and 1.05X. Thus, “about X” and “around X” are intended to teach and provide written description support for a claim limitation of, e.g., “0.98X.”

### Antibody Adjuvant Conjugates

The invention provides immunoconjugates containing an antibody construct comprising an antigen binding domain and an Fc domain, an adjuvant moiety, and a linker, wherein each adjuvant moiety is covalently bonded to the antibody via the linker.

Immunoconjugates as described herein can provide an unexpectedly increased activation response of an antigen presenting cell (APC). This increased activation can be detected in vitro or in vivo. In some cases, increased APC activation can be detected in the form of a reduced time to achieve a specified level of APC activation. For example, in an in vitro assay, % APC activation can be achieved at an equivalent dose with an immunoconjugate within 1%, 10%, or 50% of the time required to receive the same or similar percentage of APC activation with a mixture of unconjugated antibody and TLR agonist, under otherwise identical concentrations and conditions. In some cases, an immunoconjugate can activate APCs (e.g., dendritic cells) and/or NK cells in a reduced amount of time. For example, in some cases, an antibody TLR agonist mixture can activate APCs (e.g., dendritic cells) and/or NK cells and/or induce dendritic cell differentiation after incubation with the mixture for 2, 3, 4, 5, 1-5, 2-5, 3-5, or 4-7 days; while, in contrast immunoconjugates described herein can activate and/or induce differentiation within 4 hours, 8 hours, 12 hours, 16 hours, or 1 day, under otherwise identical concentrations and conditions. Alternatively, the increased APC activation can be detected in the form of a reduced concentration of immunoconjugate required to achieve an amount (e.g., percent APCs), level (e.g., as measured by a level of upregulation of a suitable marker), or rate (e.g., as detected by a time of incubation required to activate) of APC activation.

Immunoconjugates of the invention must include an Fc region. Non-FcR binding proteins do not activate myeloid cells when conjugated to adjuvants of the invention.

In one embodiment, the immunoconjugates of the invention provide more than a 5% increase in activity compared to the immunoconjugates of the prior art (for example, the immunoconjugates disclosed in U.S. Pat. No. 8,951,528). In another embodiment, the immunoconjugates of the invention provide more than a 10%, 15%, 20%, 25%, 30%, 35%, 40%, 45%, 50%, 55%, 60%, 65%, or 70% increase in activity compared to the immunoconjugates of the prior art. The increase in activity can be assessed by any suitable means, many of which are known to those ordinarily skilled in the art and can include myeloid activation or assessment by cytokine secretion.

In one embodiment, the immunoconjugates of the invention provide an improved drug to adjuvant ratio. In some embodiments, the average number of adjuvant moieties per immunoconjugate ranges from about 1 to about 10. The desirable drug to adjuvant ratio can be determined by an ordinarily skilled artisan depending on the desired effect of the treatment. For example, a drug to adjuvant ratio of greater than 1.2 may be desired. In an embodiment, a drug to adjuvant ratio of greater than 0.2, 0.4, 0.6, 0.8, 1, 1.2, 1.4, 1.6. 1.8, 2.0, 2.2, 2.4, 2.6, 2.8, 3.0, 3.2, 3.4, 3.6, 3.8, 4.0, 5.0, 6.0, 7.0, 8.0, or 9.0 may be desired. In another embodiment, a drug to adjuvant ratio of less than 10.0, 9.0, 8.0, 7.0, 6.0, 5.0, 4.0, 3.8, 3.6, 3.4, 3.2, 3.0, 2.8, 2.6, 2.4, 2.2, 2.0, 1.8, 1.6, 1.4, 1.2, 0.8, 0.6, 0.4 or 0.2 may be desirable. The drug to adjuvant ratio can be assessed by any suitable means, many of which are known to those ordinarily skilled in the art.

In some embodiments, the immunoconjugate has a structure according to Formula II:

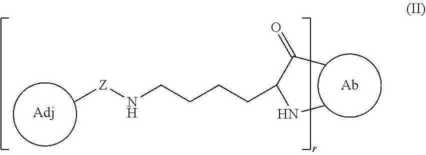

wherein

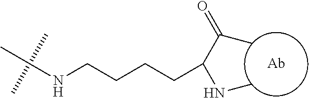

is an antibody with residue

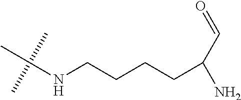

representing a lysine residue of the antibody, wherein “ [symbol] ” represents a point of attachment to Z; Adj is an adjuvant; subscript r is an integer from 1 to 10; and Z is a divalent linking moiety having an ethylene glycol group or a glycine residue. Z preferably is bonded to the adjuvant via an amide bond, a C—N single bond, a C—O single bond, or a C—C single bond, and to the antibody via an amide bond or a C—N single bond. In some embodiments, Z is bonded to a nitrogen group of the adjuvant and a nitrogen group of the antibody. As used herein, the term “nitrogen group” refers to an unsubstituted or substituted amine atom present in the adjuvant or antibody. In such embodiments, Z is bonded to adjacent nitrogen groups via amide bonds, C—N single bonds, or a combination thereof.

### Adjuvants

In some embodiments, the adjuvant moiety is a compound that elicits an immune response. In some embodiments, the adjuvant moiety is a pattern recognition receptor (“PRR”) agonist. Any adjuvant capable of activating a pattern recognition receptor (PRR) can be installed in the immunoconjugates of the invention. As used herein, the terms “Pattern recognition receptor” and “PRR” refer to any member of a class of conserved mammalian proteins which recognize pathogen-associated molecular patterns (“PAMPs”) or damage-associated molecular patterns (“DAMPs”), and act as key signaling elements in innate immunity. Pattern recognition receptors are divided into membrane-bound PRRs, cytoplasmic PRRs, and secreted PRRs. Examples of membrane-bound PRRs include Toll-like receptors (“TLRs”) and C-type lectin receptors (“CLRs”). Examples of cytoplasmic PRRs include NOD-like receptors (“NLRs”) and Rig-I-like receptors (“RLRs”). In some embodiments, the immunoconjugate can have more than one distinct PRR adjuvant moiety.

In certain embodiments, the adjuvant moiety in an immunoconjugate of the invention is a Toll-like receptor (TLR) agonist. Suitable TLR agonists include TLR1, TLR2, TLR3, TLR4, TLR5, TLR6, TLR7, TLR8, TLR9, TLR10, TLR11, or any combination thereof (e.g., TLR7/8 agonists). Any adjuvant capable of activating a Toll-like receptor (TLR) can be installed in the immunoconjugates of the invention. Toll-like receptors (TLRs) are type-I transmembrane proteins that are responsible for initiation of innate immune responses in vertebrates. TLRs recognize a variety of pathogen-associated molecular patterns from bacteria, viruses, and fungi and act as a first line of defense against invading pathogens. TLRs elicit overlapping yet distinct biological responses due to differences in cellular expression and in the signaling pathways that they initiate. Once engaged (e.g., by a natural stimulus or a synthetic TLR agonist) TLRs initiate a signal transduction cascade leading to activation of NF-κB via the adapter protein myeloid differentiation primary response gene 88 (MyD88) and recruitment of the IL-1 receptor associated kinase (IRAK). Phosphorylation of IRAK then leads to recruitment of TNF-receptor associated factor 6 (TRAF6), which results in the phosphorylation of the NF-κB inhibitor I-κB. As a result, NF-κB enters the cell nucleus and initiates transcription of genes whose promoters contain NF-κB binding sites, such as cytokines. Additional modes of regulation for TLR signaling include TIR-domain containing adapter-inducing interferon-β (TRIF)-dependent induction of TRAF6 and activation of MyD88 independent pathways via TRIF and TRAF3, leading to the phosphorylation of interferon response factor three (IRF3). Similarly, the MyD88 dependent pathway also activates several IRF family members, including IRF5 and IRF7 whereas the TRIF dependent pathway also activates the NF-κB pathway.

Examples of TLR3 agonists include Polyinosine-polycytidylic acid (poly (I:C)), Polyadenylic-polyuridylic acid (poly (A:U), and poly(I)-poly(C12U).

Examples of TLR4 agonists include Lipopolysaccharide (LPS) and Monophosphoryl lipid A (MPLA).

An example of a TLR5 agonist includes Flagellin.

Examples of TLR9 agonists include single strand CpG oligodeoxynucleotides (CpG ODN). Three major classes of stimulatory CpG ODNs have been identified based on structural characteristics and activity on human peripheral blood mononuclear cells (PBMCs), in particular B cells and plasmacytoid dendritic cells (pDCs). These three classes are Class A (Type D), Class B (Type K) and Class C.

Examples of Nod Like Receptor (NLR) agonists include acylated derivative of iE-DAP, D-gamma-Glu-mDAP, L-Ala-gamma-D-Glu-mDAP, Muramyldipeptide with a C18 fatty acid chain, Muramyldipeptide, muramyl tripeptide, and N-glycosylated muramyldipeptide. p Examples of RIG-I-Like receptor (RLR) agonists include 5′ppp-dsrna (5′-pppGCAUGCGACCUCUGUUUGA-3′ [SEQ ID NO: 1]: 3′-CGUACGCUGGAGACAAACU-5′ [SEQ ID NO: 2]), and Poly(deoxyadenylic-deoxythymidylic) acid (Poly(dA:dT))

Additional immune-stimulatory compounds, such as cytosolic DNA and unique bacterial nucleic acids called cyclic dinucleotides, can be recognized by stimulator of interferon genes (“STING”), which can act a cytosolic DNA sensor. ADU-S1OO can be a STING agonist. Non-limiting examples of STING agonists include: Cyclic [G(2′,5′)pA(2′,5′)p] (2′2′-cGAMP), cyclic [G(2′,5′)pA(3′,5′)p] (2′3′-cGAMP), cyclic [G(3′,5′)pA(3′,5′)p] (3′3′-cGAMP), Cyclic di-adenylate monophosphate (c-di-AMP), 2′,5′-3′,5′-c-diAMP (2′3′-c-di-AMP), Cyclic di-guanylate monophosphate (c-di-GMP), 2′,5′-3′,5′-c-diGMP (2′3′-c-di-GMP), Cyclic di-inosine monophosphate (c-di-IMP), Cyclic di-uridine monophosphate (c-di-UMP), KIN700, KIN1148, KIN600, KIN500, KIN100, KIN101, KIN400, KIN2000, or SB-9200 can be recognized.

Any adjuvant capable of activating TLR7 and/or TLR8 can be installed in the immunoconjugates of the invention. Examples of TLR7 agonists and TLR8 agonists are described, e.g., by Vacchelli et al. (Oncolmmunology, 2: 8, e25238, DOI: 10.4161/onci.25238 (2013)) and Carson et al. (U.S. Patent Application Publication 2013/0165455, which is hereby incorporated by reference in its entirety). TLR7 and TLR8 are both expressed in monocytes and dendritic cells. In humans, TLR7 is also expressed in plasmacytoid dendritic cells (pDCs) and B cells. TLR8 is expressed mostly in cells of myeloid origin, i.e., monocytes, granulocytes, and myeloid dendritic cells. TLR7 and TLR8 are capable of detecting the presence of “foreign” single-stranded RNA within a cell, as a means to respond to viral invasion. Treatment of TLR8-expressing cells, with TLR8 agonists can result in production of high levels of IL-12, IFN-γ, IL-1, TNF-α, IL-6, and other inflammatory cytokines. Similarly, stimulation of TLR7-expressing cells, such as pDCs, with TLR7 agonists can result in production of high levels of IFN-α and other inflammatory cytokines. TLR7/TLR8 engagement and resulting cytokine production can activate dendritic cells and other antigen-presenting cells, driving diverse innate and acquired immune response mechanisms leading to tumor destruction.

Examples of TLR7, TLR8 or TLR7/8 agonists include but are not limited to: Gardiquimod (1-(4-amino-2-ethylaminomethylimidazo[4,5-c]quinolin-1-yl)-2-methylpropan-2-ol), Imiquimod (R837) (agonist for TLR7), loxoribine (agonist for TLR7), IRM1 (1-(2-amino-2-methylpropyl)-2-(ethoxymethyl)-1H-imidazo-[4,5-c]quinolin-4-amine), IRM2 (2-methyl-1-[2-(3-pyridin-3-ylpropoxy)ethyl]-1H-imidazo[4,5-c]quinolin-4-annne) (agonist for TLR8), IRM3 (N-(2-[2-[4-amino-2-(2-methoxyethyl)-1H-imidazo[4,5-c]quinolin-1-yl]ethoxy]ethyl)-N-methylcyclohexanecarboxamide) (agonist for TLR8), CL097 (2-(ethoxymethyl)-1H-imidazo[4,5-c]quinolin-4-amine) (agonist for TLR7/8), CL307 (agonist for TLR7), CL264 (agonist for TLR7), Resiquimod (agonist for TLR7/8), 3M-052/MEDI9197 (agonist for TLR7/8), SD-101 (N-[(4S)-2,5-dioxo-4-imidazolidinyl]-urea) (agonist for TLR7/8), motolimod (2-amino-N,N-dipropyl-8-[4-(pyrrolidine-1-carbonyl)phenyl]-3H-1-benzazepine-4-carboxamide) (agonist for TLR8), CL075 (3M002, 2-propylthiazolo[4,5-c]quinolin-4-amine) (agonist for TLR7/8), and TL8-506 (3H-1-benzazepine-4-carboxylic acid, 2-amino-8-(3-cyanophenyl)-, ethyl ester) (agonist for TLR8).

Examples of TLR2 agonists include but are not limited to an agent comprising N-α-palmitoyl-S-[2,3-bis(palmitoyloxy)-(2RS)-propyl]-L-cysteine, palmitoyl-Cys((RS)-2,3-di(palmitoyloxy)-propyl) (“Pam3Cys”), e.g., Pam3Cys, Pam3Cys-Ser-(Lys)4 (also known as “Pam3Cys-SKKKK” and “Pam₃CSK₄”), Triacyl lipid A (“OM-174”), Lipoteichoic acid (“LTA”), peptidoglycan, and CL419 (S-(2,3-bis(palmitoyloxy)-(2RS)propyl)-(R)-cysteinyl spermine).

An example of a TLR2/6 agonist is Pam2CSK₄ (S-[2,3-bis(palmitoyloxy)-(2RS)-propyl]-[R]-cysteinyl-[S]-seryl-[S]-lysyl-[S]-lysyl-[S]-lysyl-[S]-lysine×3 CF3COOH).

Examples of TLR2/7 agonist include CL572 (S-(2-myristoyloxy ethyl)-(R)-cysteinyl 4-((6-amino-2-(butylamino)-8-hydroxy-9H-purin-9-yl)methyl) aniline), CL413 (S-(2,3-bis(palmitoyloxy)-(2RS)propyl)-(R)-cysteinyl-(S)-seryl-(S)-lysyl-(S)-lysyl-(S)-lysyl-(S)-lysyl 4-((6-amino-2-(butylamino)-8-hydroxy-9H-purin-9-yl)methyl)aniline), and CL401 (S-(2,3-bis(palmitoyloxy)-(2RS)propyl)-(R)-cysteinyl 4-((6-amino-2(butyl amino)-8-hydroxy-9H-purin-9-yl)methyl) aniline).

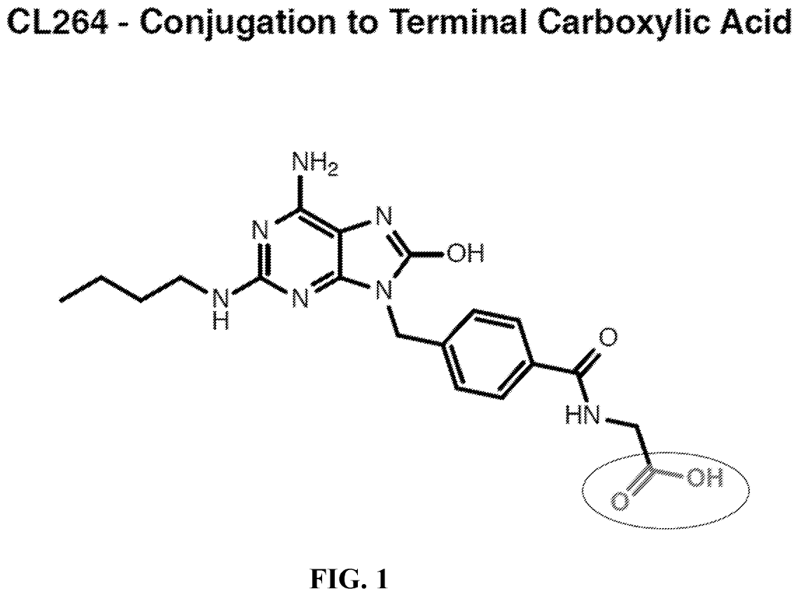

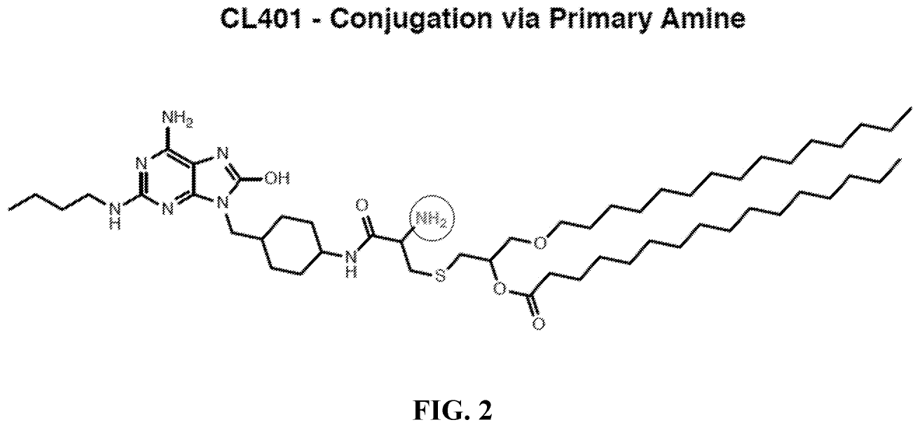

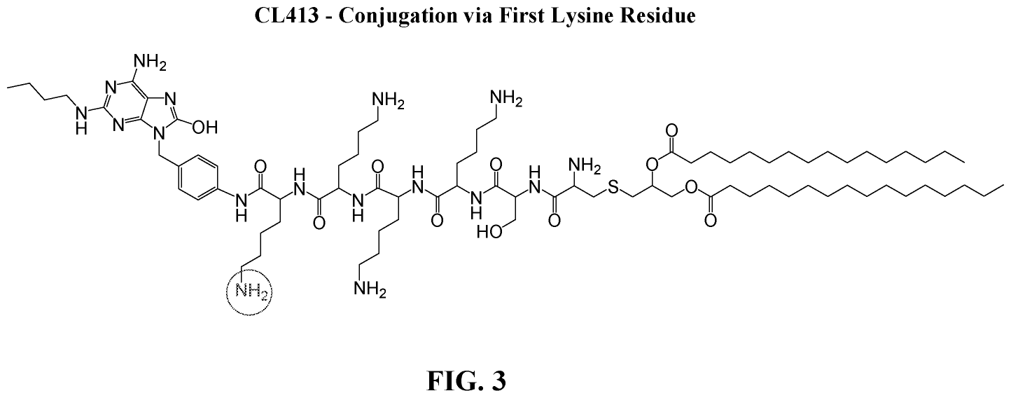

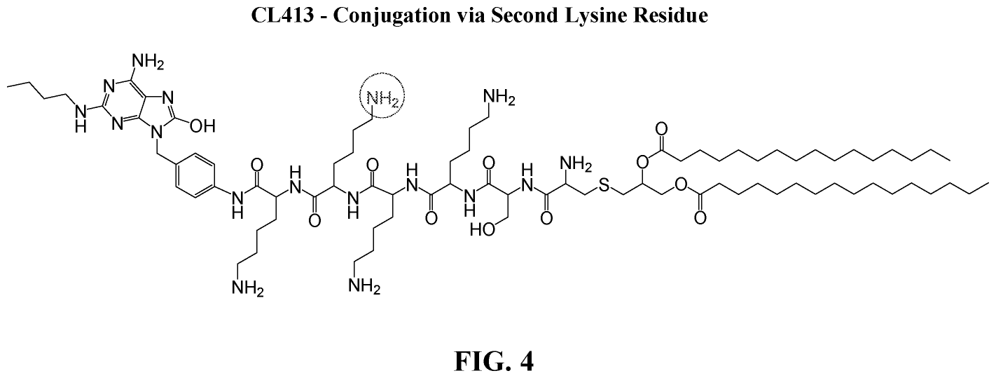

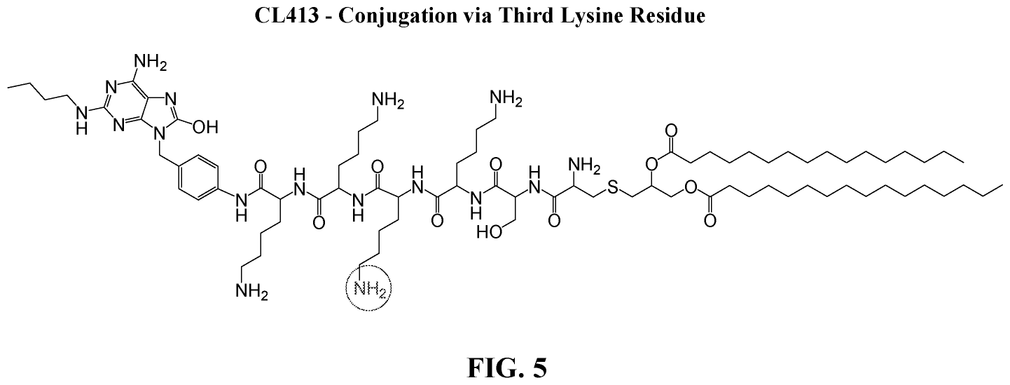

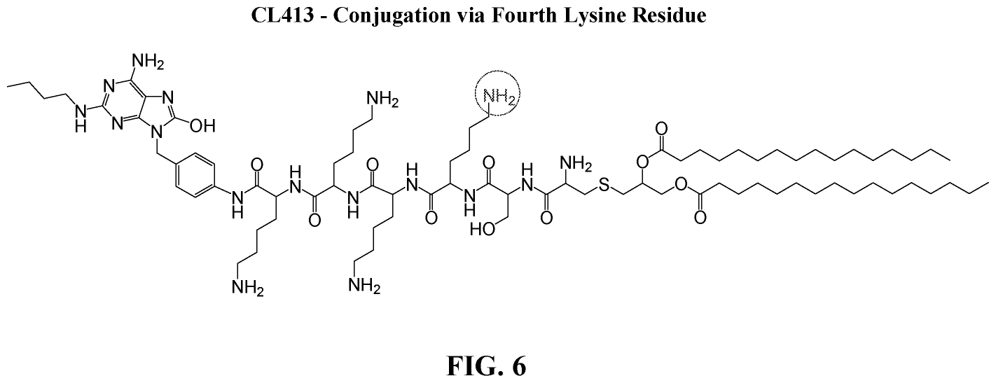

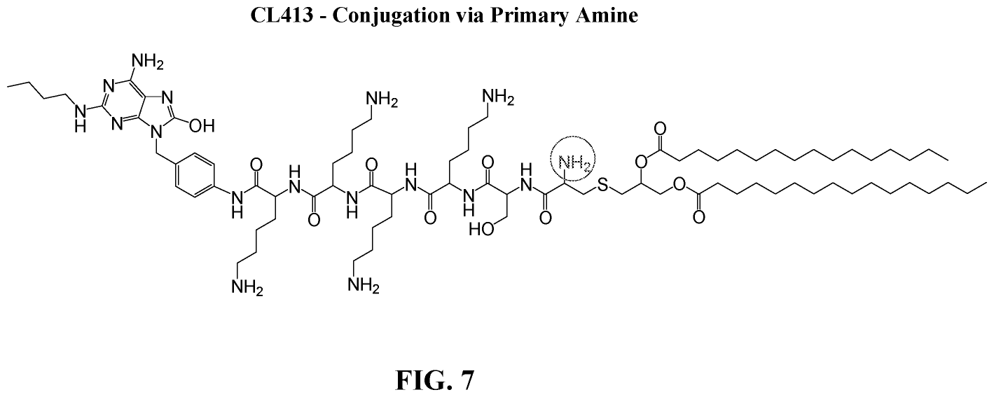

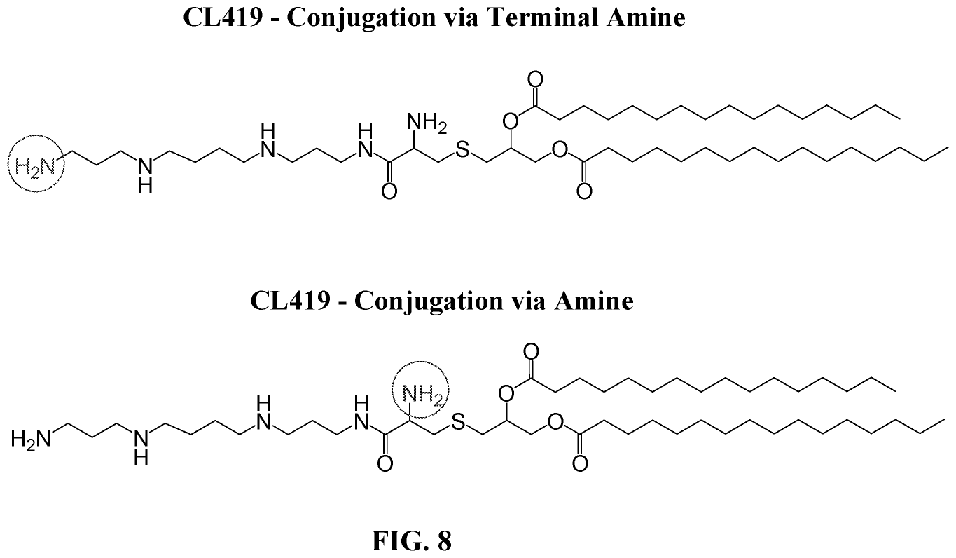

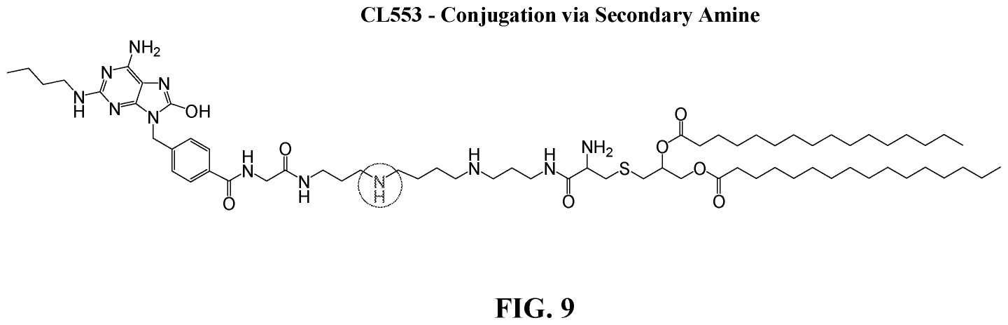

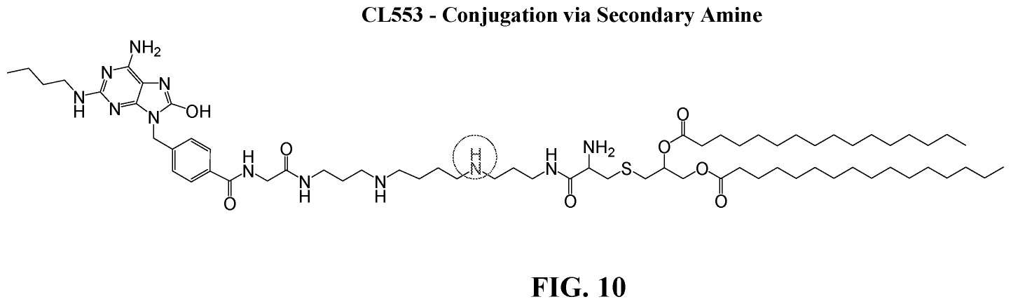

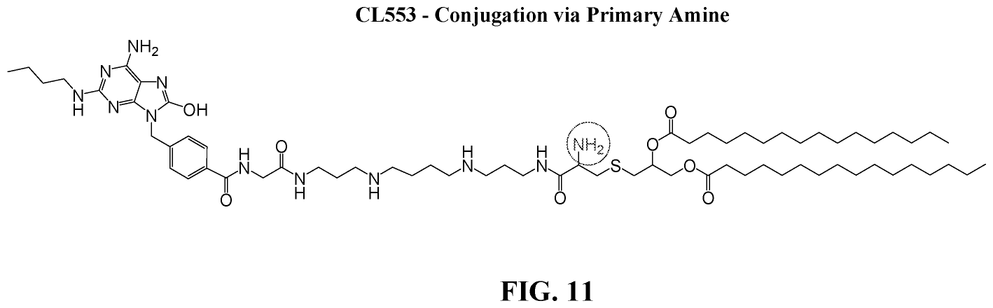

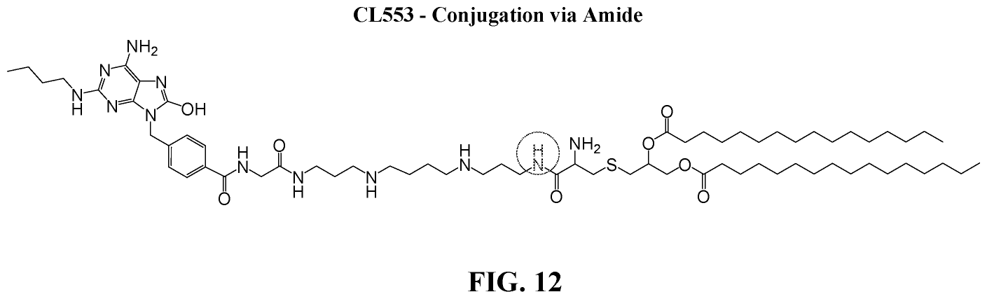

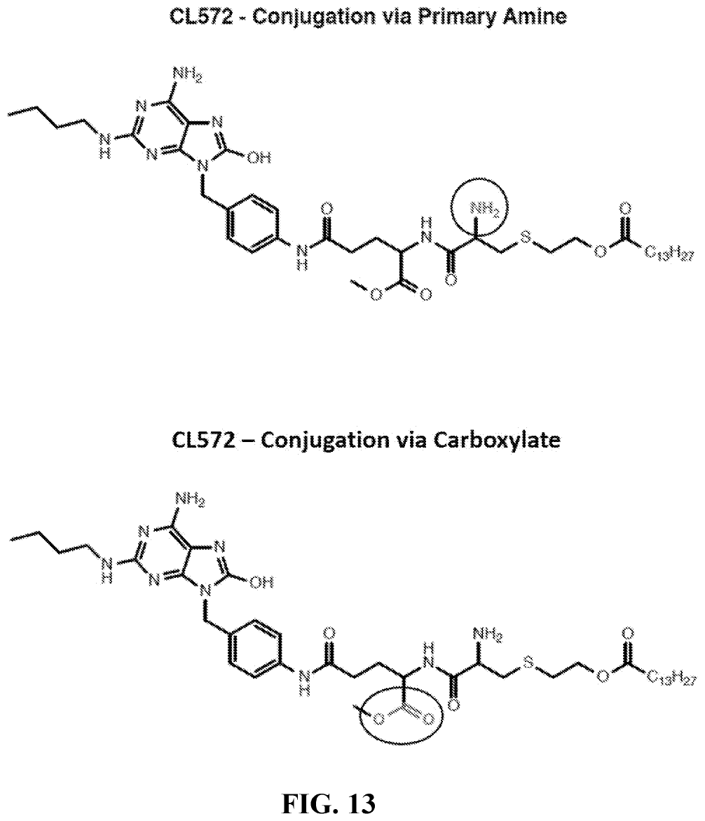

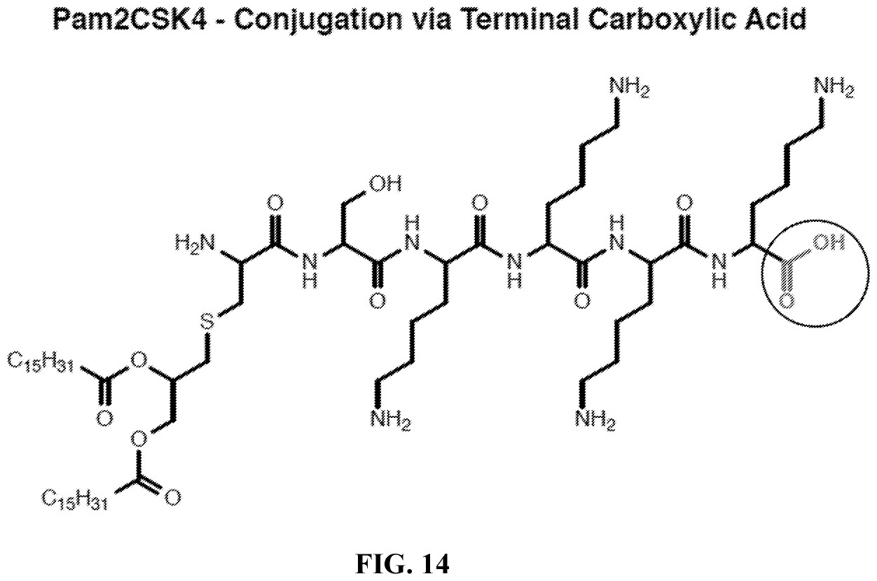

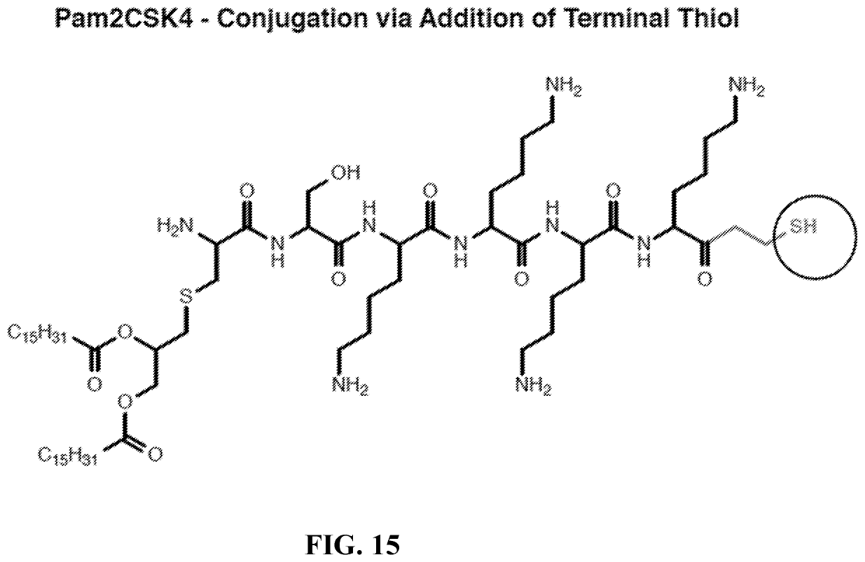

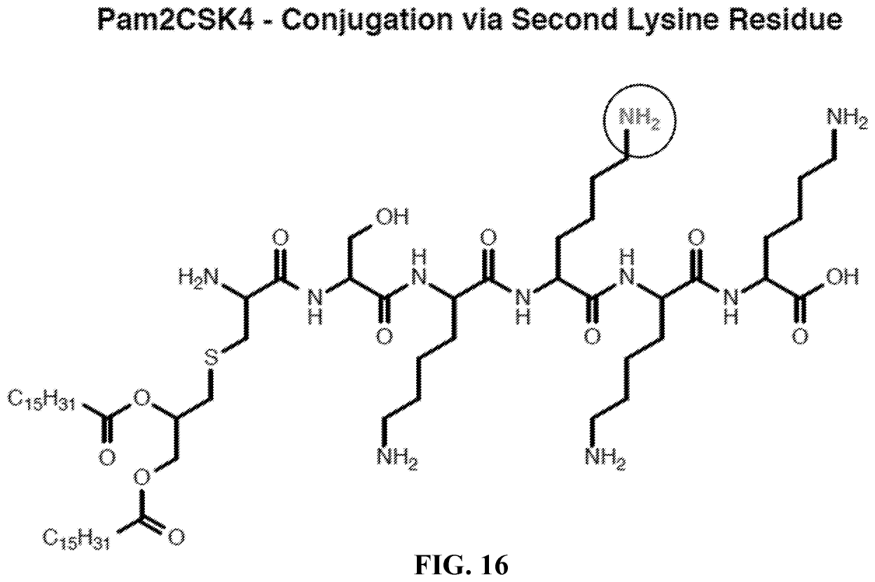

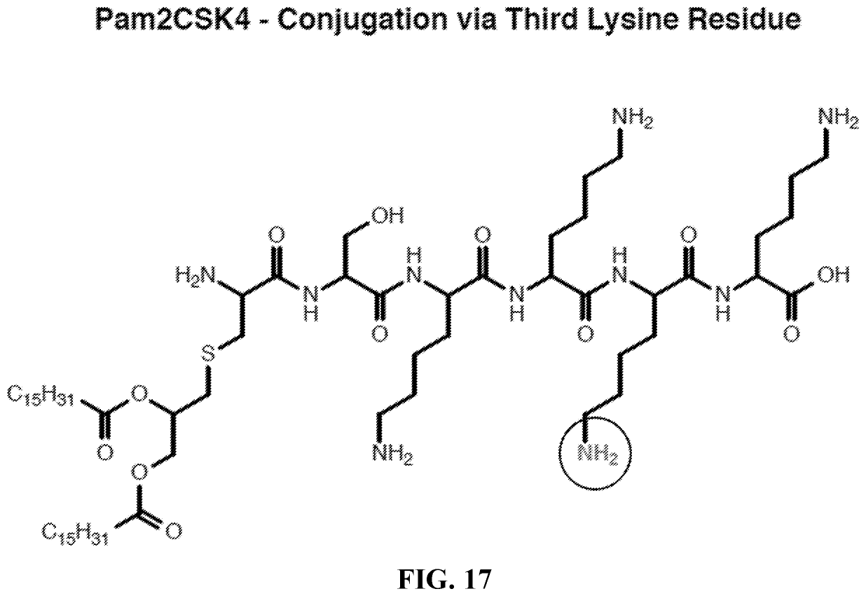

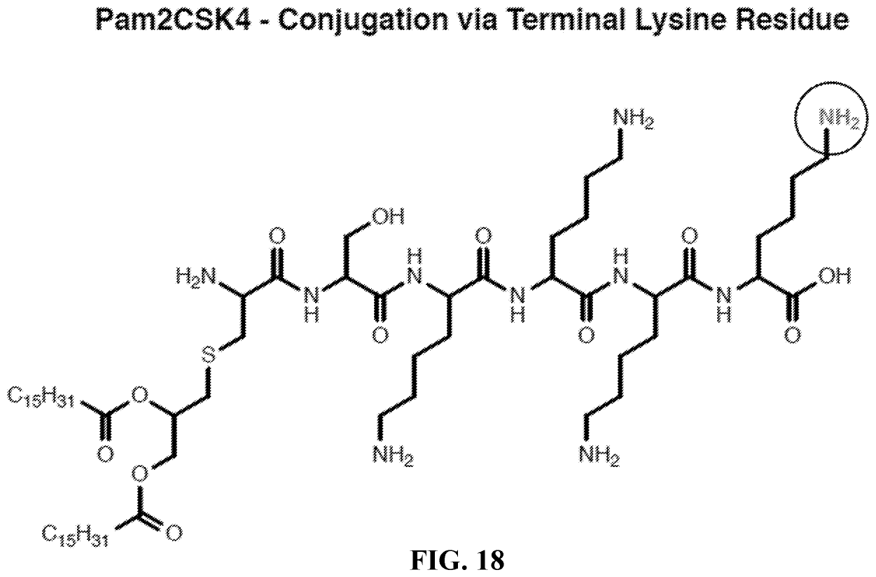

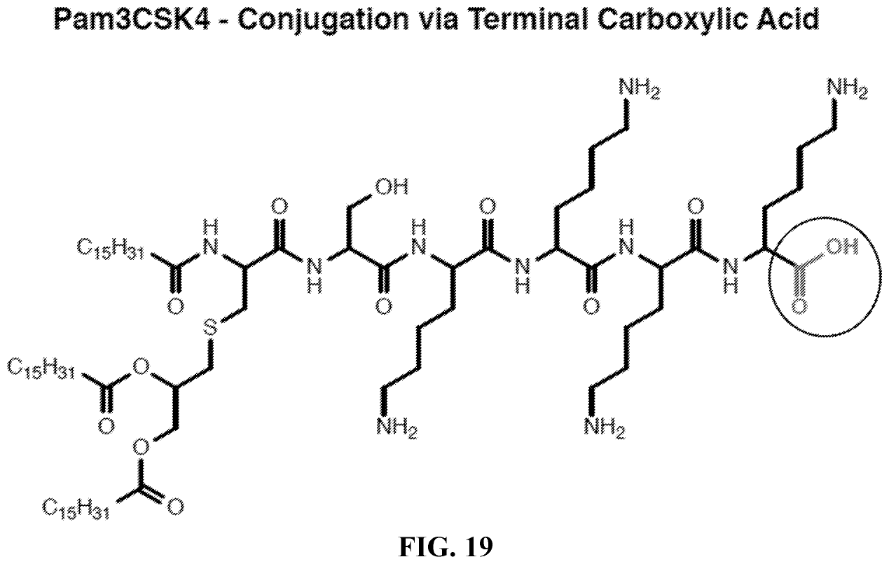

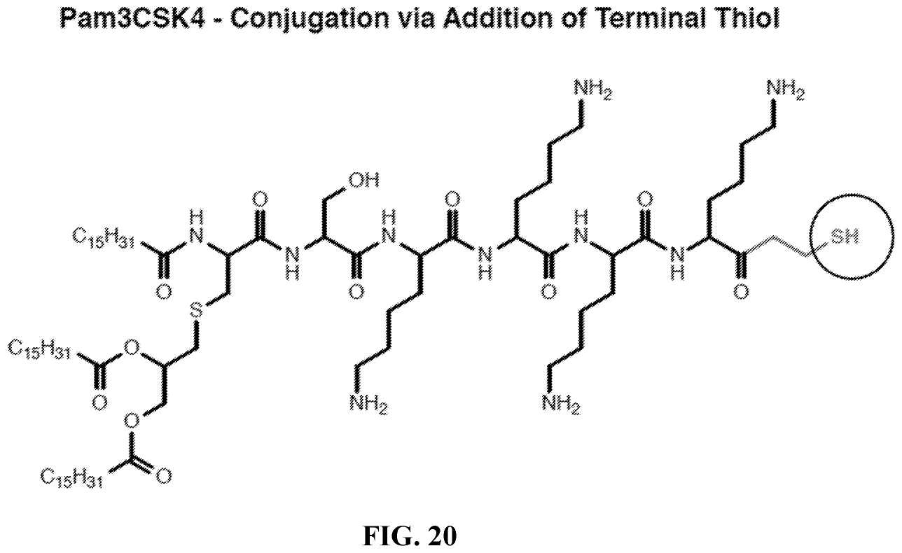

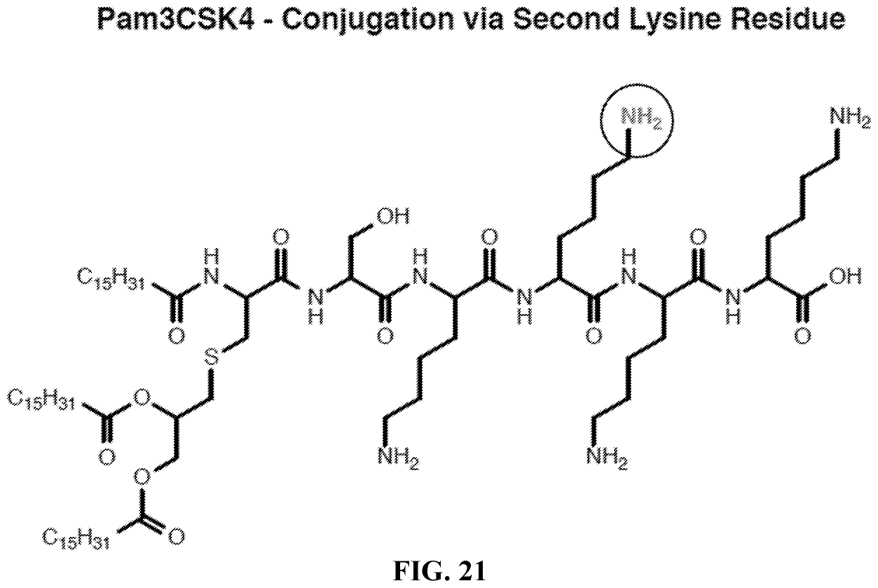

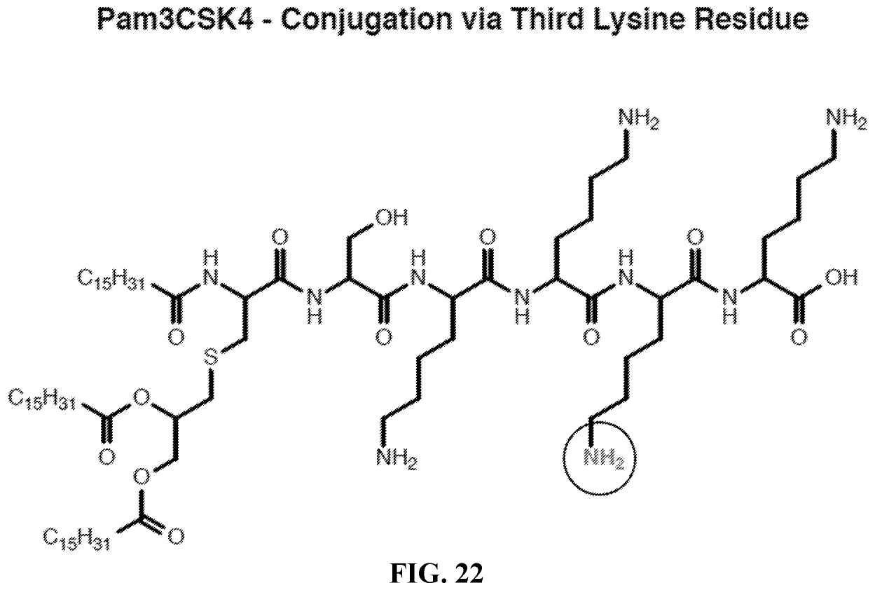

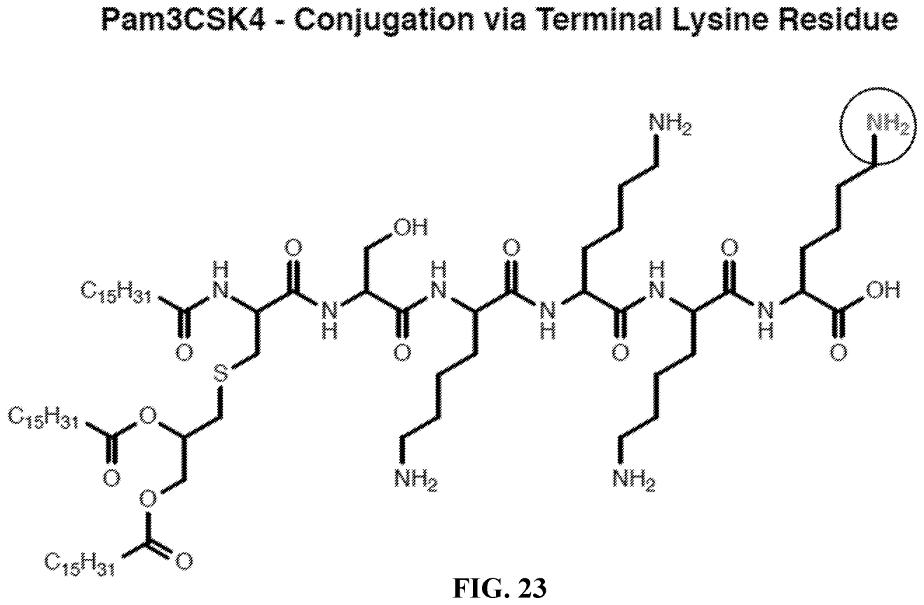

FIGS. 1-23 show where TLR agonists CL264, CL401, CL413, CL419, CL553, CL572, Pam₃CSK₄, and Pam₂CSK₄ could be linked to immunoconjugates of the invention while maintaining their adjuvant activity. Specifically, the location where the linker should be attached to the adjuvant is circled.

In some embodiments, the adjuvant moiety is an imidazoquinoline compound. Examples of useful imidazoquinoline compounds include those described in U.S. Pat. Nos. 5,389,640; 6,069,149; and 7,968,562, which are hereby incorporated by reference in their entirety.

In some embodiments, the adjuvant (“Adj”) is of formula:

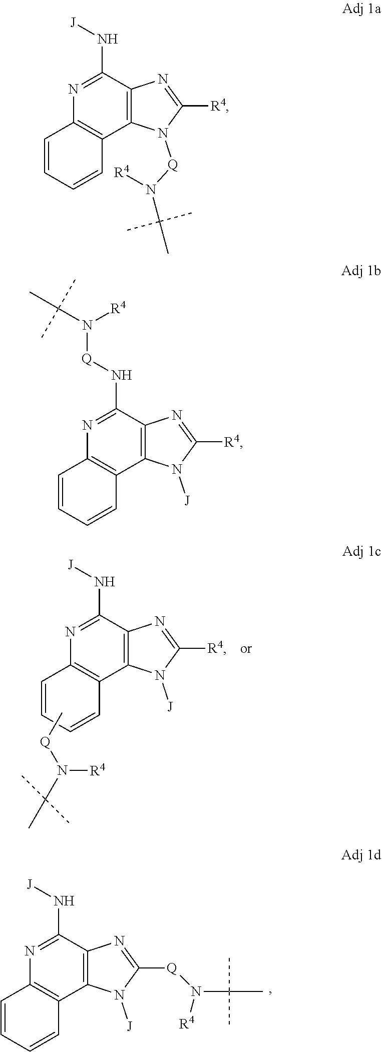

wherein each J independently is hydrogen, OR⁴, or R⁴; each R⁴ independently is hydrogen, or an alkyl, heteroalkyl, cycloalkyl, heterocycloalkyl, aryl, heteroaryl, arylalkyl, or heteroarylalkyl group comprising from 1 to 8 (i.e., 1, 2, 3, 4, 5, 6, 7, or 8) carbon units; Q is optionally present and is an alkyl, heteroalkyl, cycloalkyl, heterocycloalkyl, aryl, heteroaryl, arylalkyl, or heteroarylalkyl group comprising from 1 to 8 (i.e., 1, 2, 3, 4, 5, 6, 7, or 8) carbon units; and the dashed line (“ [symbol] ”) represents the point of attachment of the adjuvant.

In certain embodiments, Q is present. In certain embodiments, the adjuvant (“Adj”) is of formula:

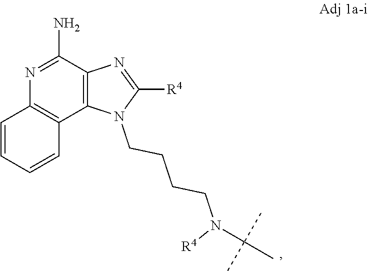

wherein each R⁴ independently is selected from the group consisting of hydrogen, or alkyl, heteroalkyl, cycloalkyl, heterocycloalkyl, aryl, heteroaryl, arylalkyl, and heteroarylalkyl group comprising from 1 to 8 (i.e., 1, 2, 3, 4, 5, 6, 7, or 8) carbon units and the dashed line (“ [symbol] ”) represents the point of attachment of the adjuvant.

In some embodiments, the adjuvant (“Adj”) is of formula:

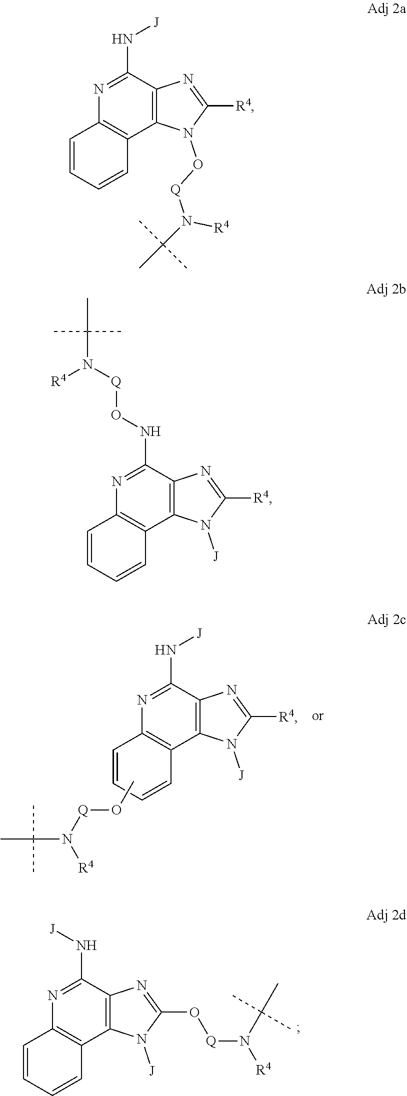

wherein J is hydrogen, OR⁴, or R⁴; each R⁴ independently is hydrogen, or alkyl, heteroalkyl, cycloalkyl, heterocycloalkyl, aryl, heteroaryl, arylalkyl, and heteroarylalkyl group comprising from 1 to 8 (i.e., 1, 2, 3, 4, 5, 6, 7, or 8) carbon units; Q is selected from the group consisting of alkyl, or heteroalkyl, cycloalkyl, heterocycloalkyl, aryl, heteroaryl, arylalkyl, and heteroarylalkyl group comprising from 1 to 8 (i.e., 1, 2, 3, 4, 5, 6, 7, or 8) carbon units; and the dashed line (“ [symbol] ”) represents the point of attachment of the adjuvant.

In certain embodiments, the adjuvant (“Adj”) is of formula:

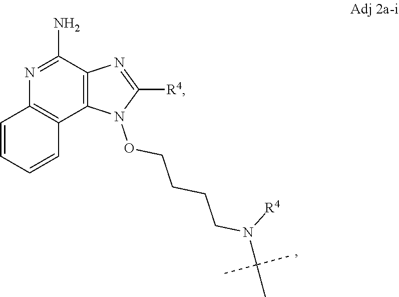

wherein each R⁴ independently is selected from the group consisting of hydrogen, or alkyl, heteroalkyl, cycloalkyl, heterocycloalkyl, aryl, heteroaryl, arylalkyl, and heteroarylalkyl group comprising from 1 to 8 (i.e., 1, 2, 3, 4, 5, 6, 7, or 8) carbon units and the dashed line (“ [symbol] ”) represents the point of attachment of the adjuvant.

In some embodiments, the adjuvant (“Adj”) is of formula:

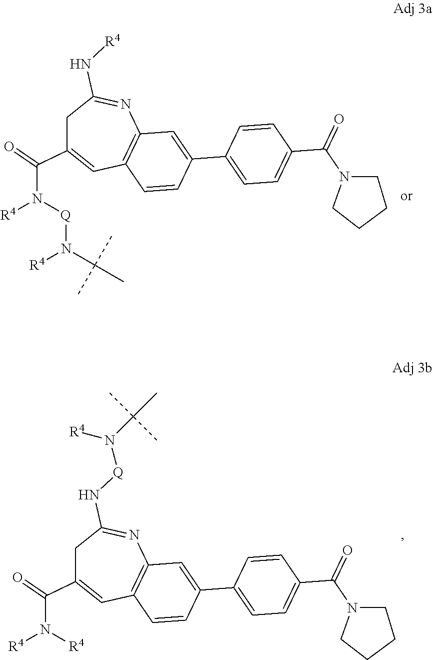

wherein each R⁴ independently is hydrogen, or alkyl, heteroalkyl, cycloalkyl, heterocycloalkyl, aryl, heteroaryl, arylalkyl, or heteroarylalkyl group comprising from 1 to 8 (i.e., 1, 2, 3, 4, 5, 6, 7, or 8) carbon units; Q is alkyl, heteroalkyl, cycloalkyl, heterocycloalkyl, aryl, heteroaryl, arylalkyl, or heteroarylalkyl group comprising from 1 to 8 (i.e., 1, 2, 3, 4, 5, 6, 7, or 8) carbon units; and the dashed line (“ [symbol] ”) represents the point of attachment of the adjuvant.

In some embodiments, the adjuvant (“Adj”) is of formula:

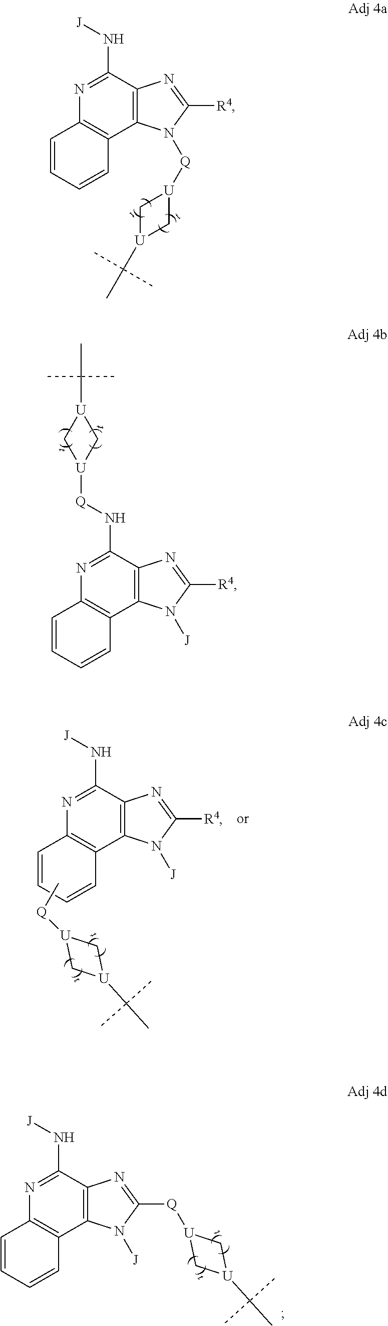

wherein each J independently is hydrogen, OR⁴, or R⁴; each R⁴ independently is hydrogen, or an alkyl, heteroalkyl, cycloalkyl, heterocycloalkyl, aryl, heteroaryl, arylalkyl, or heteroarylalkyl group comprising from 1 to 8 (i.e., 1, 2, 3, 4, 5, 6, 7, or 8) carbon units; each U independently is CH or N wherein at least one U is N; each subscript t independently is an integer from 1 to 3 (i.e., 1, 2, or 3); Q is optionally present and is an alkyl, heteroalkyl, cycloalkyl, heterocycloalkyl, aryl, heteroaryl, arylalkyl, or heteroarylalkyl group comprising from 1 to 8 (i.e., 1, 2, 3, 4, 5, 6, 7, or 8) carbon units; and the dashed line (“ [symbol] ”) represents the point of attachment of the adjuvant.

In certain embodiments, Q is present. In certain embodiments, the adjuvant (“Adj”) is of formula:

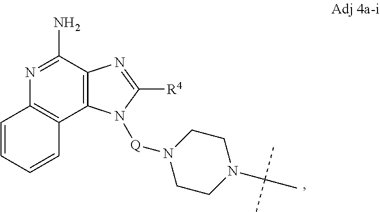

wherein R⁴ is selected from the group consisting of hydrogen, or alkyl, heteroalkyl, cycloalkyl, heterocycloalkyl, aryl, heteroaryl, arylalkyl, and heteroarylalkyl group comprising from 1 to 8 (i.e., 1, 2, 3, 4, 5, 6, 7, or 8) carbon units Q is an alkyl, heteroalkyl, cycloalkyl, heterocycloalkyl, aryl, heteroaryl, arylalkyl, or heteroarylalkyl group comprising from 1 to 8 (i.e., 1, 2, 3, 4, 5, 6, 7, or 8) carbon units; and the dashed line (“ [symbol] ”) represents the point of attachment of the adjuvant.

In some embodiments, the adjuvant (“Adj”) is of formula:

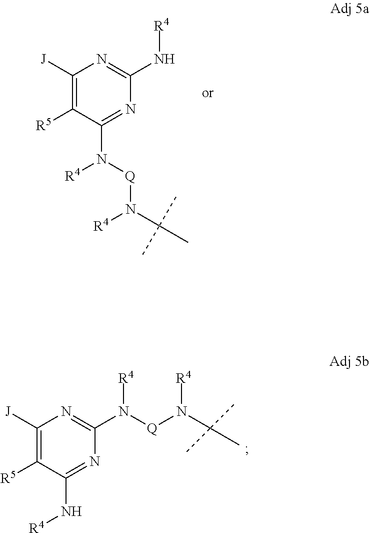

wherein J is hydrogen, OR⁴, or R⁴; each R⁴ independently is hydrogen, or an alkyl, heteroalkyl, cycloalkyl, heterocycloalkyl, aryl, heteroaryl, arylalkyl, or heteroarylalkyl group comprising from 1 to 8 (i.e., 1, 2, 3, 4, 5, 6, 7, or 8) carbon units; R⁵ is hydrogen, or an alkyl, heteroalkyl, cycloalkyl, heterocycloalkyl, aryl, heteroaryl, arylalkyl, or heteroarylalkyl group comprising from 1 to 10 (i.e., 1, 2, 3, 4, 5, 6, 7, 8, 9, or 10) carbon units; Q is an alkyl, heteroalkyl, cycloalkyl, heterocycloalkyl, aryl, heteroaryl, arylalkyl, or heteroarylalkyl group comprising from 1 to 8 (i.e., 1, 2, 3, 4, 5, 6, 7, or 8) carbon units; and the dashed line (“ [symbol] ”) represents the point of attachment of the adjuvant.

In certain embodiments, the adjuvant (“Adj”) is of formula:

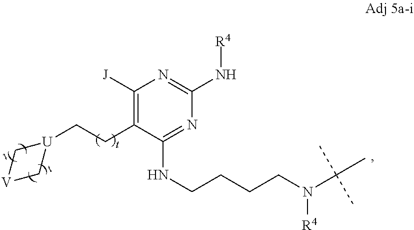

wherein J is hydrogen, OR⁴, or R⁴; each R⁴ independently is selected from the group consisting of hydrogen, or alkyl, heteroalkyl, cycloalkyl, heterocycloalkyl, aryl, heteroaryl, arylalkyl, and heteroarylalkyl group comprising from 1 to 8 (i.e., 1, 2, 3, 4, 5, 6, 7, or 8) carbon units; U is CH or N; V is CH₂, O, or NH; each subscript t independently is an integer from 1 to 3 (i.e., 1, 2, or 3); and the dashed line (“ [symbol] ”) represents the point of attachment of the adjuvant.

In some embodiments, the adjuvant (“Adj”) is of formula:

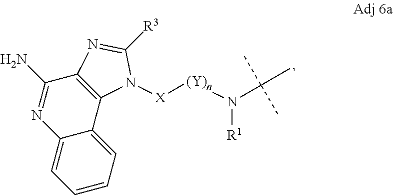

wherein R¹ is selected from H and C₁₋₄ alkyl; R³ is selected from C₁₋₆ alkyl and 2-to 6-membered heteroalkyl, each of which is optionally substituted with one or more members selected from the group consisting of halo, hydroxy, amino, oxo (═O), alkylamino, amido, acyl, nitro, cyano, and alkoxy; X is selected from O and CH₂; each Y is independently CHR², wherein R² is selected from H, OH, and NH₂, subscript n is an integer from 1 to 12 (i.e., 1, 2, 3, 4, 5, 6, 7, 8, 9, 10, 11, or 12); and the dashed line (“ [symbol] ”) represents the point of attachment of the adjuvant. Alternatively, R¹ and the nitrogen atom to which it is attached can form a linking moiety comprising a 5-to 8-membered heterocycle. In some embodiments, subscript n is an integer from 1 to 6 (i.e., 1, 2, 3, 4, 5, or 6). In certain embodiments, subscript n is an integer from 1 to 3 (i.e., 1, 2, or 3).

In some embodiments, the adjuvant (“Adj”) is of formula:

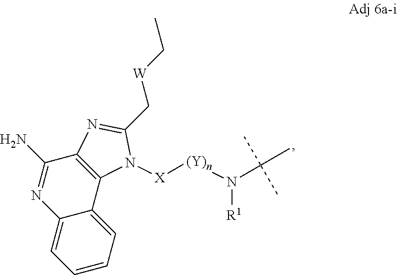

wherein W is selected from the group consisting of O and CH₂; R¹ is selected from H and C₁₋₄ alkyl; each Y is independently CHR², wherein R² is selected from H, OH, and NH₂; subscript n is an integer from 1 to 12 (i.e., 1, 2, 3, 4, 5, 6, 7, 8, 9, 10, 11, or 12); and the dashed line (“ [symbol] ”) represents the point of attachment of the adjuvant. Alternatively, R¹ and the nitrogen atom to which it is attached can form a linking moiety comprising a 5-to 8-membered heterocycle. In some embodiments, subscript n is an integer from 1 to 6 (i.e., 1, 2, 3, 4, 5, or 6). In certain embodiments, subscript n is an integer from 1 to 3 (i.e., 1, 2, or 3).

In some embodiments, the adjuvant (“Adj”) is of formula:

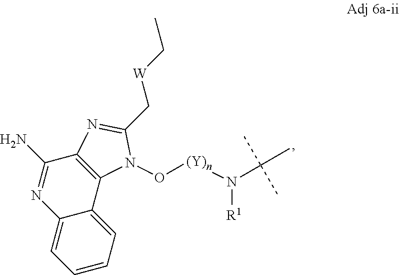

wherein W is selected from the group consisting of O and CH₂; R¹ is selected from H and C₁₋₄ alkyl; each Y is independently CHR², wherein R² is selected from H, OH, and NH₂; subscript n is an integer from 1 to 12 (i.e., 1, 2, 3, 4, 5, 6, 7, 8, 9, 10, 11, or 12); and the dashed line (“ [symbol] ”) represents the point of attachment of the adjuvant. Alternatively, R¹ and the nitrogen atom to which it is attached can form a linking moiety comprising a 5-to 8-membered heterocycle. In some embodiments, subscript n is an integer from 1 to 6 (i.e., 1, 2, 3, 4, 5, or 6). In certain embodiments, subscript n is an integer from 1 to 3 (i.e., 1, 2, or 3).

In some embodiments, the adjuvant (“Adj”) is of formula:

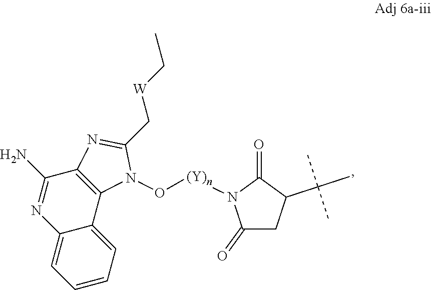

wherein W is selected from the group consisting of O and CH₂; X is selected from O and CH₂; each Y is independently CHR², wherein R² is selected from H, OH, and NH₂; subscript n is an integer from 1 to 12 (i.e., 1, 2, 3, 4, 5, 6, 7, 8, 9, 10, 11, or 12); and the dashed line (“ [symbol] ”) represents the point of attachment of the adjuvant. In some embodiments, subscript n is an integer from 1 to 6 (i.e., 1, 2, 3, 4, 5, or 6). In certain embodiments, subscript n is an integer from 1 to 3 (i.e., 1, 2, or 3).

In some embodiments, the adjuvant (“Adj”) is of formula:

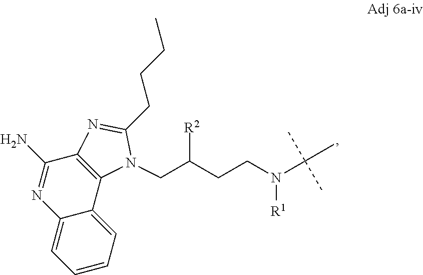

wherein R¹ is selected from H and C₁₋₄ alkyl; R² is selected from H, OH, and NH₂; and the dashed line (“ [symbol] ”) represents the point of attachment of the adjuvant.

In some embodiments, the adjuvant (“Adj”) is of formula:

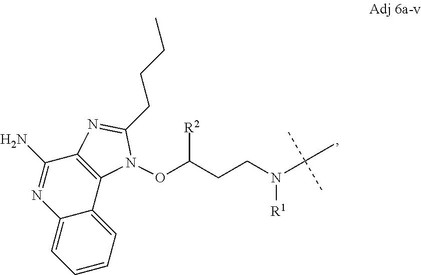

wherein R¹ is selected from H and C₁₋₄ alkyl; R² is selected from H, OH, and NH₂; and the dashed line (“ [symbol] ”) represents the point of attachment of the adjuvant.

In some embodiments, the adjuvant (“Adj”) is of formula:

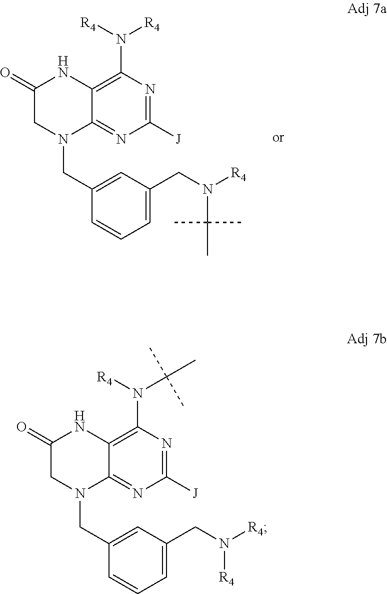

wherein J is hydrogen, OR⁴, or R⁴; each R⁴ independently is hydrogen, or an alkyl, heteroalkyl, cycloalkyl, heterocycloalkyl, aryl, heteroaryl, arylalkyl, or heteroarylalkyl group comprising from 1 to 8 (i.e., 1, 2, 3, 4, 5, 6, 7, or 8) carbon units; and the dashed line (“ [symbol] ”) represents the point of attachment of the adjuvant.

In some embodiments, the adjuvant (“Adj”) is of formula:

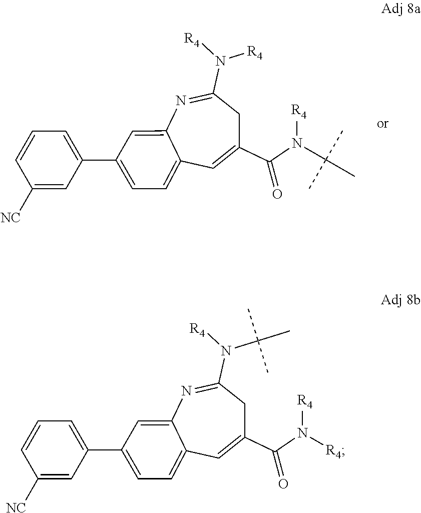

wherein each R⁴ independently is hydrogen, or an alkyl, heteroalkyl, cycloalkyl, heterocycloalkyl, aryl, heteroaryl, arylalkyl, or heteroarylalkyl group comprising from 1 to 8 (i.e., 1, 2, 3, 4, 5, 6, 7, or 8) carbon units and the dashed line (“ [symbol] ”) represents the point of attachment of the adjuvant.

In certain embodiments, the adjuvant (“Adj”) is:

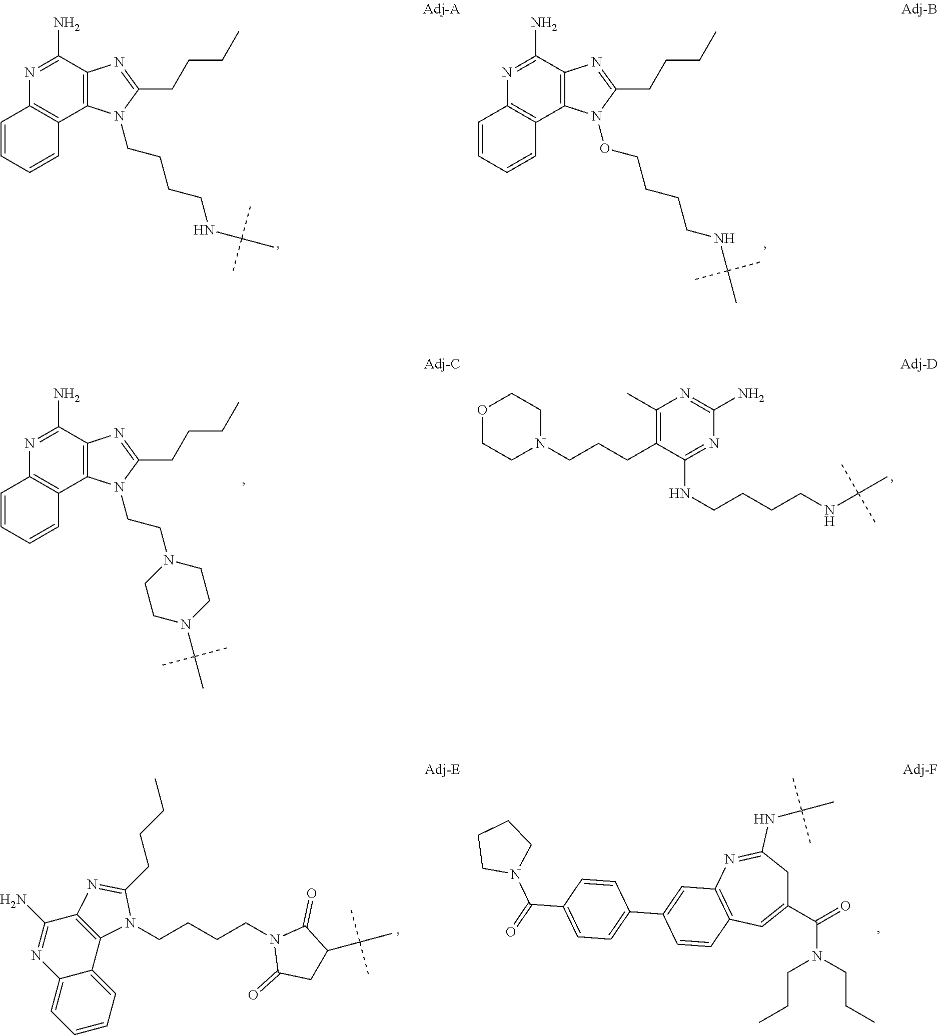

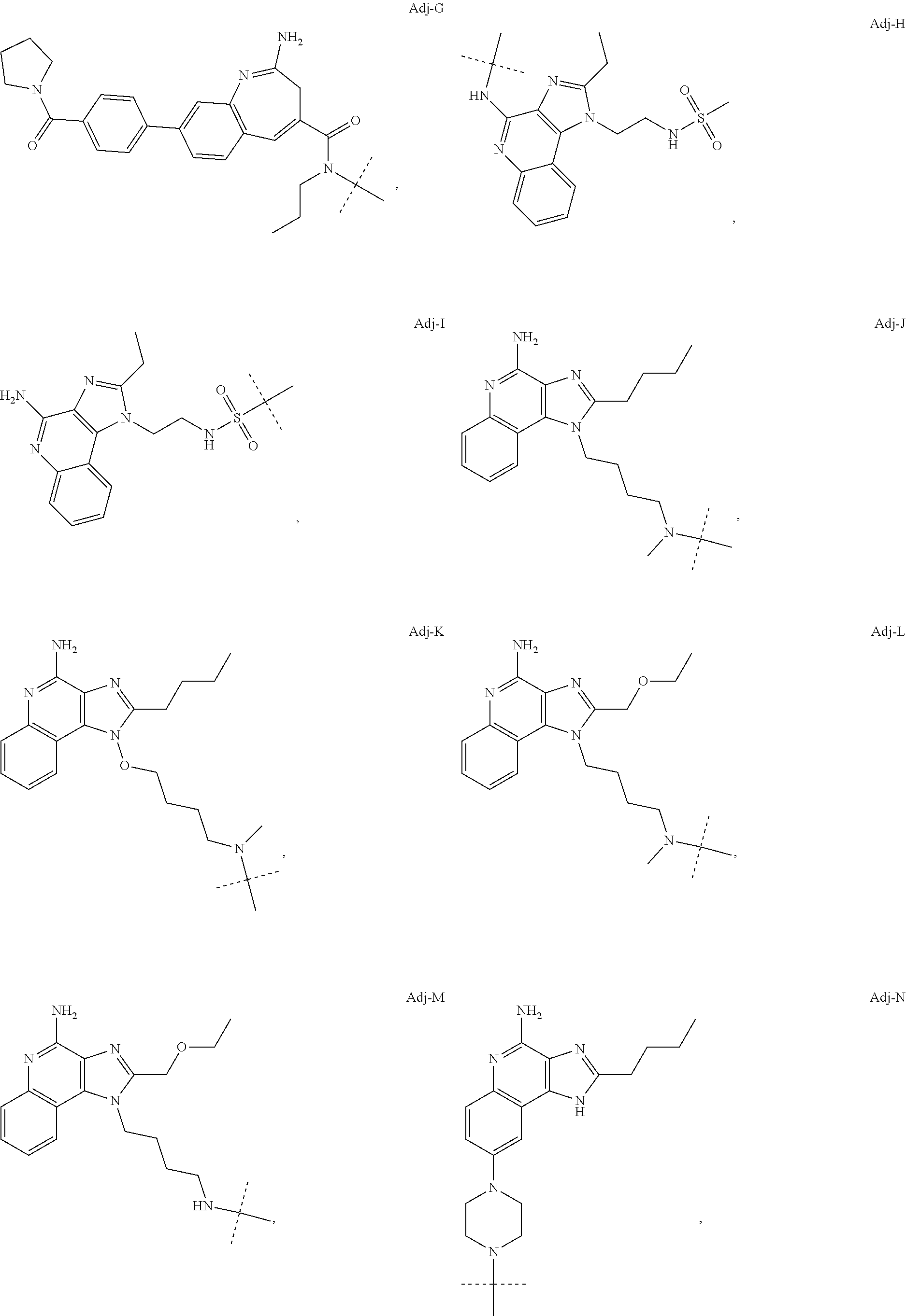

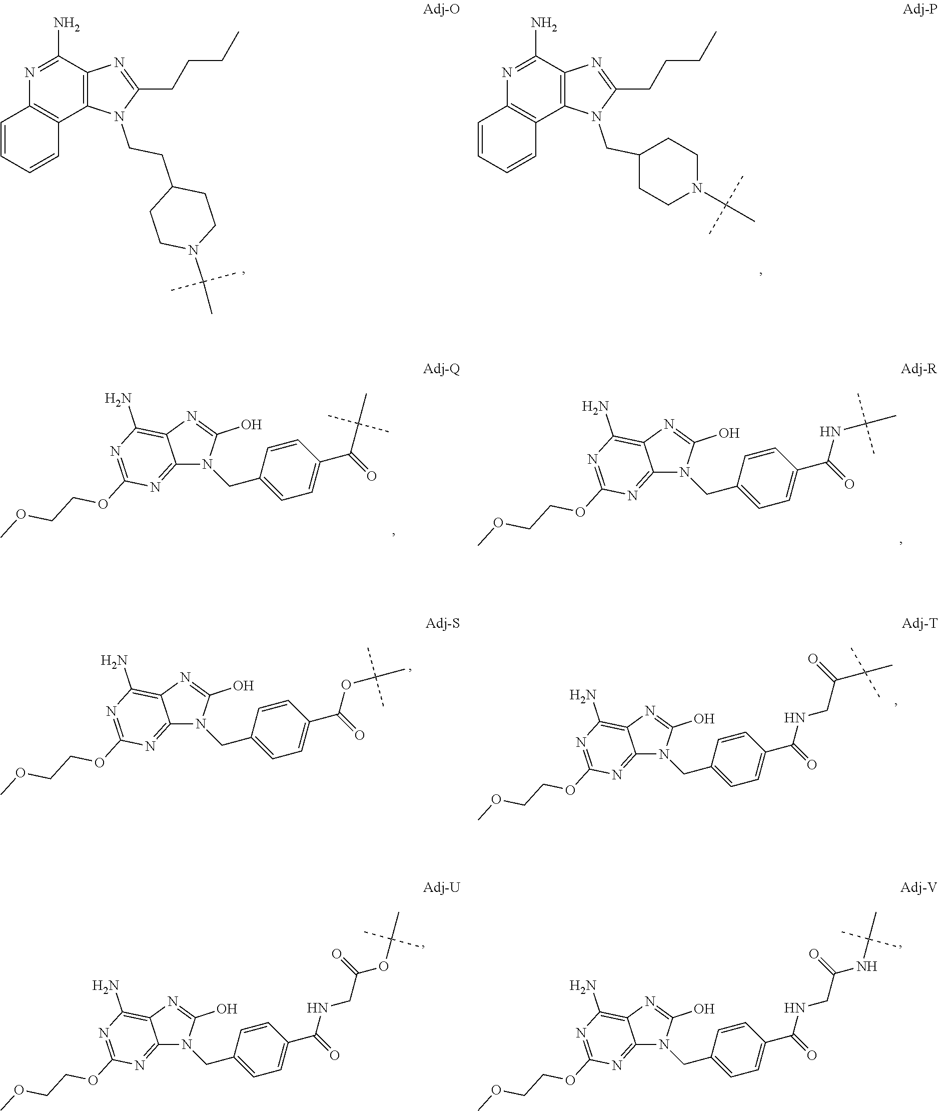

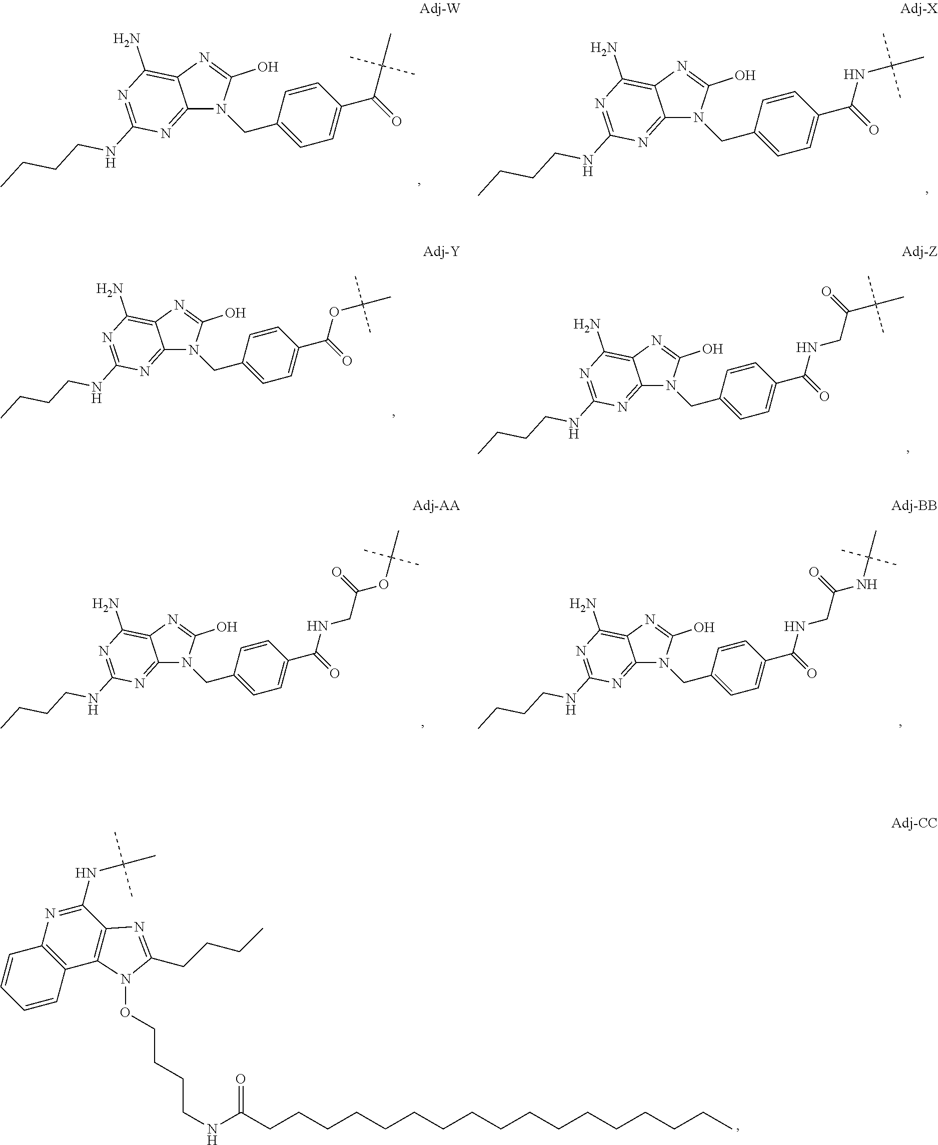

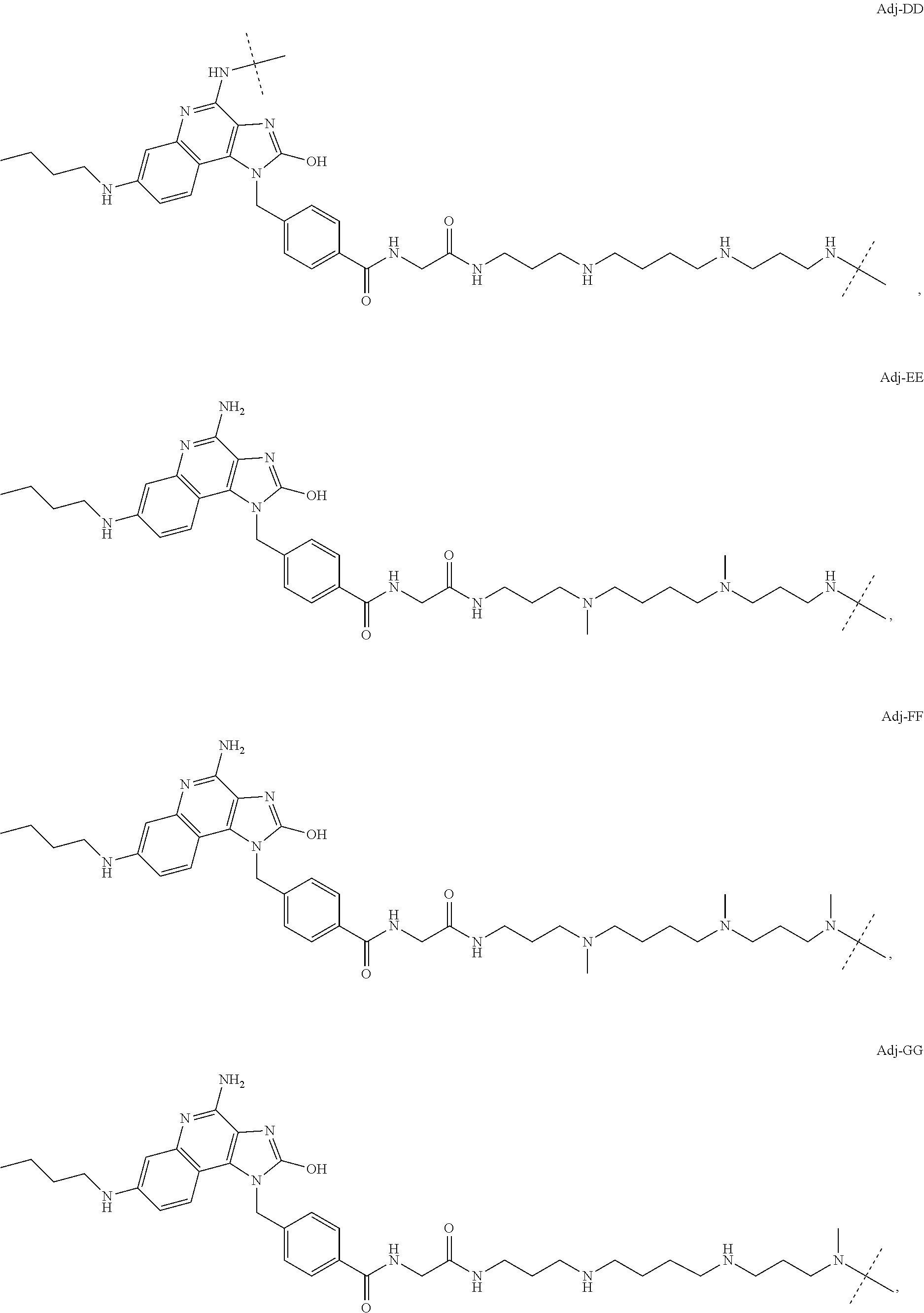

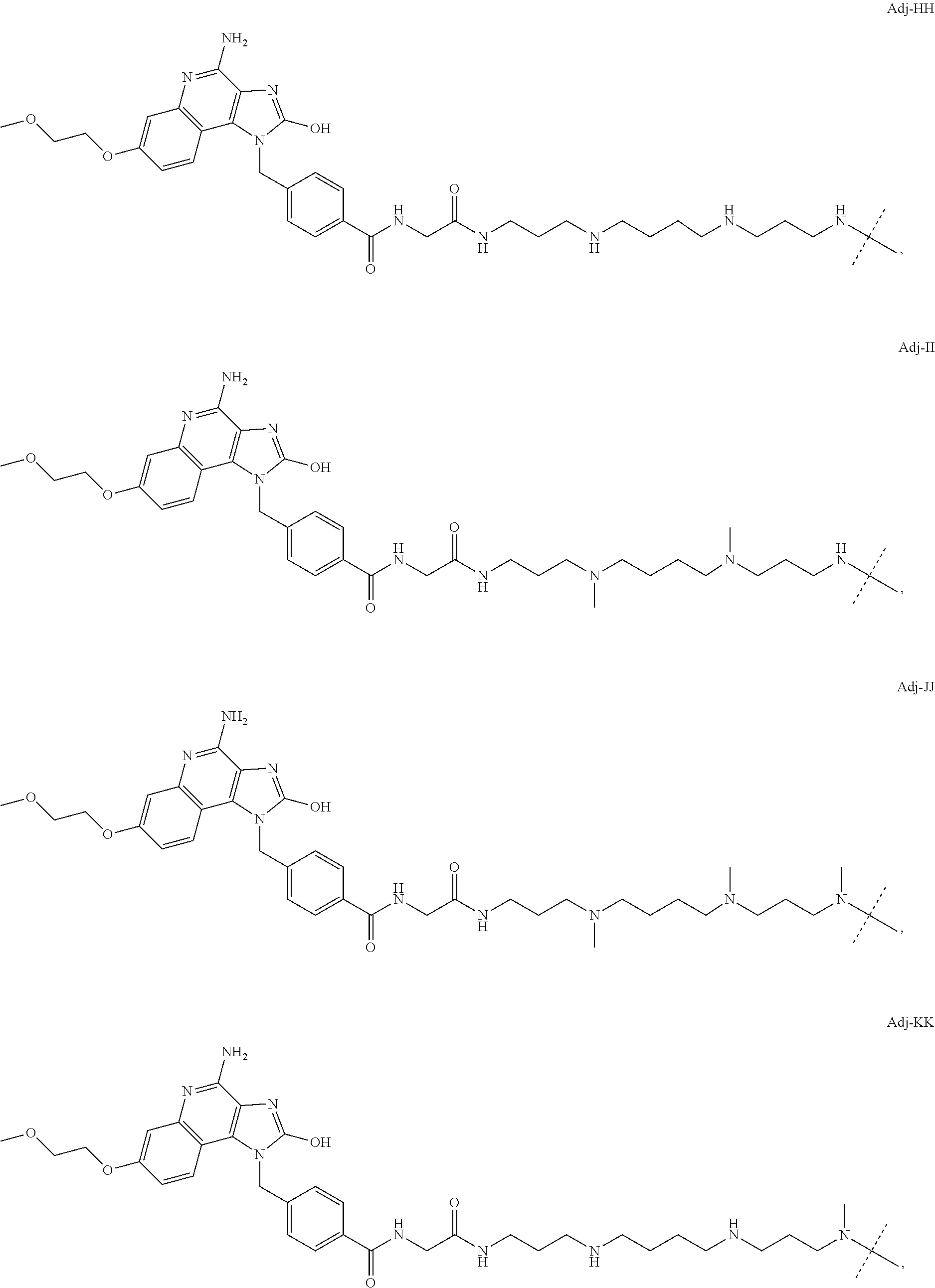

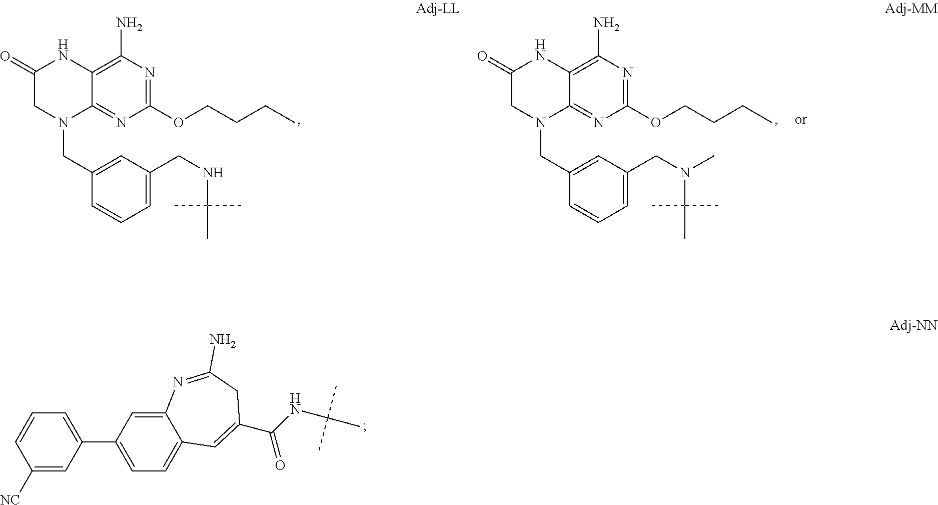

wherein the dashed line (“ [symbol] ”) represents the point of attachment of the adjuvant.

In some embodiments, the adjuvant is not a fluorophore. In some embodiments, the adjuvant is not a radiodiagnostic compound. In some embodiments, the adjuvant is not a radiotherapeutic compound. In some embodiments, the adjuvant is not a tubulin inhibitor. In some embodiments, the adjuvant is not a DNA crosslinker/alkylator. In some embodiments, the adjuvant is not a topoisomerase inhibitor.

### Linkers

The immunoconjugates of the invention containing linking moieties that covalently bond the adjuvants moieties to the antibodies. In some embodiments, the immunoconjugate has a structure according to Formula I:

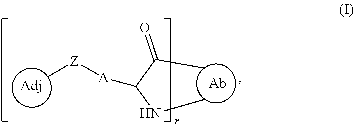

wherein A is an unmodified amino acid sidechain in an antibody or a modified amino acid sidechain in an antibody; Ab is a remainder of an antibody containing amino acid side chain A; Z is a linking moiety; Adj is an adjuvant moiety; and subscript r is an integer from 1 to 10.

In a related aspect, the invention provides a composition comprising a plurality of immunoconjugates as described herein. In some embodiments, the average number of adjuvant moieties per immunoconjugate ranges from about 1 to about 10 (e.g., from about 1 to about 4).

The adjuvant moieties in the conjugates can be covalently bonded to the antibodies using various chemistries for protein modification, and that the linking moieties described above result from the reaction of protein functional groups (i.e., amino acid side chains), with reagents having reactive linker groups. A wide variety of such reagents are known in the art. Examples of such reagents include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C-H) insertion); pentafluorophenyl (PFP) esters (amine reactive); tetrafluorophenyl (TFP) esters (amine reactive); imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Further reagents include but are not limited to those described in Hermanson, Bioconjugate Techniques, 2nd Edition, Academic Press, 2008.

The linker can have any suitable length such that when the linker is covalently bound to the antibody construct and the adjuvant moiety, the function of the antibody construct and the adjuvant moiety is maintained. The linker can have a length of about 3 Å or more, for example, about 4 Å or more, about 5 Å or more, about 6 Å or more, about 7 Å or more, about 8 Å or more, about 9 Å or more, or about 10 Å or more. Alternatively, or in addition to, the linker can have a length of about 50 Å or less, for example, about 45 Å or less, about 40 Å or less, about 35 Å or less, about 30 Å or less, about 25 Å or less, about 20 Å or less, or about 15 Å or less. Thus, the linker can have a length bounded by any two of the aforementioned endpoints. The linker can have a length from about 3 Å to about 50 Å, for example, from about 3 Å to about 45 Å, from about 3 Å to about 40 Å, from about 3 Å to about 35 Å, from about 3 Å to about 30 Å, from about 3 Å to about 25 Å, from about 3 Å to about 20 Å, from about 3 Å to about 15 Å, from about 5 Å to about 50 Å, from about 5 Å to about 25 Å, from about 5 Å to about 20 Å, from about 10 Å to about 50 Å, from about 10 Å to about 20 Å, from about 5 Å to about 30 Å, or from about 5 Å to about 15 Å. In certain embodiments, the linker has a length from about 3 Å to about 20 Å.

In some embodiments, the linker is non-cleavable under physiological conditions.

In some embodiments, the immunoconjugate has a structure according to Formula II:

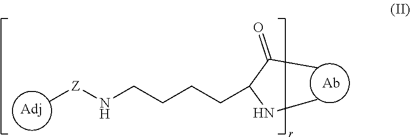

wherein

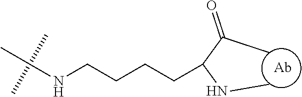

is an antibody with residue

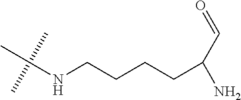

representing a lysine residue of the antibody, wherein “ [symbol] ” represents a point of attachment to Z; Adj is an adjuvant; subscript r is an integer from 1 to 10; and Z is a divalent linking moiety having an ethylene glycol group or a glycine residue. Z preferably is bonded to the adjuvant via an amide bond, a C—N single bond, a C—O single bond, or a C—C single bond, and to the antibody via an amide bond or a C—N single bond. In some embodiments, Z is bonded to a nitrogen group of the adjuvant and a nitrogen group of the antibody. In such embodiments, Z is bonded to adjacent nitrogen groups via amide bonds, C—N single bonds, or a combination thereof.

In certain embodiments, the invention provides immunoconjugate having a structure according to Formula IIa:

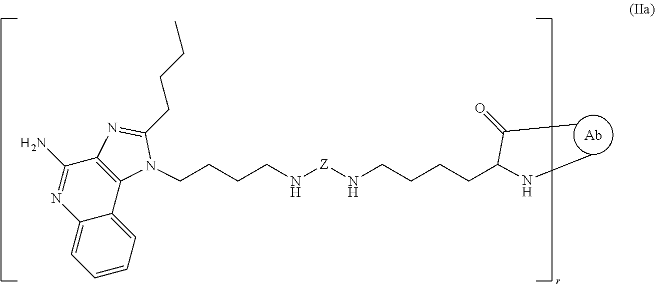

wherein:

Ab is an antibody;

subscript r is an integer from 1 to 10; and

Z is a divalent linking moiety comprising an ethylene glycol group or a glycine residue. Z preferably is bonded to the adjuvant via an amide bond, a C—N single bond, a C—O single bond, or a C—C single bond, and to the antibody via an amide bond or a C—N single bond. In some embodiments, Z is bonded to a nitrogen group of the adjuvant and a nitrogen group of the antibody. In such embodiments, Z is bonded to adjacent nitrogen groups via amide bonds, C—N single bonds, or a combination thereof.

In some embodiments, Z comprises a poly(ethylene glycol) group. In certain embodiments, Z comprises at least 2 ethylene glycol groups (e.g., at least 3 ethylene glycol groups, at least 4 ethylene glycol groups, at least 5 ethylene glycol groups, at least 6 ethylene glycol groups, at least 7 ethylene glycol groups, at least 8 ethylene glycol groups, at least 9 ethylene glycol groups, at least 10 ethylene glycol groups, at least 11 ethylene glycol groups, at least 12 ethylene glycol groups, at least 13 ethylene glycol groups, at least 14 ethylene glycol groups, at least 15 ethylene glycol groups, at least 16 ethylene glycol groups, at least 17 ethylene glycol groups, at least 18 ethylene glycol groups, at least 19 ethylene glycol groups, at least 20 ethylene glycol groups, at least 21 ethylene glycol groups, at least 22 ethylene glycol groups, at least 23 ethylene glycol groups, at least 24 ethylene glycol groups, or at least 25 ethylene glycol groups. In certain embodiments, Z comprises a di(ethylene glycol) group, a tri(ethylene glycol) group, or a tetra(ethylene glycol) group, 5 ethylene glycol groups, 6 ethylene glycol groups, 8 ethylene glycol groups, 12 ethylene glycol groups, 24 ethylene glycol groups, or 25 ethylene glycol groups.

In some embodiments, Z comprises a glycine residue. In certain embodiments, Z comprises at least 2 glycine residues (e.g., at least 3 glycine residues, at least 4 glycine residues, at least 5 glycine residues, at least 6 glycine residues, at least 7 glycine residues, at least 8 glycine residues, at least 9 glycine residues, at least 10 glycine residues, at least 11 glycine residues, at least 12 glycine residues, at least 13 glycine residues, at least 14 glycine residues, at least 15 glycine residues, at least 16 glycine residues, at least 17 glycine residues, at least 18 glycine residues, at least 19 glycine residues, at least 20 glycine residues, at least 21 glycine residues, at least 22 glycine residues, at least 23 glycine residues, at least 24 glycine residues, or at least 25 glycine residues. In certain embodiments, Z comprises 2 glycine residues, 3 glycine residues, 4 glycine residues, 5 glycine residues, 6 glycine residues, 8 glycine residues, 12 glycine residues, 24 glycine residues, or 25 glycine residues.

In some embodiments, Z further comprises a divalent cyclohexylene group.

In some embodiments, the immunoconjugate has a structure according to Formula IIIa:

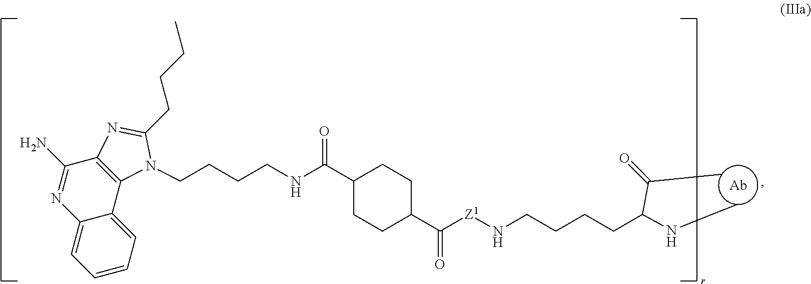

wherein

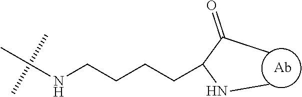

is an antibody with residue

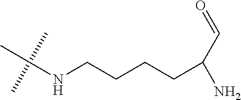

representing a lysine residue of the antibody, wherein “ [symbol] ” represents a point of attachment to Z¹, wherein Z¹ comprises at least one ethylene glycol group or at least one glycine residue.

In some embodiments, the immunoconjugate has a structure according to Formula IIIb:

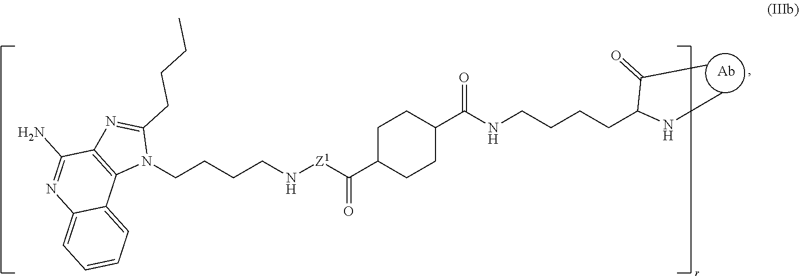

wherein

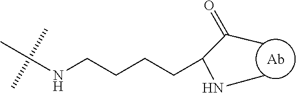

is an antibody with residue

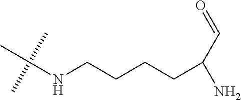

representing a lysine residue of the antibody, wherein “ [symbol] ” represents a point of attachment to Z¹, wherein Z¹ comprises at least one ethylene glycol group or at least one glycine residue, wherein Z¹ comprises at least one ethylene glycol group or at least one glycine residue.

In some embodiments, the immunoconjugate has a structure according to Formula IV:

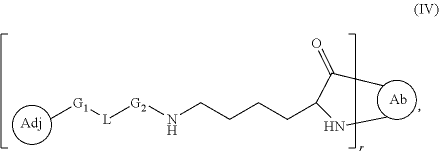

or a pharmaceutically acceptable salt thereof, wherein

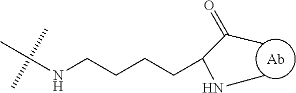

is an antibody with residue

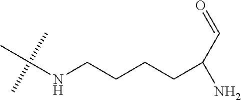

representing a lysine residue of the antibody, wherein “ [symbol] ” represents a point of attachment to G₂, Adj is an adjuvant, G₁ is CH₂, C═O, or a bond, G₂ is CH₂, C═O, or a bond, L is a linker, and subscript r is an integer from 1 to 10 (i.e., 1, 2, 3, 4, 5, 6, 7, 8, 9, or 10). In certain embodiments of the immunoconjugate of Formula IV, the antibody does not contain a thiol-modified lysine sidechain.

In some embodiments, L is selected from:

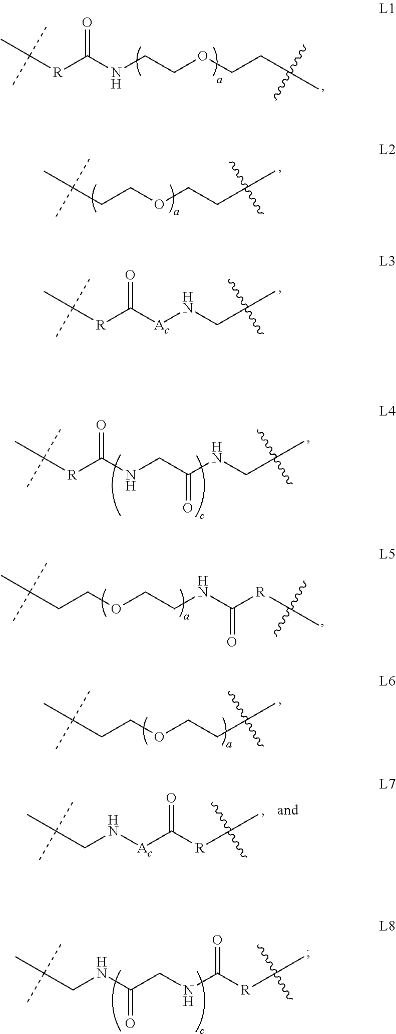

wherein R is optionally present and is a linear or branched, cyclic or straight, saturated or unsaturated alkyl, heteroalkyl, aryl, or heteroaryl chain comprising from 1 to 8 carbon units; a is an integer from 1 to 40; each A is independently selected from any amino acid; subscript c is an integer from 1 to 25; the dashed line (“ [symbol] ”) represents the point of attachment to G₁; and the wavy line (“ [symbol] ”) represents the point of attachment to G₂. In certain embodiments, a is an integer from 2 to 25. In certain embodiments, c is an integer from 2 to 8.

In some embodiments, the immunoconjugate has a structure according to Formula IVa:

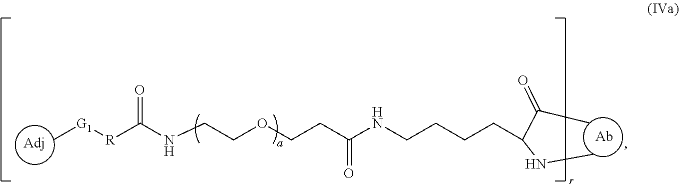

or a pharmaceutically acceptable salt thereof, wherein Ab is as defined herein; Adj is an adjuvant; G₁ is CH₂, C═O, or a bond; R is optionally present and is a linear or branched, cyclic or straight, saturated or unsaturated alkyl, heteroalkyl, aryl, or heteroaryl chain comprising from 1 to 8 carbon units; subscript a is an integer from 1 to 40; and subscript r is an integer from 1 to 10 (i.e., 1, 2, 3, 4, 5, 6, 7, 8, 9, or 10). In certain embodiments, a is an integer from 2 to 25.

In some embodiments, the immunoconjugate has a structure according to Formula IVb:

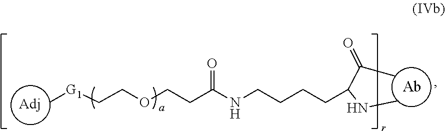

or a pharmaceutically acceptable salt thereof, wherein Ab is as defined herein; Adj is an adjuvant; G₁ is CH₂, C═O, or a bond; subscript a is an integer from 1 to 40; and subscript r is an integer from 1 to 10 (i.e., 1, 2, 3, 4, 5, 6, 7, 8, 9, or 10). In certain embodiments, a is an integer from 2 to 25.

In some embodiments, the immunoconjugate has a structure according to Formula IVc:

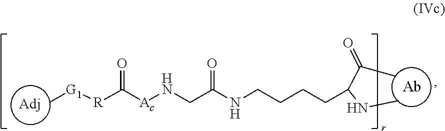

or a pharmaceutically acceptable salt thereof, wherein Ab is as defined herein; Adj is an adjuvant; G₁ is CH₂, C═O, or a bond; R is optionally present and is a linear or branched, cyclic or straight, saturated or unsaturated alkyl, heteroalkyl, aryl, or heteroaryl chain comprising from 1 to 8 carbon units; each A is independently selected from any amino acid; subscript c is an integer from 1 to 25; and subscript r is an integer from 1 to 10 (i.e., 1, 2, 3, 4, 5, 6, 7, 8, 9, or 10). In certain embodiments, c is an integer from 2 to 8.

In some embodiments, the immunoconjugate has a structure according to Formula IVd:

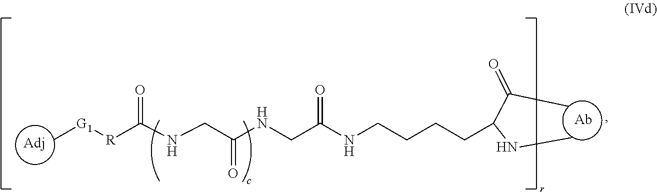

or a pharmaceutically acceptable salt thereof, wherein Ab is as defined herein; Adj is an adjuvant; G₁ is CH₂, C═O, or a bond; R is optionally present and is a linear or branched, cyclic or straight, saturated or unsaturated alkyl, heteroalkyl, aryl, or heteroaryl chain comprising from 1 to 8 carbon units; subscript c is an integer from 1 to 25; and subscript r is an integer from 1 to 10 (i.e., 1, 2, 3, 4, 5, 6, 7, 8, 9, or 10). In certain embodiments, c is an integer from 2 to 8.

In some embodiments, the immunoconjugate has a structure according to Formula IVe:

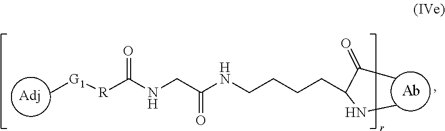

or a pharmaceutically acceptable salt thereof, wherein Ab is as defined herein; Adj is an adjuvant; G₁ is CH₂, C═O, or a bond; R is optionally present and is a linear or branched, cyclic or straight, saturated or unsaturated alkyl, heteroalkyl, aryl, or heteroaryl chain comprising from 1 to 8 carbon units; and subscript r is an integer from 1 to 10 (i.e., 1, 2, 3, 4, 5, 6, 7, 8, 9, or 10).

Accordingly, the immunoconjugate can have a structure according to Formula Va-Formula Vff:

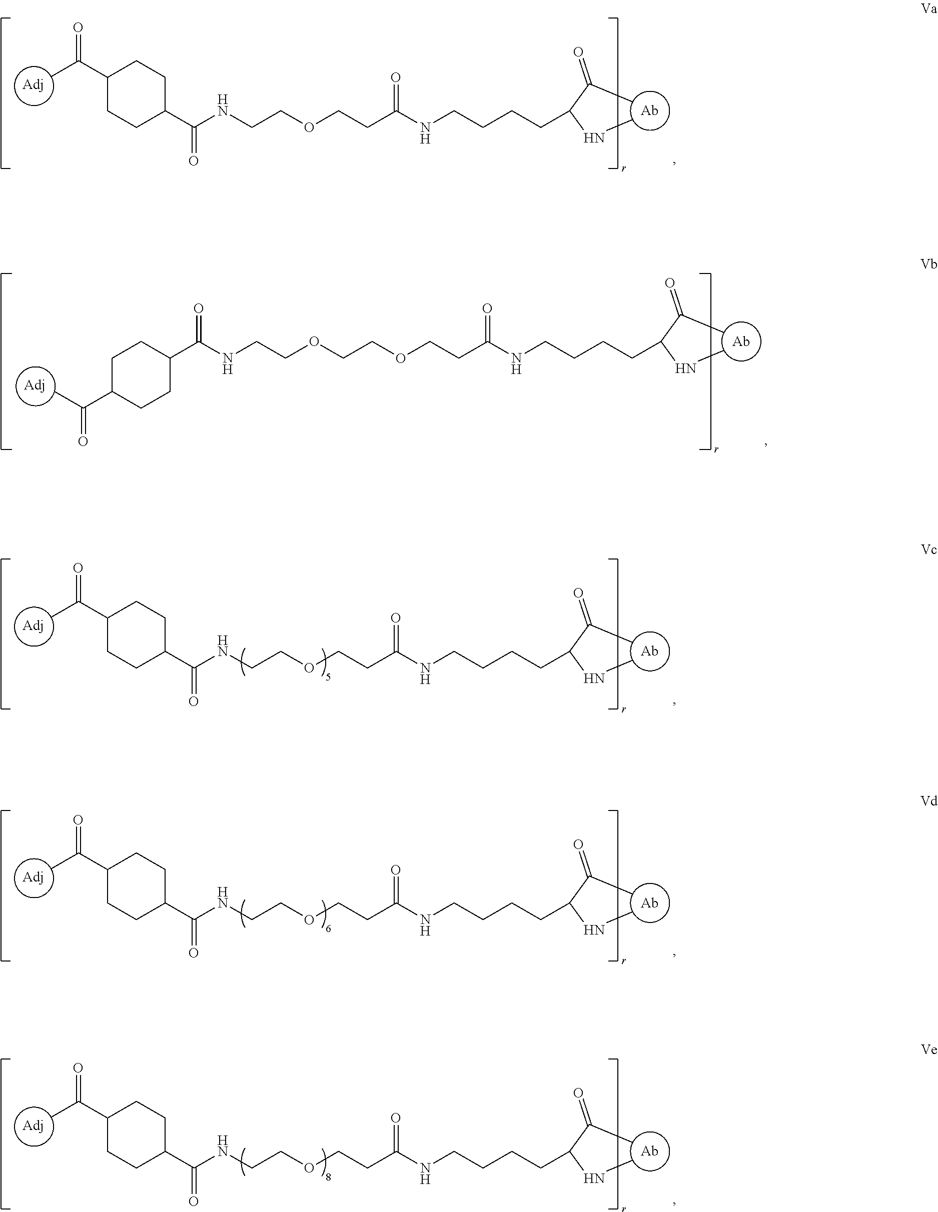

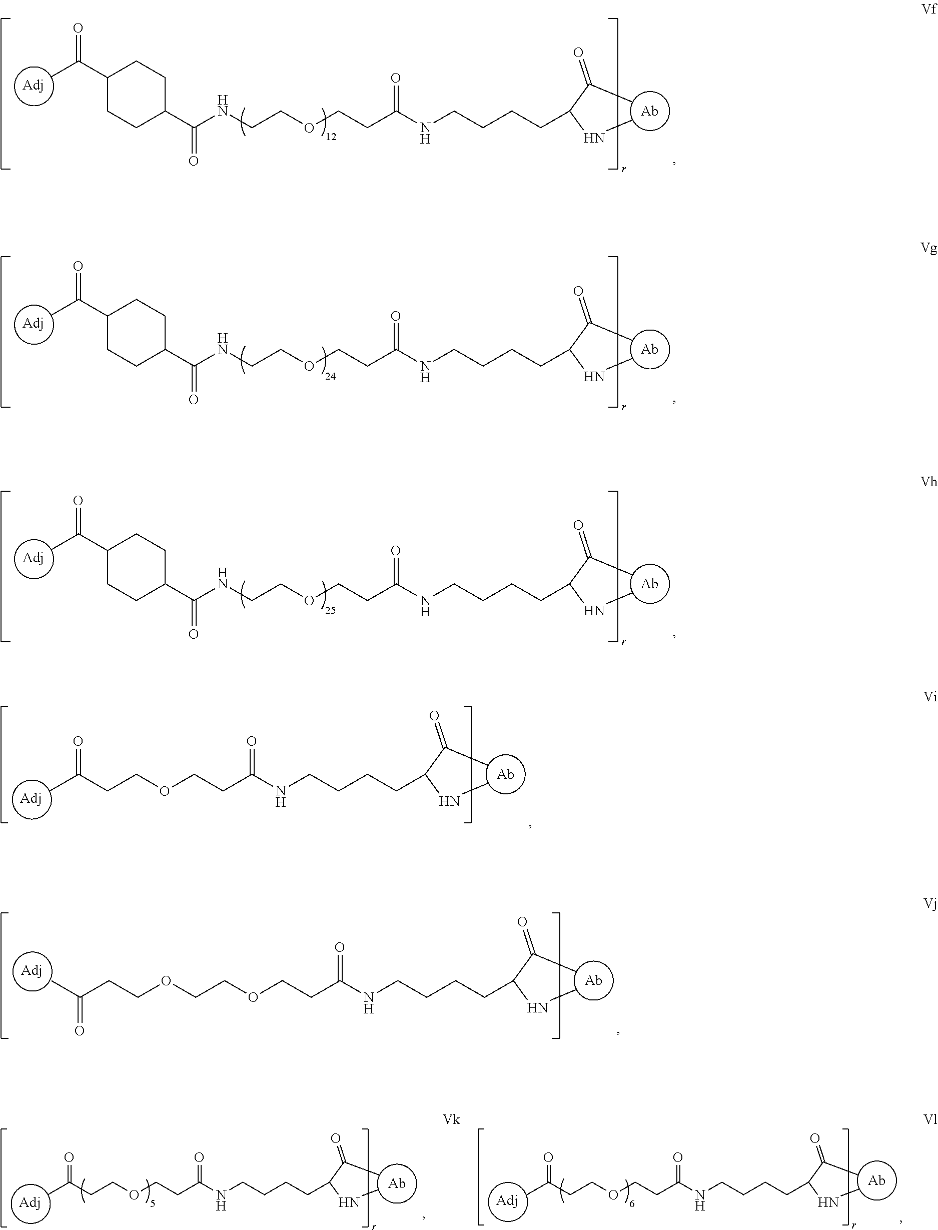

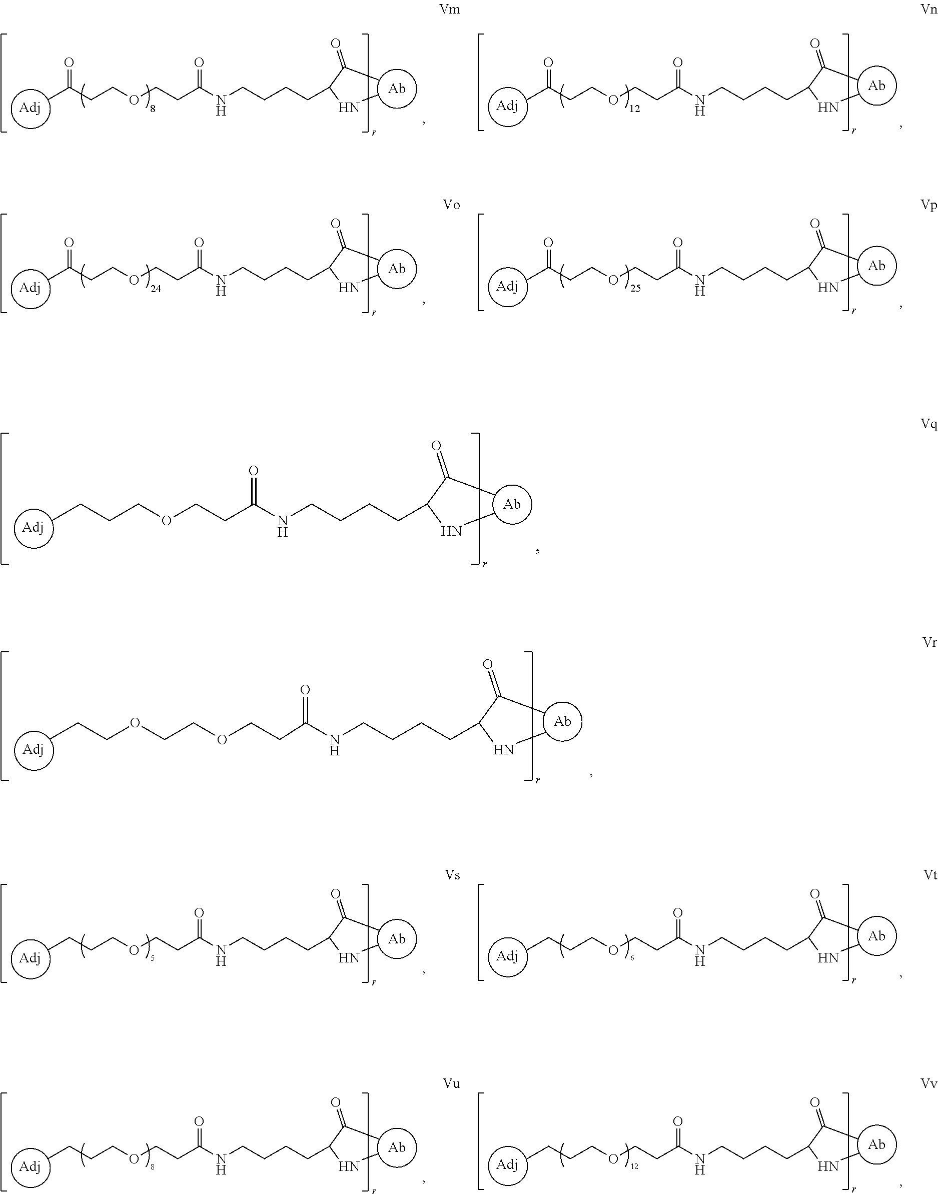

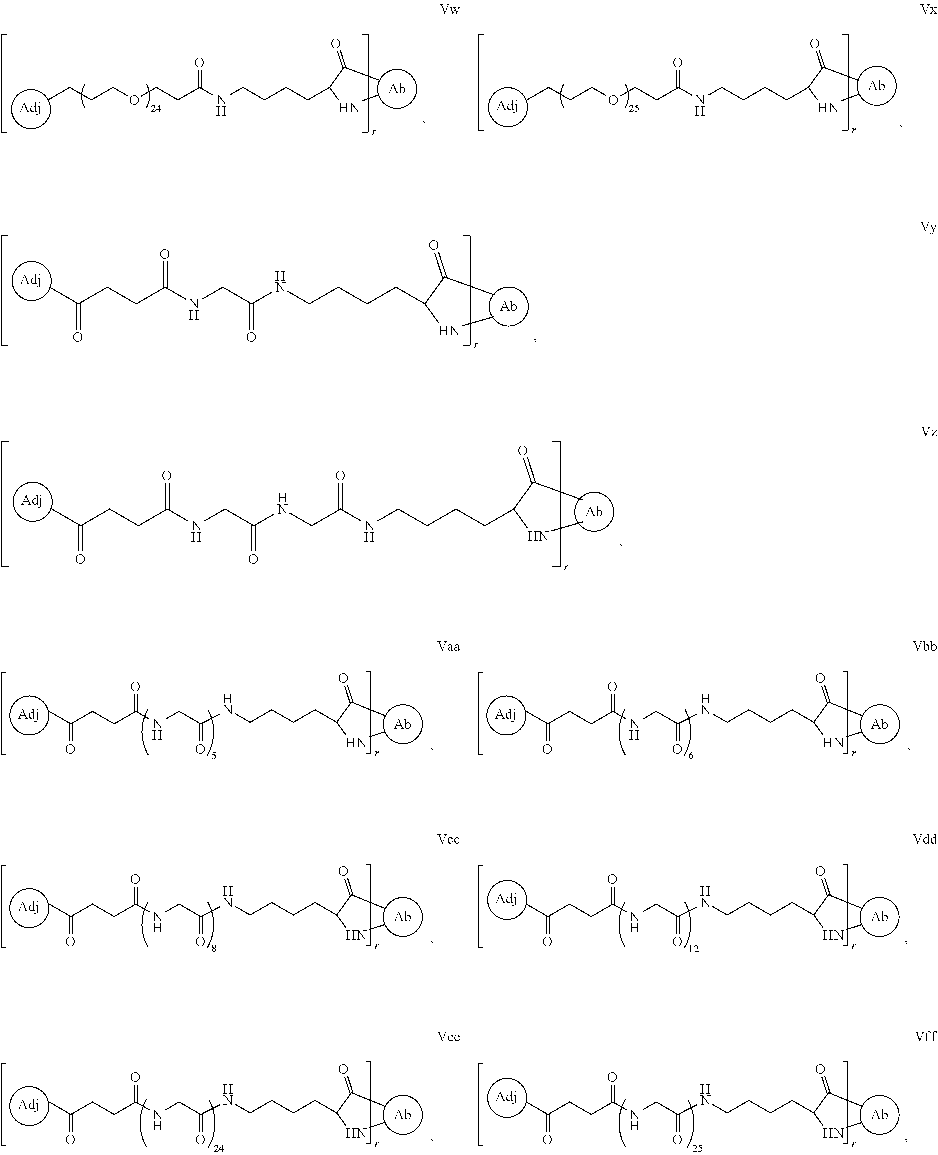

or a pharmaceutically acceptable salt thereof, wherein Ab is as defined herein; Adj is an adjuvant; and subscript r is an integer from 1 to 10 (i.e., 1, 2, 3, 4, 5, 6, 7, 8, 9, or 10). In certain embodiments, subscript r is an integer from 1 to 4 (i.e., 1, 2, 3, or 4).

For example, the immunoconjugate can have a structure according to Formula VIa-VIj:

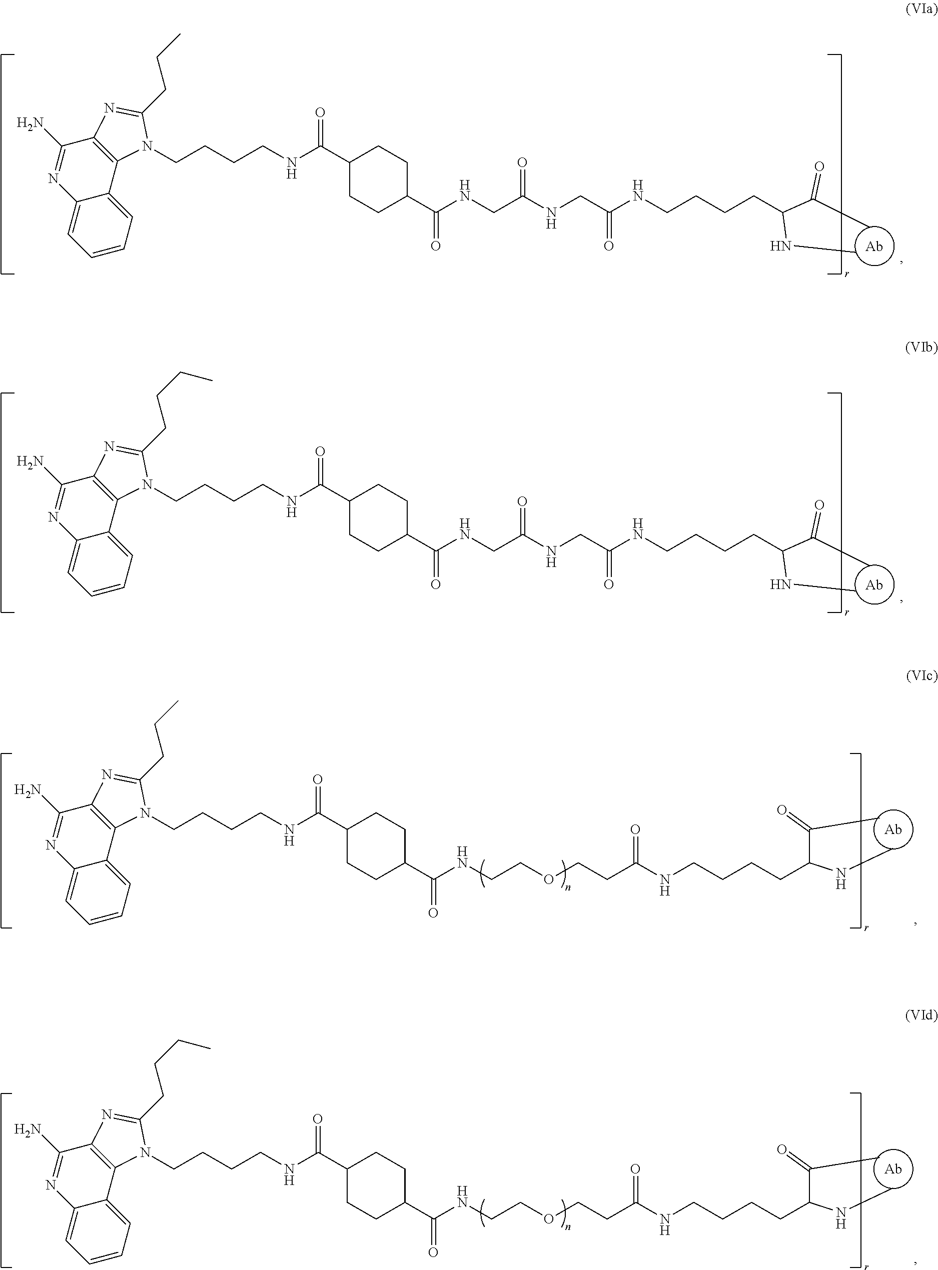

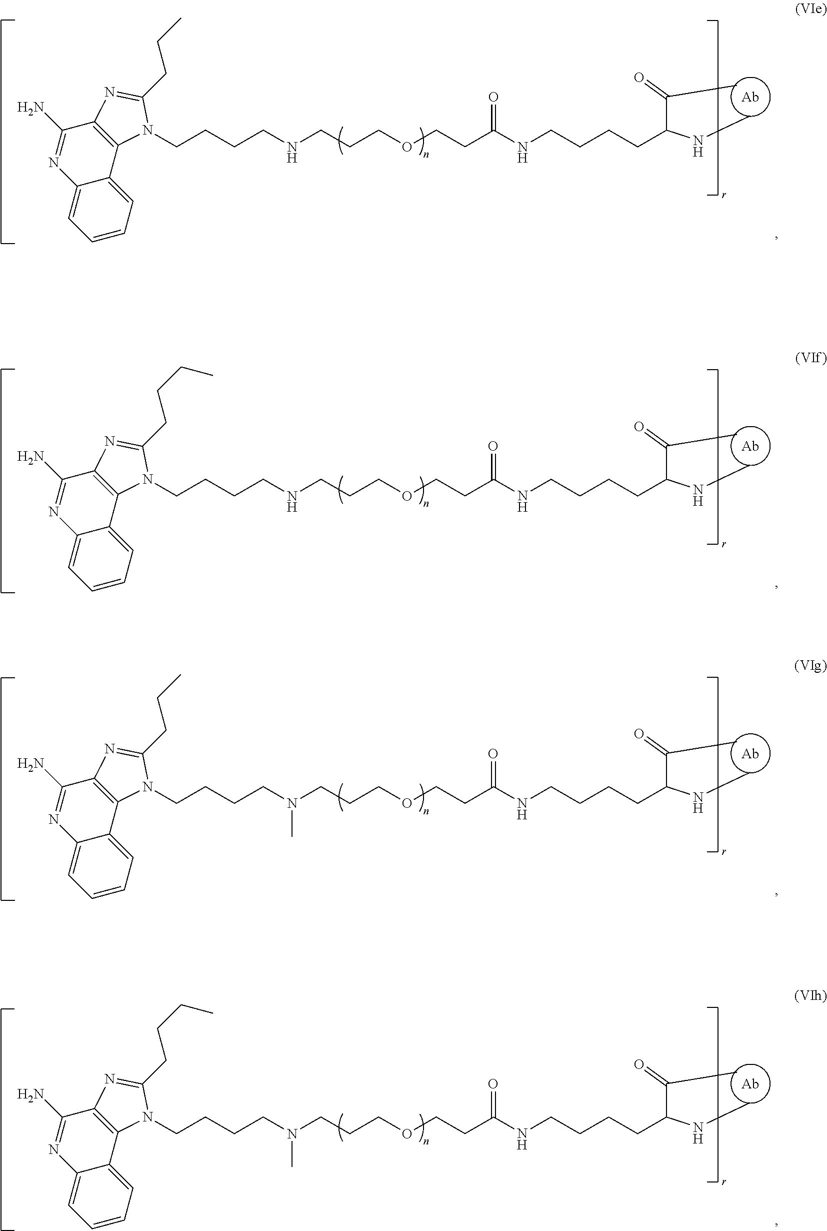

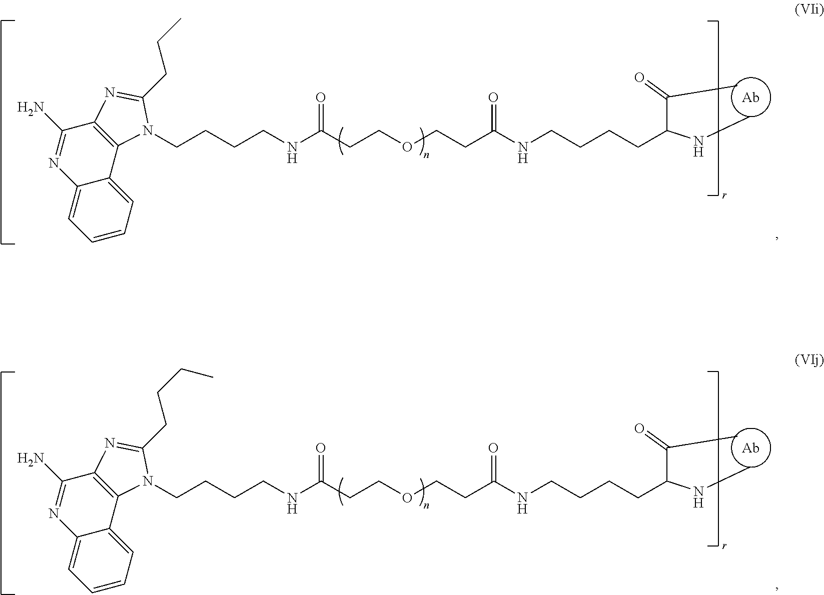

wherein n is an integer ranging from 1 to 40 and r is an integer from 1 to 10 (i.e., 1, 2, 3, 4, 5, 6, 7, 8, 9, or 10). In certain embodiments, subscript r is an integer from 1 to 4 (i.e., 1, 2, 3, or 4). In some embodiments, n is an integer from 2 to 25. In certain embodiments, n is an integer ranging from 2 to 8.

In a second aspect, the invention provides an improved method for producing an immunoconjugate of Formula IV from one or more compounds of Formula VII and an antibody of Formula VIII, the method comprising the step of:

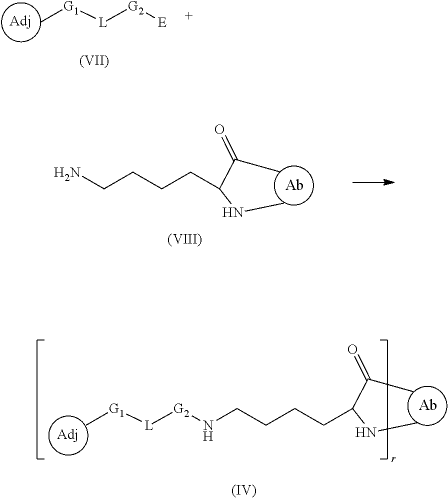

wherein

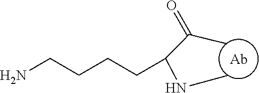

is an antibody with residue

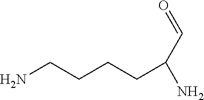

representing a lysine residue of the antibody, Adj is an adjuvant; G₁ is CH₂, C═O, or a bond; G₂ is CH₂, C═O, or a bond; L is a linker; E is an ester; and subscript r is an integer from 1 to 10 (i.e., 1, 2, 3, 4, 5, 6, 7, 8, 9, or 10). In certain embodiments, subscript r is an integer from 1 to 4 (i.e., 1, 2, 3, or 4).

Any suitable linker can be used provided it can be bound to the antibody (compound of Formula VII) through an ester. For example, the linker (“L”) can have the following formula

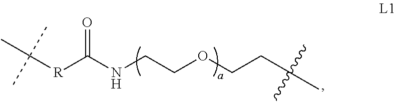

wherein R is optionally present and is a linear or branched, cyclic or straight, saturated or unsaturated alkyl, heteroalkyl, aryl, or heteroaryl chain comprising from 1 to 8 (i.e., 1, 2, 3, 4, 5, 6, 7, or 8) carbon units; subscript a is an integer from 1 to 40; the dashed line (“ [symbol] ”) represents the point of attachment to G₁; and the wavy line (“ [symbol] ”) represents the point of attachment to G₂. In some embodiments, subscript a is an integer from 1 to 25. In some embodiments, subscript a is an integer from 2 to 25. In some embodiments, subscript a is an integer from 2 to 8. In certain embodiments, R is present and is a linear or branched, cyclic or straight, saturated or unsaturated alkyl, heteroalkyl, aryl, or heteroaryl chain comprising from 1 to 8 (i.e., 1, 2, 3, 4, 5, 6, 7, or 8) carbon units.

The linker (“L”) can have the following formula

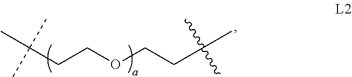

wherein subscript a is an integer from 1 to 40; the dashed line (“ [symbol] ”) represents the point of attachment to G₁, and the wavy line (“ [symbol] ”) represents the point of attachment to G₂. In some embodiments, subscript a is an integer from 1 to 25. In some embodiments, subscript a is an integer from 2 to 25. In some embodiments, subscript a is an integer from 2 to 8.

The linker (“L”) can also have the following formula

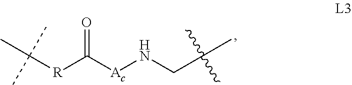

wherein R is optionally present and is a linear or branched, cyclic or straight, saturated or unsaturated alkyl, heteroalkyl, aryl, or heteroaryl chain comprising from 1 to 8 (i.e., 1, 2, 3, 4, 5, 6, 7, or 8) carbon units; each A is independently selected from any amino acid; subscript c is an integer from 1 to 25; the dashed line (“ [symbol] ”) represents the point of attachment to G₁; and the wavy line (“ [symbol] ”) represents the point of attachment to G₂. In some embodiments, subscript c is an integer from 2 to 25. In some embodiments, subscript c is an integer from 1 to 8. In some embodiments, subscript c is an integer from 2 to 8. In certain embodiments, R is present and is a linear or branched, cyclic or straight, saturated or unsaturated alkyl, heteroalkyl, aryl, or heteroaryl chain comprising from 1 to 8 (i.e., 1, 2, 3, 4, 5, 6, 7, or 8) carbon units.

The linker (“L”) can also have the following formula

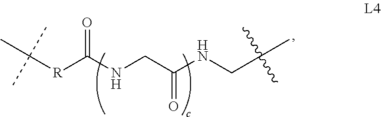

wherein R is optionally present and is a linear or branched, cyclic or straight, saturated or unsaturated alkyl, heteroalkyl, aryl, or heteroaryl chain comprising from 1 to 8 (i.e., 1, 2, 3, 4, 5, 6, 7, or 8) carbon units; subscript c is an integer from 1 to 25; the dashed line (“ [symbol] ”) represents the point of attachment to G₁; and the wavy line (“ [symbol] ”) represents the point of attachment to G₂. In some embodiments, subscript c is an integer from 2 to 25. In some embodiments, c is an integer from 1 to 8. In some embodiments, c is an integer from 2 to 8. In certain embodiments, R is present and is a linear or branched, cyclic or straight, saturated or unsaturated alkyl, heteroalkyl, aryl, or heteroaryl chain comprising from 1 to 8 (i.e., 1, 2, 3, 4, 5, 6, 7, or 8) carbon units.

The linker (“L”) can have the following formula

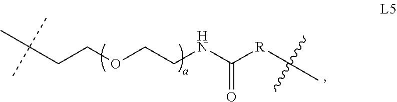

wherein R is optionally present and is a linear or branched, cyclic or straight, saturated or unsaturated alkyl, heteroalkyl, aryl, or heteroaryl chain comprising from 1 to 8 (i.e., 1, 2, 3, 4, 5, 6, 7, or 8) carbon units; subscript a is an integer from 1 to 40; the dashed line (“ [symbol] ”) represents the point of attachment to G₁; and the wavy line (“ [symbol] ”) represents the point of attachment to G₂. In some embodiments, subscript a is an integer from 1 to 25. In some embodiments, subscript a is an integer from 2 to 25. In some embodiments, subscript a is an integer from 2 to 8. In certain embodiments, R is present and is a linear or branched, cyclic or straight, saturated or unsaturated alkyl, heteroalkyl, aryl, or heteroaryl chain comprising from 1 to 8 (i.e., 1, 2, 3, 4, 5, 6, 7, or 8) carbon units.

The linker (“L”) can have the following formula

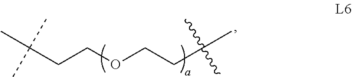

wherein subscript a is an integer from 1 to 40; the dashed line (“ [symbol] ”) represents the point of attachment to G₁, and the wavy line (“ [symbol] ”) represents the point of attachment to G₂. In some embodiments, subscript a is an integer from 1 to 25. In some embodiments, subscript a is an integer from 2 to 25. In some embodiments, subscript a is an integer from 2 to 8.

The linker (“L”) can also have the following formula

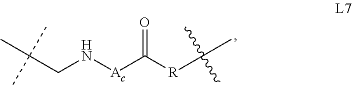

wherein R is optionally present and is a linear or branched, cyclic or straight, saturated or unsaturated alkyl, heteroalkyl, aryl, or heteroaryl chain comprising from 1 to 8 (i.e., 1, 2, 3, 4, 5, 6, 7, or 8) carbon units; each A is independently selected from any amino acid; subscript c is an integer from 1 to 25; the dashed line (“ [symbol] ”) represents the point of attachment to G₁; and the wavy line (“ [symbol] ”) represents the point of attachment to G₂. In some embodiments, subscript c is an integer from 2 to 25. In some embodiments, subscript c is an integer from 1 to 8. In some embodiments, subscript c is an integer from 2 to 8. In certain embodiments, R is present and is a linear or branched, cyclic or straight, saturated or unsaturated alkyl, heteroalkyl, aryl, or heteroaryl chain comprising from 1 to 8 (i.e., 1, 2, 3, 4, 5, 6, 7, or 8) carbon units.

The linker (“L”) can also have the following formula

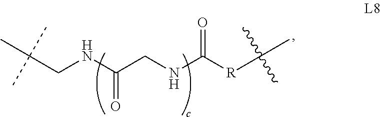

wherein R is optionally present and is a linear or branched, cyclic or straight, saturated or unsaturated alkyl, heteroalkyl, aryl, or heteroaryl chain comprising from 1 to 8 (i.e., 1, 2, 3, 4, 5, 6, 7, or 8) carbon units; subscript c is an integer from 1 to 20; the dashed line (“ [symbol] ”) represents the point of attachment to G₁; and the wavy line (“ [symbol] ”) represents the point of attachment to G₂. In some embodiments, subscript c is an integer from 2 to 25. In some embodiments, subscript c is an integer from 1 to 8. In some embodiments, subscript c is an integer from 2 to 8. In certain embodiments, R is present and is a linear or branched, cyclic or straight, saturated or unsaturated alkyl, heteroalkyl, aryl, or heteroaryl chain comprising from 1 to 8 (i.e., 1, 2, 3, 4, 5, 6, 7, or 8) carbon units.

In some embodiments, the compound of Formula VII is selected from:

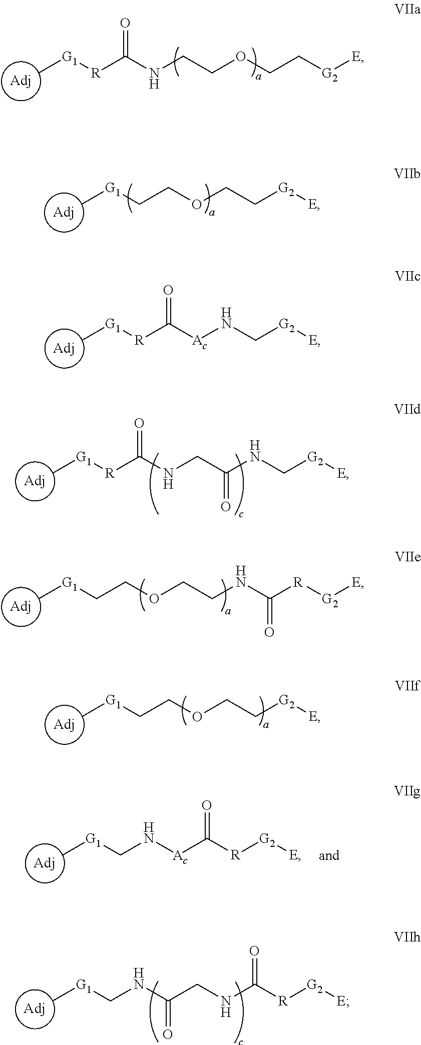

wherein G₁ is CH₂, C═O, or a bond; G₂ is CH₂, C═O, or a bond; R is optionally present and is a linear or branched, cyclic or straight, saturated or unsaturated alkyl, heteroalkyl, aryl, or heteroaryl chain comprising from 1 to 8 carbon units; subscript a is an integer from 1 to 40; each A is independently selected from any amino acid; subscript c is an integer from 1 to 25, and E is an ester. In certain embodiments, subscript a is an integer from 2 to 25. In certain embodiments, subscript c is an integer from 2 to 8.

As previously discussed, there are many ways of forming an immunoconjugate. Each of the prior art methods suffers from downsides. The present method includes a one-step process which conjugates an adjuvant, modified to include a linker, to the lysine side chain of an antibody (compound of Formula VIII). This process is possible by using an ester. The ester can be any suitable ester capable of linking the compound of Formula VII to a lysine side chain of an antibody (compound of Formula VIII).

For example, the ester of Formula VII can be an N-hydroxysuccinimide (“NETS”) ester of the formula:

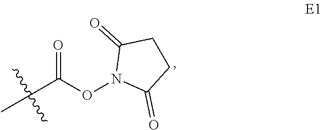 [SMILES: *C(=O)ON1C(=O)CCC1=O]

wherein the wavy line (“ [symbol] ”) represents the point of attachment to G₂.

The ester of Formula VII can also be a sulfo-N-hydroxysuccinimide ester of the formula:

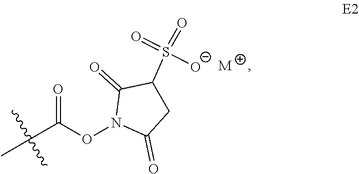

wherein M is any cation and the wavy line (“ [symbol] ”) represents the point of attachment to the G₂. For example, the cation counter ion (“M”) can be a proton, ammonium, a quaternary amine, a cation of an alkali metal, a cation of an alkaline earth metal, a cation of a transition metal, a cation of a rare-earth metal, a main group element cation, or a combination thereof.

The ester of Formula VII can also be a phenol ester of the formula:

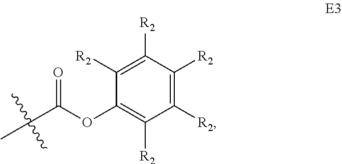

wherein each R₂ is independently selected from hydrogen or fluorine and the wavy line (“ [symbol] ”) represents the point of attachment to G₂.

The ester of Formula VII can also be a phenol ester of the formula:

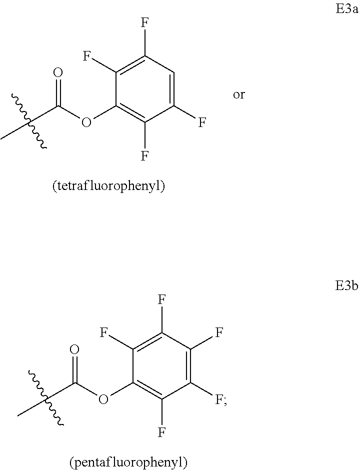 [SMILES: *C(=O)Oc1c(F)c(F)cc(F)c1F ; *C(=O)Oc1c(F)c(F)c(F)c(F)c1F]

wherein the wavy line (“ [symbol] ”) represents the point of attachment to G₂.

In some embodiments, the antibody of Formula VIII and the ester of Formula VII are combined in any suitable aqueous buffer. An exemplary list of suitable aqueous buffers is phosphate buffered saline, borate buffered saline, and tris buffered saline.

Using a tetrafluorophenyl (“TFP”) or pentafluorophenyl (“PFP”) is especially effective in synthesizing the immunoconjugates of the invention.

### Antibodies

The antibodies in the immunoconjugates can be allogeneic antibodies. The terms “allogeneic antibody” or “alloantibody” refer to an antibody that is not from the individual in question (e.g., an individual with a tumor and seeking treatment), but is from the same species, or is from a different species, but has been engineered to reduce, mitigate, or avoid recognition as a xeno-antibody (e.g., non-self). For example, the “allogeneic antibody” can be a humanized antibody. Unless specifically stated otherwise, “antibody” and “allogeneic antibodies” as used herein refer to immunoglobulin G (IgG) or immunoglobulin A (IgA).

If a cancer cell of a human individual is contacted with an antibody that was not generated by that same person (e.g., the antibody was generated by a second human individual, the antibody was generated by another species such as a mouse, the antibody is a humanized antibody that was generated by another species, etc.), then the antibody is considered to be allogeneic (relative to the first individual). A humanized mouse monoclonal antibody that recognizes a human antigen (e.g., a cancer-specific antigen, an antigen that is enriched in and/or on cancer cells, etc.) is considered to be an “alloantibody” (an allogeneic antibody).

In some embodiments, the antibody is a polyclonal allogeneic IgG antibody. In some embodiments, the antibody is present in a mixture of polyclonal IgG antibodies with a plurality of binding specificities. In some cases, the antibodies of the mixture specifically bind to different target molecules, and in some cases the antibodies of the mixture specifically bind to different epitopes of the same target molecule. Thus, a mixture of antibodies can in some cases include more than one immunoconjugate of the invention (e.g., adjuvant moieties can be covalently bonded to antibodies of a mixture, e.g., a mixture of polyclonal IgG antibodies, resulting in a mixture of antibody-adjuvant conjugates of the invention). A mixture of antibodies can be pooled from 2 or more individuals (e.g., 3 or more individuals, 4 or more individuals, 5 or more individuals, 6 or more individuals, 7 or more individuals, 8 or more individuals, 9 or more individuals, 10 or more individuals, etc.). In some cases, pooled serum is used as a source of alloantibody, where the serum can come from any number of individuals, none of whom are the first individual (e.g., the serum can be pooled from 2 or more individuals, 3 or more individuals, 4 or more individuals, 5 or more individuals, 6 or more individuals, 7 or more individuals, 8 or more individuals, 9 or more individuals, 10 or more individuals, etc.). In some cases, the antibodies are isolated or purified from serum prior to use. The purification can be conducted before or after pooling the antibodies from different individuals.

In some cases where the antibodies in the immunoconjugates comprise IgGs from serum, the target antigens for some (e.g., greater than 0% but less than 50%), half, most (greater than 50% but less than 100%), or even all of the antibodies (i.e., IgGs from the serum) will be unknown. However, the chances are high that at least one antibody in the mixture will recognize the target antigen of interest because such a mixture contains a wide variety of antibodies specific for a wide variety of target antigens.

In some embodiments, the antibody is a polyclonal allogeneic IgA antibody. In some embodiments, the antibody is present in a mixture of polyclonal IgA antibodies with a plurality of binding specificities. In some cases, the antibodies of the mixture specifically bind to different target molecules, and in some cases the antibodies of the mixture specifically bind to different epitopes of the same target molecule. Thus, a mixture of antibodies can in some cases include more than one immunoconjugate of the invention (e.g., adjuvant moieties can be covalently bonded to antibodies of a mixture, e.g., a mixture of polyclonal IgA antibodies, resulting in a mixture of antibody-adjuvant conjugates of the invention). A mixture of antibodies can be pooled from 2 or more individuals (e.g., 3 or more individuals, 4 or more individuals, 5 or more individuals, 6 or more individuals, 7 or more individuals, 8 or more individuals, 9 or more individuals, 10 or more individuals, etc.). In some cases, pooled serum is used as a source of alloantibody, where the serum can come from any number of individuals, none of whom are the first individual (e.g., the serum can be pooled from 2 or more individuals, 3 or more individuals, 4 or more individuals, 5 or more individuals, 6 or more individuals, 7 or more individuals, 8 or more individuals, 9 or more individuals, 10 or more individuals, etc.). In some cases, the antibodies are isolated or purified from serum prior to use. The purification can be conducted before or after pooling the antibodies from different individuals.

In some cases where the antibodies in the immunoconjugates comprise IgAs from serum, the target antigens for some (e.g., greater than 0% but less than 50%), half, most (greater than 50% but less than 100%), or even all of the antibodies (i.e., IgAs from the serum) will be unknown. However, the chances are high that at least one antibody in the mixture will recognize the target antigen of interest because such a mixture contains a wide variety of antibodies specific for a wide variety of target antigens.

In some cases, the antibody in the immunoconjugates includes intravenous immunoglobulin (IVIG) and/or antibodies from (e.g., enriched from, purified from, e.g., affinity purified from) IVIG. IVIG is a blood product that contains IgG (immunoglobulin G) pooled from the plasma (e.g., in some cases without any other proteins) from many (e.g., sometimes over 1,000 to 60,000) normal and healthy blood donors. IVIG is commercially available. IVIG contains a high percentage of native human monomeric IVIG, and has low IgA content. When administered intravenously, IVIG ameliorates several disease conditions. Therefore, the United States Food and Drug Administration (FDA) has approved the use of IVIG for a number of diseases including (1) Kawasaki disease; (2) immune-mediated thrombocytopenia; (3) primary immunodeficiencies; (4) hematopoietic stem cell transplantation (for those older than 20 years); (5) chronic B-cell lymphocytic leukemia; and (6) pediatric HIV type 1 infection. In 2004, the FDA approved the Cedars-Sinai IVIG Protocol for kidney transplant recipients so that such recipients could accept a living donor kidney from any healthy donor, regardless of blood type (ABO incompatible) or tissue match. These and other aspects of IVIG are described, for example, in U.S. Patent Application Publications 2010/0150942; 2004/0101909; 2013/0177574; 2013/0108619; and 2013/0011388; which are hereby incorporated by reference in their entirety.

In some cases, the antibody is a monoclonal antibody of a defined sub-class (e.g., IgG₁, IgG₂, IgG₃, IgG₄, IgA₁, or IgA₂). If combinations of antibodies are used, the antibodies can be from the same subclass or from different subclasses. For example, the antibodies can be IgG₁ antibodies. Various combinations of different subclasses, in different relative proportions, can be obtained by those of skill in the art. In some cases, a specific subclass, or a specific combination of different subclasses can be particularly effective at cancer treatment or tumor size reduction. Accordingly, some embodiments of the invention provide immunoconjugates wherein the antibody is a monoclonal antibody. In some embodiments, the monoclonal antibody is humanized.

In some embodiments, the antibody binds to an antigen of a cancer cell. For example, the antibody can bind to a target antigen that is present at an amount of at least 10; 100; 1,000; 10,000; 100,000; 1,000,000; 2.5×10⁶; 5×10⁶; or 1×10⁷ copies or more on the surface of a cancer cell.

In some embodiments, the antibody binds to an antigen on a cancer or immune cell at a higher affinity than a corresponding antigen on a non-cancer cell. For example, the antibody may preferentially recognize an antigen containing a polymorphism that is found on a cancer or immune cell as compared to recognition of a corresponding wild-type antigen on the non-cancer or non-immune cell. In some cases, the antibody binds a cancer or immune cell with greater avidity than a non-cancer or non-immune cell. For example, the cancer or immune cell can express a higher density of an antigen, thus providing for a higher affinity binding of a multivalent antibody to the cancer or immune cell.

In some cases, the antibody does not significantly bind non-cancer antigens (e.g., the antibody binds one or more non-cancer antigens with at least 10; 100; 1,000; 10,000; 100,000; or 1,000,000-fold lower affinity (higher Kd) than the target cancer antigen). In some cases, the target cancer antigen to which the antibody binds is enriched on the cancer cell. For example, the target cancer antigen can be present on the surface of the cancer cell at a level that is at least 2, 5, 10; 100; 1,000; 10,000; 100,000; or 1,000,000-fold higher than a corresponding non-cancer cell. In some cases, the corresponding non-cancer cell is a cell of the same tissue or origin that is not hyperproliferative or otherwise cancerous. In general, a subject IgG antibody that specifically binds to an antigen (a target antigen) of a cancer cell preferentially binds to that particular antigen relative to other available antigens. However, the target antigen need not be specific to the cancer cell or even enriched in cancer cells relative to other cells (e.g., the target antigen can be expressed by other cells). Thus, in the phrase “an antibody that specifically binds to an antigen of a cancer cell,” the term “specifically” refers to the specificity of the antibody and not to the uniqueness of the antigen in that particular cell type.

Modified Fc Region

In some embodiments, the antibodies in the immunoconjugates contain a modified Fc region, wherein the modification modulates the binding of the Fc region to one or more Fc receptors.

The terms “Fc receptor” or “FcR” refer to a receptor that binds to the Fc region of an antibody. There are three main classes of Fc receptors: FcγR which bind to IgG, FcαR which binds to IgA, and FcεR which binds to IgE. The FcγR family includes several members, such as FcγI (CD64), FcγRIIA (CD32A), FcγRIIB (CD32B), FcγRIIIA (CD16A), FcγRIIIB (CD16B). The Fcγ receptors differ in their affinity for IgG and also have different affinities for the IgG subclasses (e.g., IgG₁, IgG₂, IgG₃, IgG₄).

In some embodiments, the antibodies in the immunoconjugates (e.g., antibodies conjugated to a TLR agonist such as a TLR7/8 agonist via a linker) contain one or more modifications (e.g., amino acid insertion, deletion, and/or substitution) in the Fc region that results in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the native antibody lacking the mutation in the Fc region. In some embodiments, the antibodies in the immunoconjugates contain one or more modifications (e.g., amino acid insertion, deletion, and/or substitution) in the Fc region that reduce the binding of the Fc region of the antibody to FcγRIIB In some embodiments, the antibodies in the immunoconjugates contain one or more modifications (e.g., amino acid insertion, deletion, and/or substitution) in the Fc region of the antibody that reduce the binding of the antibody to FcγRIIB while maintaining the same binding or having increased binding to FcγRI (CD64), FcγRIIA (CD32A), and/or FcRγIIIA (CD16a) as compared to the native antibody lacking the mutation in the Fc region. In some embodiments, the antibodies in the immunoconjugates contain one of more modifications in the Fc region that increase the binding of the Fc region of the antibody to FcγRIIB

In some cases, the modulated binding is provided by mutations in the Fc region of the antibody relative to the native Fc region of the antibody. The mutations can be in a CH2 domain, a CH3 domain, or a combination thereof. A “native Fc region” is synonymous with a “wild-type Fc region” and comprises an amino acid sequence that is identical to the amino acid sequence of an Fc region found in nature or identical to the amino acid sequence of the Fc region found in the native antibody (e.g., rituximab). Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc includes the various allotypes of Fcs (see, e.g., Jefferis et al., mAbs, 1(4): 332-338 (2009)).

In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/I332E), GASDALIE (G236A/S239D/A330L/I332E), V9 (G237D/P238D/P271G/A330R), and V11 (G237D/P238D/H268D/P271G/A330R) and/or one or more mutations at the following amino acids: E233, G237, P238, H268, P271, L328 and A330. Additional Fc region modifications for modulating Fc receptor binding are described, e.g., in U.S. Patent Application Publication 2016/0145350, and U.S. Pat. Nos. 7,416,726 and 5,624,821.

In some embodiments, the Fc region of the antibodies of the immunoconjugates are modified to have an altered glycosylation pattern of the Fc region compared to the native non-modified Fc region.

Human immunoglobulin is glycosylated at the Asn297 residue in the Cγ2 domain of each heavy chain. This N-linked oligosaccharide is composed of a core heptasaccharide, N-acetylglucosamine4Mannose3 (GlcNAc4Man3). Removal of the heptasaccharide with endoglycosidase or PNGase F is known to lead to conformational changes in the antibody Fc region, which can significantly reduce antibody-binding affinity to activating FcγR and lead to decreased effector function. The core heptasaccharide is often decorated with galactose, bisecting GlcNAc, fucose or sialic acid, which differentially impacts Fc binding to activating and inhibitory FcγR. Additionally, it has been demonstrated that a2,6-sialyation enhances anti-inflammatory activity in vivo while defucosylation leads to improved FcγRIIIa binding and a 10-fold increase in antibody-dependent cellular cytotoxicity and antibody-dependent phagocytosis. Specific glycosylation patterns can therefore be used to control inflammatory effector functions.

In some embodiments, the modification to alter the glycosylation pattern is a mutation. For example, a substitution at Asn297. In some embodiments, Asn297 is mutated to glutamine (N297Q). Methods for controlling immune response with antibodies that modulate FcγR-regulated signaling are described, for example, in U.S. Pat. No. 7,416,726, as well as US 2007/0014795 and US 2008/0286819.

In some embodiments, the antibodies of the immunoconjugates are modified to contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function. In some embodiments, the antibodies of the immunoconjugates are engineered to be afucosylated (e.g., afucosylated rituximab, available from Invivogen, hcd20-mab13).

In some embodiments, the entire Fc region of an antibody in the immunoconjugates is exchanged with a different Fc region, so that the Fab region of the antibody is conjugated to a non-native Fc region. For example, the Fab region of rituximab, which normally comprises an IgG1 Fc region, can be conjugated to IgG2, IgG3, IgG4, or IgA, or the Fab region of nivolumab, which normally comprises an IgG4 Fc region, can be conjugated to IgG1, IgG2, IgG3, IgA1 or IgG2. In some embodiments, the Fc modified antibody with a non-native Fc domain also comprises one or more amino acid modification, such as the S228P mutation within the IgG4 Fc, that modulate the stability of the Fc domain described. In some embodiments, the Fc modified antibody with a non-native Fc domain also comprises one or more amino acid modifications described herein that modulate Fc binding to FcR.

In some embodiments, the modifications that modulate the binding of the Fc region to FcR do not alter the binding of the Fab region of the antibody to its antigen when compared to the native non-modified antibody. In other embodiments, the modifications that modulate the binding of the Fc region to FcR also increase the binding of the Fab region of the antibody to its antigen when compared to the native non-modified antibody.

Antibody Targets

In some embodiments, the antibody is capable of binding one or more targets selected from (e.g., specifically binds to a target selected from) 5T4, ABL, ABCF1, ACVR1, ACVR1B, ACVR2, ACVR2B, ACVRL1, ADORA2A, Aggrecan, AGR2, AICDA, AIF1, AIGI, AKAP1, AKAP2, AMH, AMHR2, ANGPT1, ANGPT2, ANGPTL3, ANGPTL4, ANPEP, APC, APOC1, AR, aromatase, ATX, AX1, AZGP1 (zinc-a-glycoprotein), B7.1, B7.2, B7-H1, BAD, BAFF, BAG1, BAI1, BCR, BCL2, BCL6, BCMA, BDNF, BLNK, BLR1 (MDR15), BIyS, BMP1, BMP2, BMP3B (GDFIO), BMP4, BMP6, BMP8, BMPR1A, BMPR1B, BMPR2, BPAG1 (plectin), BRCA1, C19orf1O (IL27w), C3, C4A, C5, C5R1, CA9, CANT1, CAPRIN-1, CASP1, CASP4, CAV1, CCBP2 (D6/JAB61), CCL1 (1-309), CCM (eotaxin), CCL13 (MCP-4), CCL15 (MIP-Id), CCL16 (HCC-4), CCL17 (TARC), CCL18 (PARC), CCL19 (MIP-3b), CCL2 (MCP-1), MCAF, CCL20 (MIP-3a), CCL21 (MEP-2), SLC, exodus-2, CCL22(MDC/STC-I), CCL23 (MPIF-I), CCL24 (MPIF-2/eotaxin-2), CCL25 (TECK), CCL26(eotaxin-3), CCL27 (CTACK/ILC), CCL28, CCL3 (MIP-Ia), CCL4 (MIPIb), CCL5(RANTES), CCL7 (MCP-3), CCL8 (mcp-2), CCNA1, CCNA2, CCND1, CCNE1, CCNE2, CCR1 (CKR1/HM145), CCR2 (mcp-IRB/RA), CCR3 (CKR3/CMKBR3), CCR4, CCR5(CMKBR5/ChemR13), CCR6 (CMKBR6/CKR-L3/STRL22/DRY6), CCR7 (CKR7/EBI1), CCR8 or CDw198(CMKBR8/TERI/CKR-L1), CCR9 (GPR-9-6), CCRL1 (VSHK1), CCRL2 (L-CCR), CD164, CD19, CDIC, CD2, CD20, CD21, CD200, CD-22, CD24, CD27, CD28, CD3, CD33, CD35, CD37, CD38, CD3E, CD3G, CD3Z, CD4, CD38, CD40, CD40L, CD44, CD45RB, CD47, CD52, CD69, CD72, CD74, CD79A, CD79B, CD8, CD80, CD81, CD83, CD86, CD137, CD152, CD274, CDH1 (Ecadherin), CDH1O, CDH12, CDH13, CDH18, CDH19, CDH2O, CDH5, CDH7, CDH8, CDH9, CDK2, CDK3, CDK4, CDK5, CDK6, CDK7, CDK9, CDKN1A (p21Wap1/Cip1), CDKN1B (p27Kip1), CDKN1C, CDKN2A (p16INK4a), CDKN2B, CDKN2C, CDKN3, CEBPB, CERT, CHGA, CHGB, Chitinase, CHST1O, CKLFSF2, CKLFSF3, CKLFSF4, CKLFSF5, CKLFSF6, CKLFSF7, CKLFSF8, CLDN3, CLDN7 (claudin-7), CLN3, CLU (clusterin), CMKLR1, CMKOR1 (RDC1), CNR1, COL18A1, COLIA1, COL4A3, COL6A1, CR2, Cripto, CRP, CSF1 (M-CSF), CSF2 (GM-CSF), CSF3 (GCSF), CTAG1B (NY-ESO-1), CTL8, CTNNB1 (b-catenin), CTSB (cathepsin B), CX3CL1 (SCYD1), CX3CR1 (V28), CXCL1 (GRO1), CXCL1O (IP-IO), CXCLI1 (1-TAC/IP-9), CXCL12 (SDF1), CXCL13, CXCL14, CXCL16, CXCL2 (GRO2), CXCL3 (GRO3), CXCL5 (ENA-78/LIX), CXCL6 (GCP-2), CXCL9 (MIG), CXCR3 (GPR9/CKR-L2), CXCR4, CXCR6 (TYMSTR/STRL33/Bonzo), CYB5, CYC1, CYSLTR1, DAB2IP, DES, DKFZp451J0118, DNCL1, DPP4, E2F1, Engel, Edge, Fennel, EFNA3, EFNB2, EGF, EGFR, ELAC2, ENG, Enola, ENO2, ENO3, EPHA1, EPHA2, EPHA3, EPHA4, EPHA5, EPHA6, EPHA7, EPHA8, EPHA9, EPHA10, EPHB1, EPHB2, EPHB3, EPHB4, EPHB5, EPHB6, EPHRIN-A1, EPHRIN-A2, EPHRINA3, EPHRIN-A4, EPHRIN-A5, EPHRIN-A6, EPHRIN-B1, EPHRIN-B2, EPHRIN-B3, EPHB4, EPG, ERBB2 (HER2), EREG, ERK8, Estrogen receptor, Earl, ESR2, F3 (TF), FADD, farnesyltransferase, FasL, FASNf, FCER1A, FCER2, FCGR3A, FGF, FGF1 (aFGF), FGF10, FGF11, FGF12, FGF12B, FGF13, FGF14, FGF16, FGF17, FGF18, FGF19, FGF2 (bFGF), FGF20, FGF21, FGF22, FGF23, FGF3 (int-2), FGF4 (HST), FGF5, FGF6 (HST-2), FGF7 (KGF), FGF8, FGF9, FGFR3, FIGF (VEGFD), FIL1(EPSILON), FBL1 (ZETA), FLJ12584, FLJ25530, FLRT1 (fibronectin), FLT1, FLT-3, FOLR1, FOS, FOSL1(FRA-1), FY (DARC), GABRP (GABAa), GAGEB1, GAGEC1, GALNAC4S-6ST, GATA3, GD2, GDF5, GFI1, GGT1, GM-CSF, GNAS1, GNRH1, GPC3, GPR2 (CCR10), GPR31, GPR44, GPR81 (FKSG80), GRCC1O (C1O), GRP, GSN (Gelsolin), GSTP1, HAVCR2, HDAC, HDAC4, HDAC5, HDAC7A, HDAC9, Hedgehog, HGF, HIF1A, HIP1, histamine and histamine receptors, HLA-A, HLA-DRA, HLA-E, HM74, HMOXI, HSP90, HUMCYT2A, ICEBERG, ICOSL, ID2, IFN-a, IFNA1, IFNA2, IFNA4, IFNA5, EFNA6, BFNA7, IFNB1, IFNgamma, IFNW1, IGBP1, IGF1, IGFIR, IGF2, IGFBP2, IGFBP3, IGFBP6, DL-1, ILIO, ILIORA, ILIORB, IL-1, IL1R1 (CD121a), IL1R2(CD121b), IL-IRA, IL-2, IL2RA (CD25), IL2RB(CD122), IL2RG(CD132), IL-4, IL-4R(CD123), IL-5, IL5RA(CD125), IL3RB(CD131), IL-6, IL6RA, (CD126), IR6RB(CD13O), IL-7, IL7RA(CD127), IL-8, CXCR1 (IL8RA), CXCR2, (IL8RB/CD128), IL-9, IL9R(CD129), IL-10, IL10RA(CD210), IL10RB(CDW210B), IL-11, IL11RA, IL-12, IL-12A, IL-12B, IL-12RB1, IL-12RB2, IL-13, IL13RA1, IL13RA2, IL14, IL15, IL15RA, IL16, IL17, IL17A, IL17B, IL17C, IL17R, IL18, IL18BP, IL18R1, IL18RAP, IL19, ILIA, ILIB, ILIF10, ILIF5, IL1F6, ILIF7, IL1F8, DL1F9, ILIHYI, ILIR1, IL1R2, ILIRAP, ILIRAPLI, ILIRAPL2, ILIRL1, IL1RL2, ILIRN, IL2, IL20, IL20RA, IL21R, IL22, IL22R, IL22RA2, IL23, DL24, IL25, IL26, IL27, IL28A, IL28B, IL29, IL2RA, IL2RB, IL2RG, IL3, IL30, IL3RA, IL4, 1L4, IL6ST (glycoprotein 130), ILK, INHA, INHBA, INSL3, INSL4, IRAK1, IRAK2, ITGA1, ITGA2, ITGA3, ITGA6 (α6 integrin), ITGAV, ITGB3, ITGB4 (β34 integrin), JAG1, JAK1, JAK3, JTB, JUN, K6HF, KAI1, KDR, KITLG, KLFS (GC Box BP), KLF6, KLK10, KLK12, KLK13, KLK14, KLK15, KLK3, KLK4, KLKS, KLK6, KLK9, KRT1, KRT19 (Keratin 19), KRT2A, KRTHB6(hair-specific type II keratin), L1CAM, LAG3, LAMA5, LEP (leptin), Lewis Y antigen, Lingo-p75, Lingo-Troy, LRRC15, LPS, LTA (TNF-b), LTB, LTB4R (GPR16), LTB4R2, LTBR, MACMARCKS, MAG or OMgp, MAGEA3, MAGEA6, MAP2K7 (c-Jun), MCP-1, MDK, MIB1, midkine, MIF, MISRII, MJP-2, MSLN, MK, MKI67 (Ki-67), MMP2, MMP9, MS4A1, MSMB, MT3 (metallothionectin-UI), mTOR, MTSS1, MUC1 (mucin), MYC, MYD88, NCK2, neurocan, NFKBI, NFKB2, NGFB (NGF), NGFR, NgR-Lingo, NgRNogo66, (Nogo), NgR-p75, NgR-Troy, NMEI (NM23A), NOTCH, NOTCH1, NOX5, NPPB, NROB1, NROB2, NRID1, NR1D2, NR1H2, NR1H3, NR1H4, NR112, NR113, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRP1, NRP2, NT5E, NTN4, ODZI, OPRDI, P2RX7, PAP, PART1, PATE, PAWR, PCA3, PCDGF, PCNA, PDGFA, PDGFB, PDGFRA, PDGFRB, PECAMI, peg-asparaginase, PF4 (CXCL4), PGF, PGR, phosphacan, PIAS2, PI3 Kinase, PIK3CG, PLAU (uPA), PLG, PLXDCI, PKC, PKC-beta, PPBP (CXCL7), PPID, PR1, PRKCQ, PRKD1, PRL, PROC, PROK2, PSAP, PSCA, PSMA, PTAFR, PTEN, PTGS2 (COX-2), PTN, PVRIG, RAC2 (P21Rac2), RANK, RANK ligand, RARB, RGS1, RGS13, RGS3, RNFI1O (ZNF144), Ron, ROBO2, ROR1, RXR, S100A2, SCGB 1D2 (lipophilin B), SCGB2A1 (mammaglobin 2), SCGB2A2 (mammaglobin 1), SCYE1 (endothelial Monocyte-activating cytokine), SDF2, SERPENA1, SERPINA3, SERPINB5 (maspin), SERPINEI (PAM), SERPINFI, SHIP-1, SHIP-2, SHB1, SHB2, SHBG, SfcAZ, SLC2A2, SLC33A1, SLC43A1, SLIT2, SPP1, SPRR1B (Spr1), ST6GAL1, STAB1, STATE, STEAP, STEAP2, TB4R2, TBX21, TCP1O, TDGF1, TEK, TGFA, TGFB1, TGFB1I1, TGFB2, TGFB3, TGFBI, TGFBR1, TGFBR2, TGFBR3, THIL, THBS1 (thrombospondin-1), THBS2, THBS4, THPO, TIE (Tie-1), TIMP3, tissue factor, TLR1, TLR2, TLR3, TLR4, TLR5, TLR6, TLR7, TLR8, TLR9, TLR10, TLR11, TNF, TNF-a, TNFAIP2 (B94), TNFAIP3, TNFRSFI1A, TNFRSF1A, TNFRSF1B, TNFRSF21, TNFRSF5, TNFRSF6 (Fas), TNFRSF7, TNFRSF8, TNFRSF9, TNFSF1O (TRAIL), TNFSF11 (TRANCE), TNFSF12 (APO3L), TNFSF13 (April), TNFSF13B, TNFSF14 (HVEM-L), TNFRSF14 (HVEM), TNFSF15 (VEGI), TNFSF18, TNFSF4 (OX40 ligand), TNFSF5 (CD40 ligand), TNFSF6 (FasL), TNFSF7 (CD27 ligand), TNFSF8 (CD30 ligand), TNFSF9 (4-1BB ligand), TOLLIP, Toll-like receptors, TOP2A (topoisomerase Iia), TP53, TPM1, TPM2, TRADD, TRAF1, TRAF2, TRAF3, TRAF4, TRAF5, TRAF6, TRKA, TREM1, TREM2, TROP2, TRPC6, TSLP, TWEAK, Tyrosinase, uPAR, VEGF, VEGFB, VEGFC, versican, VHL C5, VLA-4, WT1, Wnt-1, XCL1 (lymphotactin), XCL2 (SCM-Ib), XCRI (GPR5/CCXCR1), YY1, ZFPM2, CLEC4C (BDCA-2, DLEC, CD303, CLECSF7), CLEC4D (MCL, CLECSF8), CLEC4E (Mincle), CLEC6A (Dectin-2), CLEC5A (MDL-1, CLECSF5), CLEC1B (CLEC-2), CLEC9A (DNGR-1), CLEC7A (Dectin-1), CLEC11A, PDGFRa, SLAMF7, GP6 (GPVI), LILRA1 (CD85I), LILRA2 (CD85H, ILT1), LILRA4 (CD85G, ILT7), LILRA5 (CD85F, ILT11), LILRA6 (CD85b, ILT8), LILRB1, NCR1 (CD335, LY94, NKp46), NCR3 (CD335, LY94, NKp46), NCR3 (CD337, NKp30), OSCAR, TARM1, CD300C, CD300E, CD300LB (CD300B), CD300LD (CD300D), KIR2DL4 (CD158D), KIR2DS, KLRC2 (CD159C, NKG2C), KLRK1 (CD314, NKG2D), NCR2 (CD336, NKp44), PILRB, SIGLEC1 (CD169, SN), SIGLEC5, SIGLEC6, SIGLEC7, SIGLEC8, SIGLEC9, SIGLEC10, SIGLEC11, SIGLEC12, SIGLEC14, SIGLEC15 (CD33L3), SIGLEC16, SIRPA, SIRPB1 (CD172B), TREM1 (CD354), TREM2, and KLRF1 (NKp80).

In some embodiments, the antibody binds to an FcRγ-coupled receptor. In some embodiments, the FcRγ-coupled receptor is selected from the group consisting of GP6 (GPVI), LILRA1 (CD85I), LILRA2 (CD85H, ILT1), LILRA4 (CD85G, ILT7), LILRA5 (CD85F, ILT11), LILRA6 (CD85b, ILT8), LILRB1, NCR1 (CD335, LY94, NKp46), NCR3 (CD335, LY94, NKp46), NCR3 (CD337, NKp30), OSCAR, and TARM1.

In some embodiments, the antibody binds to a DAP12-coupled receptor. In some embodiments, the DAP12-coupled receptor is selected from the group consisting of CD300C, CD300E, CD300LB (CD300B), CD300LD (CD300D), KIR2DL4 (CD158D), KIR2DS, KLRC2 (CD159C, NKG2C), KLRK1 (CD314, NKG2D), NCR2 (CD336, NKp44), PILRB, SIGLEC1 (CD169, SN), SIGLEC5, SIGLEC6, SIGLEC7, SIGLEC8, SIGLEC9, SIGLEC10, SIGLEC11, SIGLEC12, SIGLEC14, SIGLEC15 (CD33L3), SIGLEC16, SIRPB1 (CD172B), TREM1 (CD354), and TREM2.

In some embodiments, the antibody binds to a hemITAM-bearing receptor. In some embodiments, the hemITAM-bearing receptor is KLRF1 (NKp80).

In some embodiments, the antibody is capable of binding one or more targets selected from CLEC4C (BDCA-2, DLEC, CD303, CLECSF7), CLEC4D (MCL, CLECSF8), CLEC4E (Mincle), CLEC6A (Dectin-2), CLEC5A (MDL-1, CLECSF5), CLEC1B (CLEC-2), CLEC9A (DNGR-1), and CLEC7A (Dectin-1). In some embodiments, the antibody is capable of binding CLEC6A (Dectin-2) or CLEC5A. In some embodiments, the antibody is capable of binding CLEC6A (Dectin-2).

In some embodiments, the antibody is capable of binding one or more targets selected from (e.g., specifically binds to a target selected from): ATP5I (Q06185), OAT (P29758), AIFM1 (Q9Z0X1), AOFA (Q64133), MTDC (P18155), CMC1 (Q8BH59), PREP (Q8K411), YMEL1 (088967), LPPRC (Q6PB66), LONM (Q8CGK3), ACON (Q99KI0), ODO1 (Q60597), IDHP (P54071), ALDH2 (P47738), ATPB (P56480), AATM (P05202), TM:1V193 (Q9CQW0), ERGI3 (Q9CQE7), RTN4 (Q99P72), CL041 (Q8BQR4), ERLN2 (Q8BFZ9), TERA (Q01853), DAD1 (P61804), CALX (P35564), CALU (035887), VAPA (Q9WV55), MOGS (Q80UM7), GANAB (Q8BHN3), ERO1A (Q8R180), UGGG1 (Q6P5E4), P4HA1 (Q60715), HYEP (Q9D379), CALR (P14211), AT2A2 (055143), PDIA4 (P08003), PDIA1 (P09103), PDIA3 (P27773), PDIA6 (Q922R8), CLH (Q68FD5), PPIB (P24369), TCPG (P80318), MOT4 (P57787), NICA (P57716), BASI (P18572), VAPA (Q9WV55), ENV2 (P11370), VAT1 (Q62465), 4F2 (P10852), ENOA (P17182), ILK (055222), GPNMB (Q99P91), ENV1 (P10404), ERO1A (Q8R180), CLH (Q68FD5), DSG1A (Q61495), AT1A1 (Q8VDN2), HYOU1 (Q9JKR6), TRAP1 (Q9CQN1), GRP75 (P38647), ENPL (P08113), CH60 (P63038), and CH10 (Q64433). In the preceding list, accession numbers are shown in parentheses.

In some embodiments, the antibody binds to an antigen selected from CCR8, CDH1, CD19, CD20, CD29, CD30, CD38, CD40, CD47, EpCAM, MUC1, MUC16, EGFR, HER2, SLAMF7, and gp75. In some embodiments, the antigen is selected from CCR8, CD19, CD20, CD47, EpCAM, MUC1, MUC16, EGFR, and HER2. In some embodiments, the antibody binds to an antigen selected from the Tn antigen and the Thomsen-Friedenreich antigen. In some embodiments, the antibody binds to an antigen selected from EGFR, CCR8, and HER2. In certain embodiments, the antibody binds to HER2.

In some embodiments, the antibody or Fc fusion protein is selected from: abagovomab, abatacept (also known as ORENCIA™), abciximab (also known as REOPRO™, c7E3 Fab), adalimumab (also known as HUMIRA™), adecatumumab, alemtuzumab (also known as CAMPATH™, MabCampath or Campath-1H), altumomab, afelimomab, anatumomab mafenatox, anetumumab, anrukizumab, apolizumab, arcitumomab, aselizumab, atlizumab, atorolimumab, bapineuzumab, basiliximab (also known as SIMULECT™), bavituximab, bectumomab (also known as LYMPHOSCAN™), belimumab (also known as LYMPHO-STAT-B™), bertilimumab, besilesomab, bevacizumab (also known as AVASTIN™), biciromab brallobarbital, bivatuzumab mertansine, campath, canakinumab (also known as ACZ885), cantuzumab mertansine, capromab (also known as PROSTASCINT™), catumaxomab (also known as REMOVAB™), cedelizumab (also known as CIMZIA™), certolizumab pegol, cetuximab (also known as ERBITUX™), clenoliximab, dacetuzumab, dacliximab, daclizumab (also known as ZENAPAX™), denosumab (also known as AMG 162), detumomab, dorlimomab aritox, dorlixizumab, duntumumab, durimulumab, durmulumab, ecromeximab, eculizumab (also known as SOLIRIS™), edobacomab, edrecolomab (also known as Mab17-1A, PANOREX™), efalizumab (also known as RAPTIVA™), efungumab (also known as MYCOGRAB™) elsilimomab, enlimomab pegol, epitumomab cituxetan, efalizumab, epitumomab, epratuzumab, erlizumab, ertumaxomab (also known as REXOMUN™), etanercept (also known as ENBREL™), etaracizumab (also known as etaratuzumab, VITAXIN™, ABEGRIN™) exbivirumab, fanolesomab (also known as NEUTROSPEC™), faralimomab, felvizumab, fontolizumab (also known as HUZAF™), galiximab, gantenerumab, gavilimomab (also known as ABXCBL™), gemtuzumab ozogamicin (also known as MYLOTARG™), golimumab (also known as CNTO 148), gomiliximab, ibalizumab (also known as TNX-355), ibritumomab tiuxetan (also known as ZEVALIN™), igovomab, imciromab, infliximab (also known as REMICADE™), inolimomab, inotuzumab ozogamicin, ipilimumab (also known as MDX-010, MDX-101), iratumumab, keliximab, labetuzumab, lemalesomab, lebrilizumab, lerdelimumab, lexatumumab (also known as, HGS-ETR2, ETR2-ST01), lexitumumab, libivirumab, lintuzumab, lucatumumab, lumiliximab, mapatumumab (also known as HGSETR1, TRM-1), maslimomab, matuzumab (also known as EMD72000), mepolizumab (also known as BOSATRIA™), metelimumab, milatuzumab, minretumomab, mitumomab, morolimumab, motavizumab (also known as NUMAX™), muromonab (also known as OKT3), nacolomab tafenatox, naptumomab estafenatox, natalizumab (also known as TYSABRI™, ANTEGREN™), nebacumab, nerelimomab, nimotuzumab (also known as THERACIM hR3™, THERA-CIM-hR3™, THERALOC™), nofetumomab merpentan (also known as VERLUMA™), ocrelizumab, odulimomab, ofatumumab, omalizumab (also known as XOLAIR™), oregovomab (also known as OVAREX™), otelixizumab, pagibaximab, palivizumab (also known as SYNAGIS™), panitumumab (also known as ABX-EGF, VECTIBIX™), pascolizumab, pemtumomab (also known as THERAGYN™), pertuzumab (also known as 2C4, OMNITARG™), pexelizumab, pintumomab, priliximab, pritumumab, ranibizumab (also known as LUCENTIS™), raxibacumab, regavirumab, reslizumab, rituximab (also known as RITUXAN™, MabTHERA™), rovelizumab, ruplizumab, satumomab, sevirumab, sibrotuzumab, siplizumab (also known as MEDI-507), sontuzumab, stamulumab (also known as MYO-029), sulesomab (also known as LEUKOSCAN™), tacatuzumab tetraxetan, tadocizumab, talizumab, taplitumomab paptox, tefibazumab (also known as AUREXIS™), telimomab aritox, teneliximab, teplizumab, ticilimumab, tocilizumab (also known as ACTEMRA™), toralizumab, tositumomab, trastuzumab (also known as HERCEPTIN™), tremelimumab (also known as CP-675,206), tucotuzumab celmoleukin, tuvirumab, urtoxazumab, ustekinumab (also known as CNTO 1275), vapaliximab, veltuzumab, vepalimomab, visilizumab (also known as NUVION™), volociximab (also known as M200), votumumab (also known as HUMASPECT™), zalutumumab, zanolimumab (also known as HuMAX-CD4), ziralimumab, zolimomab aritox, daratumumab, elotuxumab, obintunzumab, olaratumab, brentuximab vedotin, afibercept, abatacept, belatacept, afibercept, etanercept, romiplostim, SBT-040 (sequences listed in US 2017/0158772. In some embodiments, the antibody is trastuzumab, cetuximab, panitumumab, zalutumumab, nimotuzumab, or matuzumab. In certain embodiments, the antibody is trastuzumab.

Checkpoint Inhibitors

Any suitable immune checkpoint inhibitor is contemplated for use with the immunoconjugates disclosed herein. In some embodiments, the immune checkpoint inhibitor reduces the expression or activity of one or more immune checkpoint proteins. In another embodiment, the immune checkpoint inhibitor reduces the interaction between one or more immune checkpoint proteins and their ligands. Inhibitory nucleic acids that decrease the expression and/or activity of immune checkpoint molecules can also be used in the methods disclosed herein.

Most checkpoint antibodies are designed not to have effector function as they are not trying to kill cells, but rather to block the signaling. Immunoconjugates of the invention can add back the “effector functionality” needed to activate myeloid immunity. Hence, for most checkpoint antibody inhibitors this discovery will be critical.

In some embodiments, the immune checkpoint inhibitor is cytotoxic T-lymphocyte antigen 4 (CTLA4, also known as CD152), T cell immunoreceptor with Ig and ITIM domains (TIGIT), glucocorticoid-induced TNFR-related protein (GITR, also known as TNFRSF18), inducible T cell costimulatory (ICOS, also known as CD278), CD96, poliovirus receptor-related 2 (PVRL2, also known as CD112R, programmed cell death protein 1 (PD-1, also known as CD279), programmed cell death 1 ligand 1 (PD-L1, also known as B7-H3 and CD274), programmed cell death ligand 2 (PD-L2, also known as B7-DC and CD273), lymphocyte activation gene-3 (LAG-3, also known as CD223), B7-H4, killer immunoglobulin receptor (KIR), Tumor Necrosis Factor Receptor superfamily member 4 (TNFRSF4, also known as OX40 and CD134) and its ligand OX4OL (CD252), indoleamine 2,3-dioxygenase 1 (IDO-1), indoleamine 2,3-dioxygenase 2 (IDO-2), carcinoembryonic antigen-related cell adhesion molecule 1 (CEACAM1), B and T lymphocyte attenuator (BTLA, also known as CD272), T-cell membrane protein 3 (TIM3), the adenosine A2A receptor (A2Ar), and V-domain Ig suppressor of T cell activation (VISTA protein). In some embodiments, the immune checkpoint inhibitor is an inhibitor of CTLA4, PD-1, or PD-L1.

In some embodiments, the antibody is selected from: ipilimumab (also known as Yervoy®) pembrolizumab (also known as Keytruda®), nivolumab (also known as Opdivo®), atezolizumab (also known as Tecentrig®), avelumab (also known as Bavencio®), and durvalumab (also known as Imfinzi™). In some embodiments, the antibody is selected from: ipilimumab (also known as Yervoy®), pembrolizumab (also known as Keytruda®), nivolumab (also known as Opdivo®), and atezolizumab (also known as Tecentrig®).

In some embodiments, the immune checkpoint inhibitor is an inhibitor of CTLA4. In some embodiments, the immune checkpoint inhibitor is an antibody against CTLA4. In some embodiments, the immune checkpoint inhibitor is a monoclonal antibody against CTLA4. In some embodiments, the immune checkpoint inhibitor is a human or humanized antibody against CTLA4. In some embodiments, the immune checkpoint inhibitor reduces the expression or activity of one or more immune checkpoint proteins, such as CTLA4.

In some embodiments, the immune checkpoint inhibitor is an inhibitor of PD-1. In some embodiments, the immune checkpoint inhibitor is an antibody against PD-1. In some embodiments, the immune checkpoint inhibitor is a monoclonal antibody against PD-1. In some embodiments, the immune checkpoint inhibitor is a human or humanized antibody against PD-1. In some embodiments, the immune checkpoint inhibitor reduces the expression or activity of one or more immune checkpoint proteins, such as PD-1.

In some embodiments, the immune checkpoint inhibitor is an inhibitor of PD-L1. In some embodiments, the immune checkpoint inhibitor is an antibody against PD-L1. In some embodiments, the immune checkpoint inhibitor is a monoclonal antibody against PD-L1. In some embodiments, the immune checkpoint inhibitor is a human or humanized antibody against PD-L1. In some embodiments, the immune checkpoint inhibitor reduces the expression or activity of one or more immune checkpoint proteins, such as PD-L1. In some embodiments, the immune checkpoint inhibitor reduces the interaction between PD-1 and PD-L1.

In some embodiments, the immune checkpoint inhibitor is an inhibitor of PD-L2. In some embodiments, the immune checkpoint inhibitor is an antibody against PD-L2. In some embodiments, the immune checkpoint inhibitor is a monoclonal antibody against PD-L2. In some embodiments, the immune checkpoint inhibitor is a human or humanized antibody against PD-L2. In some embodiments, the immune checkpoint inhibitor reduces the expression or activity of one or more immune checkpoint proteins, such as PD-L2. In some embodiments, the immune checkpoint inhibitor reduces the interaction between PD-1 and PD-L2.

In some embodiments, the immune checkpoint inhibitor is an inhibitor of LAG-3. In some embodiments, the immune checkpoint inhibitor is an antibody against LAG-3. In some embodiments, the immune checkpoint inhibitor is a monoclonal antibody against LAG-3. In some embodiments, the immune checkpoint inhibitor is a human or humanized antibody against LAG-3. In some embodiments, the immune checkpoint inhibitor reduces the expression or activity of one or more immune checkpoint proteins, such as LAG-3.

In some embodiments, the immune checkpoint inhibitor is an inhibitor of B7-H4. In some embodiments, the immune checkpoint inhibitor is an antibody against B7-H4. In some embodiments, the immune checkpoint inhibitor is a monoclonal antibody against B7-H4. In some embodiments, the immune checkpoint inhibitor is a human or humanized antibody against B7-H4. In some embodiments, the immune checkpoint inhibitor reduces the expression or activity of one or more immune checkpoint proteins, such as B7-H4.

In some embodiments, the immune checkpoint inhibitor is an inhibitor of KIR. In some embodiments, the immune checkpoint inhibitor is an antibody against KIR. In some embodiments, the immune checkpoint inhibitor is a monoclonal antibody against KIR. In some embodiments, the immune checkpoint inhibitor is a human or humanized antibody against KIR. In some embodiments, the immune checkpoint inhibitor reduces the expression or activity of one or more immune checkpoint proteins, such as KIR.

In some embodiments, the immune checkpoint inhibitor is an inhibitor of TNFRSF4. In some embodiments, the immune checkpoint inhibitor is an antibody against TNFRSF4. In some embodiments, the immune checkpoint inhibitor is a monoclonal antibody against TNFRSF4. In some embodiments, the immune checkpoint inhibitor is a human or humanized antibody against TNFRSF4. In some embodiments, the immune checkpoint inhibitor reduces the expression or activity of one or more immune checkpoint proteins, such as TNFRSF4.

In some embodiments, the immune checkpoint inhibitor is an inhibitor of OX40L. In some embodiments, the immune checkpoint inhibitor is an antibody against OX40L. In some embodiments, the immune checkpoint inhibitor is a monoclonal antibody against OX40L. In some embodiments, the immune checkpoint inhibitor is a human or humanized antibody against OX40L. In some embodiments, the immune checkpoint inhibitor reduces the expression or activity of one or more immune checkpoint proteins, such as OX40L. In some embodiments, the immune checkpoint inhibitor reduces the interaction between TNFRSF4 and OX40L.

In some embodiments, the immune checkpoint inhibitor is an inhibitor of IDO-1. In some embodiments, the immune checkpoint inhibitor is an antibody against IDO-1. In some embodiments, the immune checkpoint inhibitor is a monoclonal antibody against IDO-1. In some embodiments, the immune checkpoint inhibitor is a human or humanized antibody against IDO-1. In some embodiments, the immune checkpoint inhibitor reduces the expression or activity of one or more immune checkpoint proteins, such as IDO-1.

In some embodiments, the immune checkpoint inhibitor is an inhibitor of IDO-2. In some embodiments, the immune checkpoint inhibitor is an antibody against IDO-2. In some embodiments, the immune checkpoint inhibitor is a monoclonal antibody against IDO-2. In some embodiments, the immune checkpoint inhibitor is a human or humanized antibody against IDO-2. In some embodiments, the immune checkpoint inhibitor reduces the expression or activity of one or more immune checkpoint proteins, such as IDO-2.

In some embodiments, the immune checkpoint inhibitor is an inhibitor of CEACAM1. In some embodiments, the immune checkpoint inhibitor is an antibody against CEACAM1. In some embodiments, the immune checkpoint inhibitor is a monoclonal antibody against CEACAM1. In some embodiments, the immune checkpoint inhibitor is a human or humanized antibody against CEACAM1. In some embodiments, the immune checkpoint inhibitor reduces the expression or activity of one or more immune checkpoint proteins, such as CEACAM1.

In some embodiments, the immune checkpoint inhibitor is an inhibitor of BTLA. In some embodiments, the immune checkpoint inhibitor is an antibody against BTLA. In some embodiments, the immune checkpoint inhibitor is a monoclonal antibody against BTLA. In some embodiments, the immune checkpoint inhibitor is a human or humanized antibody against BTLA. In some embodiments, the immune checkpoint inhibitor reduces the expression or activity of one or more immune checkpoint proteins, such as BTLA.

In some embodiments, the immune checkpoint inhibitor is an inhibitor of TIM3. In some embodiments, the immune checkpoint inhibitor is an antibody against TIM3. In some embodiments, the immune checkpoint inhibitor is a monoclonal antibody against TIM3. In some embodiments, the immune checkpoint inhibitor is a human or humanized antibody against TIM3. In some embodiments, the immune checkpoint inhibitor reduces the expression or activity of one or more immune checkpoint proteins, such as TIM3.

In some embodiments, the immune checkpoint inhibitor is an inhibitor of A2Ar. In some embodiments, the immune checkpoint inhibitor is an antibody against A2Ar. In some embodiments, the immune checkpoint inhibitor is a monoclonal antibody against A2Ar. In some embodiments, the immune checkpoint inhibitor is a human or humanized antibody against A2Ar. In some embodiments, the immune checkpoint inhibitor reduces the expression or activity of one or more immune checkpoint proteins, such as A2Ar.

In some embodiments, the immune checkpoint inhibitor is an inhibitor of VISTA protein. In some embodiments, the immune checkpoint inhibitor is an antibody against VISTA protein. In some embodiments, the immune checkpoint inhibitor is a monoclonal antibody against VISTA protein. In some embodiments, the immune checkpoint inhibitor is a human or humanized antibody against VISTA protein. In some embodiments, the immune checkpoint inhibitor reduces the expression or activity of one or more immune checkpoint proteins, such as VISTA protein.

Biosimilars

The immunoconjugates of the invention are likely effective with antibody constructs that are highly similar, or biosimilar, to the commercially available, or “innovator”, antibody constructs. Biosimilar immunoconjugates will likely elicit myeloid activation as effectively as the commercially available antibodies.

DAR Ratios

The immunoconjugates of the invention provide DAR ratios which are desirable. For example, a DAR ratio of about 1.

Isotype Modification

When the IgG1 Fc region of an antibody, such as rituximab, is exchanged for IgG1 AF, IgG1 NQ, IgG2, IgG3, IgG4, or IgA2, and then formed into an immunoconjugates of the invention, the activity of the immunoconjugate can be modulated and often, improved, for the desired application.

Around 30% of human IgG is glycosylated within the Fab region, and the antibody in the immunoconjugates of the invention can contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function.

Antibodies for forming immunoconjugates can contain engineered (i.e., non-naturally occurring) cysteine residues characterized by altered (e.g., enhanced) reactivity toward the reagents used for covalently bonding the adjuvant moieties to the antibodies. In certain embodiments, an engineered cysteine residue will have a thiol reactivity value in the range of 0.6 to 1.0. In many cases, the engineered antibody will be more reactive than the parent antibody.

In general, the engineered residues are “free” cysteine residues that are not part of disulfide bridges. The term “thiol reactivity value” is a quantitative characterization of the reactivity of free cysteine amino acids. As used herein, the term “thiol reactivity value” refers to the percentage of a free cysteine amino acid in an engineered antibody which reacts with a thiol-reactive reagent, and converted to a maximum value of 1. For example, a cysteine residue in an engineered antibody which reacts in 100% yield with a thiol-reactive reagent, such as a maleimide, to form a modified antibody has a thiol reactivity value of 1.0. Another cysteine residue engineered into the same or different parent antibody which reacts in 80% yield with a thiol-reactive reagent has a thiol reactivity value of 0.8. Determination of the thiol reactivity value of a particular cysteine residue can be conducted by ELISA assay, mass spectroscopy, liquid chromatography, autoradiography, or other quantitative analytical tests.

Engineered cysteine residues can be located in the antibody heavy chains or the antibody light chains. In certain embodiments, engineered cysteine residues are located in the Fc region of the heavy chains. For example, amino acid residues at positions L-15, L-43, L-110, L-144, and L-168 in the light chains of an antibody or H-40, H-88, H-119, H-121, H-122, H-175, and H-179 in the heavy chains of an antibody can be replaced with cysteine residues. Positions within about 5 amino acid residues on each side of these positions can also be replaced with cysteine residues, i.e., L-10 to L-20; L-38 to L-48; L-105 to L-115; L-139 to L-149; L-163 to L-173; H-35 to H-45; H-83 to H-93; H-114 to H-127; and H-170 to H-184, as well as the positions in the Fc region selected from H-268 to H-291; H-319 to H-344; H-370 to H-380; and H-395 to H-405, to provide useful cysteine engineered antibodies for forming immunoconjugates. Other engineered antibodies are described, for example, in U.S. Pat. Nos. 7,855,275; 8,309,300; and 9,000,130, which are hereby incorporated by reference.

In addition to antibodies, alternative protein scaffolds may be used as part of the immunoconjugates. The term “alternative protein scaffold” refers to a non-immunoglobulin derived protein or peptide. Such proteins and peptides are generally amenable to engineering and can be designed to confer monospecificity against a given antigen, bispecificity, or multispecificity. Engineering of an alternative protein scaffold can be conducted using several approaches. A loop grafting approach can be used where sequences of known specificity are grafted onto a variable loop of a scaffold. Sequence randomization and mutagenesis can be used to develop a library of mutants, which can be screened using various display platforms (e.g., phage display) to identify a novel binder. Site-specific mutagenesis can also be used as part of a similar approach. Alternative protein scaffolds exist in a variety of sizes, ranging from small peptides with minimal secondary structure to large proteins of similar size to a full-sized antibody. Examples of scaffolds include, but are not limited to, cystine knotted miniproteins (also known as knottins), cyclic cystine knotted miniproteins (also known as cyclotides), avimers, affibodies, the tenth type III domain of human fibronectin, DARPins (designed ankyrin repeats), and anticalins (also known as lipocalins). Naturally occurring ligands with known specificity can also be engineered to confer novel specificity against a given target. Examples of naturally occurring ligands that may be engineered include the EGF ligand and VEGF ligand. Engineered proteins can either be produced as monomeric proteins or as multimers, depending on the desired binding strategy and specificities. Protein engineering strategies can be used to fuse alternative protein scaffolds to Fc domains.

### Preparation of Antibody Adjuvant Conjugates

Reactions for forming the immunoconjugates of the invention are conducted under conditions sufficient to covalently bond the adjuvant moiety to the antibody. In general, the reactions are conducted by contacting an antibody with an adjuvant-linker compound such that an amino acid sidechain in the antibody reacts with the adjuvant linker compound. In some embodiments, the adjuvant-linker compound and the antibody are used in approximately equimolar amounts when forming the immunoconjugates. In some embodiments, an excess of the adjuvant-linker compound is used when forming the immunoconjugates. For example, a reaction mixture for forming an immunoconjugate can contain from about 1.1 to about 50 molar equivalents of the adjuvant-linker compound with respect to the antibody.

The reactions can be conducted at any suitable temperature. In general, the reactions are conducted at a temperature of from about 4° C. to about 40° C. The reactions can be conducted, for example, at about 25° C. or about 37° C. The reactions can be conducted at any suitable pH. In general, the reactions are conducted at a pH of from about 4.5 to about 10. The reactions can be conducted, for example, at a pH of from about 5 to about 9. In some embodiments, the reaction is conducted at near neutral pH (i.e., around pH 7). In some embodiments, the reaction is conducted at a pH ranging from 7.2 to 7.5. The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 24 hours, or about 48 hours, or about 72 hours. Other reaction conditions may be employed in the methods of the invention, depending on the identity of the antibody in the immunoconjugate and the reagent used for installing the adjuvant moiety.

Reaction mixtures for forming the antibody adjuvant conjugates can contain additional reagents of the sort typically used in bioconjugation reactions. For example, in certain embodiments, the reaction mixtures can contain buffers (e.g., 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, tetrahydrofuran, acetone, and acetic acid), salts (e.g., NaCl, KCl, CaCl₂, and salts of Mn²⁺ and Mg²⁺), detergents/surfactants (e.g., a non-ionic surfactant such as N,N-bis[3-(D-gluconamido)propyl]cholamide, polyoxyethylene (20) cetyl ether, dimethyldecylphosphine oxide, branched octylphenoxy poly(ethyleneoxy)ethanol, a polyoxyethylene-polyoxypropylene block copolymer, t-octylphenoxypolyethoxyethanol, polyoxyethylene (20) sorbitan monooleate, and the like; an anionic surfactant such as sodium cholate, N-lauroylsarcosine, sodium dodecyl sulfate, and the like; a cationic surfactant such as hexdecyltrimethyl ammonium bromide, trimethyl(tetradecyl) ammonium bromide, and the like; or a zwitterionic surfactant such as an amidosulfobetaine, 3[(3-cholamidopropyl)dimethyl-ammonio]-1-propanesulfonate, and the like), chelators (e.g., ethylene glycol-bis(2-aminoethylether)-N,N,N′,N′-tetraacetic acid (EGTA), 2-({2-[bis(carboxymethyl)amino]ethyl} (carboxymethyl)amino)acetic acid (EDTA), and 1,2-bis(o-aminophenoxy)ethane-N,N,N′,N′-tetraacetic acid (BAPTA)), and reducing agents (e.g., dithiothreitol (DTT), β-mercaptoethanol (BME), and tris(2-carboxyethyl)phosphine (TCEP)). Buffers, cosolvents, salts, detergents/surfactants, chelators, and reducing agents can be used at any suitable concentration, which can be readily determined by those ordinarily skilled in the art. In general, buffers, cosolvents, salts, detergents/surfactants, chelators, and reducing agents are included in reaction mixtures at concentrations ranging from about 1 μM to about 1 M. For example, a buffer, a cosolvent, a salt, a detergent/surfactant, a chelator, or a reducing agent can be included in a reaction mixture at a concentration of about 1 μM, or about 10 μM, or about 100 μM, or about 1 mM, or about 10 mM, or about 25 mM, or about 50 mM, or about 100 mM, or about 250 mM, or about 500 mM, or about 1 M.

### Formulation and Administration of Immunoconjugates

In a related aspect, the invention provides a composition comprising a plurality of immunoconjugates as described above. In some embodiments, the average number of adjuvant moieties per immunoconjugate ranges from about 1 to about 10. The average number of adjuvant moieties per immunoconjugate can range, for example, from about 1 to about 10, or from about 1 to about 6, or from about 1 to about 4. The average number of adjuvant moieties per immunoconjugate can be about 0.8, 1, 1.2, 1.4, 1.6, 1.8, 2, 2.2, 2.4, 2.6, 2.8, 3, 3.2, 3.4, 3.6, 3.8, 4.0, or 4.2. In some embodiments, the average number of adjuvant moieties per immunoconjugate is about 4. In some embodiments, the average number of adjuvant moieties per immunoconjugate is about 2. In some cases, the antibody is covalently bonded to a single adjuvant moiety. In some cases, the antibody is covalently bonded to 2 or more adjuvant moieties (e.g., 3 or more, 4 or more, or 5 or more adjuvant moieties). In some cases, the antibody is covalently bonded to 1-10 adjuvant moieties (e.g., 1-8, 1-5, 1-3, 2-10, 2-8, 2-5, 2-3, or 3-8 adjuvant moieties). In some cases, the antibody is covalently bonded to 2-10 adjuvant moieties (e.g., 2-8, 2-5, 2-3, or 3-10, or 3-8 adjuvant moieties). In some cases in which the antibody is covalently bonded to more than one adjuvant moiety, the attached adjuvant moieties can be the same or different. For example, in some cases two or more of the adjuvant moieties can be the same (e.g., two different molecules of the same adjuvant moiety can each be attached to the antibody at a different site on the antibody). In some cases, the antibody is covalently bonded to 2 or more different adjuvant moieties (e.g., 3 or more, 4 or more, or 5 or more different adjuvant moieties). For example, when generating an immunoconjugate of the invention, one or more antibodies can be contacted with a mixture that includes two or more (e.g., 3 or more, 4 or more, or 5 or more) different adjuvant-linker compounds such that amino acid sidechains in the one or more antibodies reacts with the adjuvant-linker compounds, thus resulting in one or more immunoconjugates that are each covalently bonded to two or more different adjuvant moieties.

Site-specific antibody conjugation allows for precise placement of the adjuvant on the antibody and a homogenous DAR as compared to the heterogeneous conjugation product resulting from attachment to lysine residues in the antibody. Site-specific immunoconjugates may be generated through various modifications of the antibody. Methods for site-specific conjugation include the following methods but are not limited to those methods described herein. One method for site-specific conjugation involves the incorporation of a sequence that is then recognized by an enzyme, resulting in chemical modification. For example, the enzyme FGE recognizes the sequence Cys-X-Pro-X-Arg. Co-expression of the modified antibody along with FGE in mammalian culture generates an antibody containing an aldehyde-tag at the engineered site(s). Other enzymes may be used that recognize naturally occurring sequences or residues for conversion to chemically reactive groups allowing for site-specific conjugation. Bacterial transglutaminases (BTGs) can catalyze the formation of bonds between glutamine residues and primary amines; the bacterial enzyme sortase A can catalyze transpeptidation reactions through a recognition motif. Non-natural amino acids may also be incorporated into the antibody sequence that may then be reacted to generate site-specific conjugates. Naturally occurring residues, such as the amino acid selenocysteine, may be incorporated into the antibody and subsequently reacted with the appropriate reactive groups including but not limited to maleimides and iodoacetamides for site-specific conjugation. Another method is the incorporation of engineered cysteine residues that are added into the heavy or light chain of the antibody construct. Vectors encoding for the heavy and/or light chains are modified to incorporate the codon sequence for a cysteine residue. Conjugation is performed by first reducing the antibody and then re-oxidizing to regenerate the native disulfide bonds of the antibody, resulting in the uncapping of a reactive thiol(s). Once reacted with adjuvant-linker, the resulting product contains a homogenous population of immunoconjugate with a DAR defined by the number of cysteine residues engineered into the antibody. For example, the incorporation of a mutation in the light chain at position 205 from a valine to cysteine (V205C mutation) results in a product with the adjuvant conjugated at the defined sites (V205C).

In some embodiments, the composition further comprises one or more pharmaceutically acceptable excipients. For example, the immunoconjugates of the invention can be formulated for parenteral administration, such as intravenous (IV) administration or administration into a body cavity or lumen of an organ. Alternatively, the immunoconjugates can be injected intra-tumorally. Formulations for injection will commonly comprise a solution of the immunoconjugate dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic monoglycerides or diglycerides. In addition, fatty acids such as oleic acid can likewise be used in the preparation of injectables. These solutions are sterile and generally free of undesirable matter. These formulations can be sterilized by conventional, well known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the immunoconjugate in these formulations can vary widely, and will be selected primarily based on fluid volumes, viscosities, body weight, and the like, in accordance with the particular mode of administration selected and the patient's needs. In certain embodiments, the concentration of an immunoconjugate in a solution formulation for injection will range from about 0.1% (w/w) to about 10% (w/w).

In another aspect, the invention provides a method for treating cancer. The method includes comprising administering a therapeutically effective amount of an immunoconjugate (e.g., as a composition as described above) to a subject in need thereof. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also lie outside of these ranges, depending on the particular immunoconjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

Some embodiments of the invention provide methods for treating cancer as described above, wherein the cancer is a head and neck cancer. Head and neck cancer (as well as head and neck squamous cell carcinoma) refers to a variety of cancers characterized by squamous cell carcinomas of the oral cavity, pharynx and larynx, salivary glands, paranasal sinuses, and nasal cavity, as well as the lymph nodes of the upper part of the neck. Head and neck cancers account for approximately 3 to 5 percent of all cancers in the United States. These cancers are more common in men and in people over age 50. Tobacco (including smokeless tobacco) and alcohol use are the most important risk factors for head and neck cancers, particularly those of the oral cavity, oropharynx, hypopharynx and larynx. Eighty-five percent of head and neck cancers are linked to tobacco use. In the methods of the invention, the immunoconjugates can be used to target a number of malignant cells. For example, the immunoconjugates can be used to target squamous epithelial cells of the lip, oral cavity, pharynx, larynx, nasal cavity, or paranasal sinuses. The immunoconjugates can be used to target mucoepidermoid carcinoma cells, adenoid cystic carcinoma cells, adenocarcinoma cells, small-cell undifferentiated cancer cells, esthesioneuroblastoma cells, Hodgkin lymphoma cells, and Non-Hodgkin lymphoma cells. In some embodiments, methods for treating head and neck cancer include administering an immunoconjugate containing an antibody that is capable of binding EGFR (e.g., cetuximab, panitumumab, matuzumab, and zalutumumab), PD-1 (e.g., pembrolizumab), and/or MUC1.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer. In some embodiments, methods for treating breast cancer include administering an immunoconjugate containing an antibody that is capable of binding HER2 (e.g., trastuzumab, margetuximab), glycoprotein NMB (e.g., glembatumumab), and/or MUC1.

### Examples of Non-Limiting Aspects of the Disclosure

Aspects, including embodiments, of the present subject matter described herein may be beneficial alone or in combination, with one or more other aspects or embodiments. Without limiting the foregoing description, certain non-limiting aspects of the disclosure numbered 1-21 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. An immunoconjugate comprising (a) an antibody construct comprising (i) an antigen binding domain and (ii) an Fc domain, (b) an adjuvant moiety, and (c) a linker comprising an ethylene glycol group or a glycine residue, wherein each adjuvant moiety is covalently bonded to the antibody construct via the linker.

2. The immunoconjugate of aspect 1 wherein the antibody construct further comprises a targeting binding domain.

3. The immunoconjugate of aspect 1, wherein the antibody construct is an antibody.

4. The immunoconjugate of any one of aspects 1-3, wherein the antigen binding domain binds to an antigen of a cancer cell.

5. The immunoconjugate of any one of aspects 1-4, wherein the antigen binding domain binds to an antigen selected from the group consisting of CCR8, CDH1, CD19, CD20, CD29, CD30, CD38, CD40, CD47, EpCAM, MUC1, MUC16, EGFR, VEGF, HER2, SLAMF7, PDGFRa, and gp75.

6. The immunoconjugate of any one of aspects 3-5, wherein the antibody is an IgG1 antibody.

7. The immunoconjugate of any one of aspects 3-6, wherein the immunoconjugate has a structure according to Formula II:

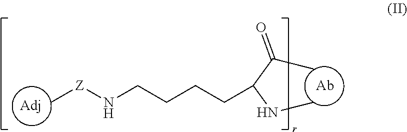

wherein

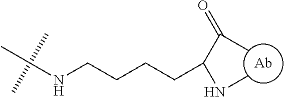

is an antibody with residue

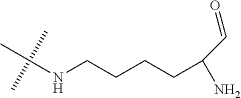

representing a lysine residue of the antibody, wherein “ [symbol] ” represents a point of attachment to Z; Adj is an adjuvant; subscript r is an integer from 1 to 10; and Z is a divalent linking moiety having an ethylene glycol group or a glycine residue.

8. The immunoconjugate of aspect 7, wherein Z comprises a poly(ethylene glycol) group.

9. The immunoconjugate of aspect 7 or 8, wherein Z comprises a glycine residue.

10. The immunoconjugate of any one of aspects 7-9, wherein Z further comprises a divalent cyclohexylene group.

11. The immunoconjugate of aspect 10, wherein the immunoconjugate has a structure according to Formula IIIa:

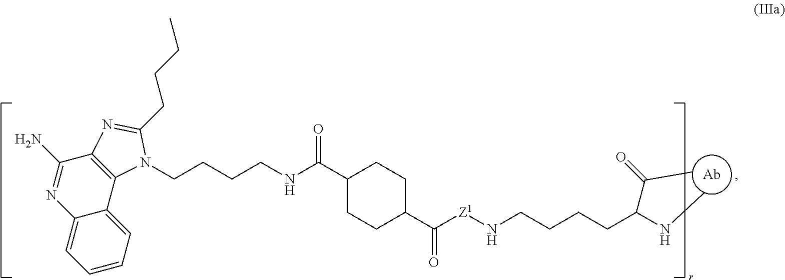

wherein Z¹ comprises at least one ethylene glycol group or at least one glycine residue.

12. The immunoconjugate of aspect 10, wherein the immunoconjugate has a structure according to Formula IIIb:

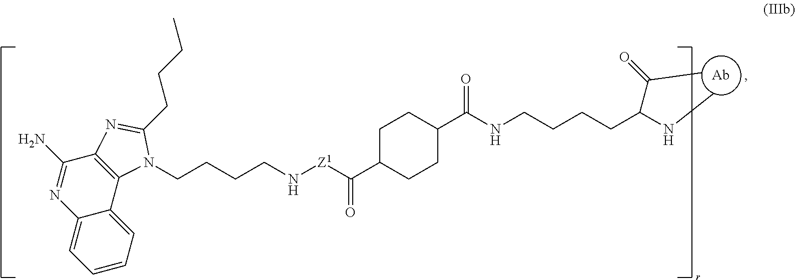

wherein Z¹ comprises at least one ethylene glycol group or at least one glycine residue.

13. The immunoconjugate of any one of aspects 7-9, wherein the immunoconjugate has a structure according to Formula IV:

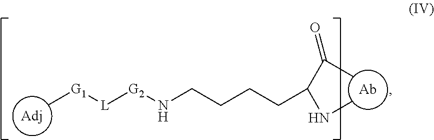

or a pharmaceutically acceptable salt thereof, wherein

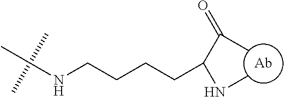

is an antibody with residue

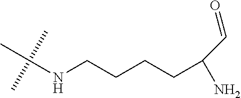

representing a lysine residue of the antibody, wherein “ [symbol] ” represents a point of attachment to G₂, Adj is an adjuvant, G₁ is CH₂, C═O, or a bond, G₂ is CH₂, C═O, or a bond, L is a linker, and subscript r is an integer from 1 to 10.

14. The immunoconjugate of aspect 13, wherein L is selected from the group consisting of:

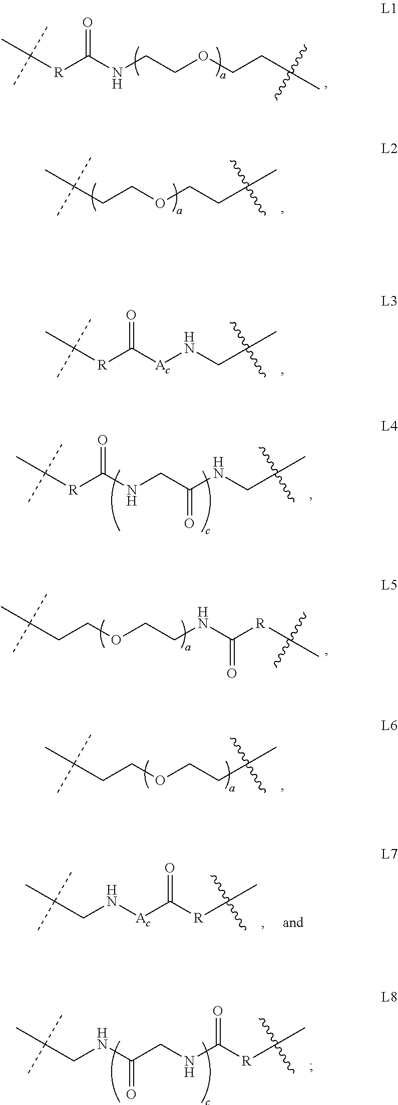

wherein R is optionally present and is a linear or branched, cyclic or straight, saturated or unsaturated alkyl, heteroalkyl, aryl, or heteroaryl chain comprising from 1 to 8 carbon units; a is an integer from 1 to 40; each A is independently selected from any amino acid; subscript c is an integer from 1 to 25; the dashed line (“ [symbol] ”) represents the point of attachment to G₁; and the wavy line (“ [symbol] ”) represents the point of attachment to G₂.

15. The immunoconjugate of aspect 13 or 14, wherein the immunoconjugate has a structure according to Formula IVa:

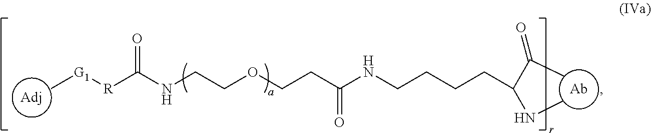

or a pharmaceutically acceptable salt thereof, wherein Ab is as defined herein; Adj is an adjuvant; G₁ is CH₂, C═O, or a bond; R is optionally present and is a linear or branched, cyclic or straight, saturated or unsaturated alkyl, heteroalkyl, aryl, or heteroaryl chain comprising from 1 to 8 carbon units; subscript a is an integer from 1 to 40; and subscript r is an integer from 1 to 10.

16. The immunoconjugate of aspect 13 or 14, wherein the immunoconjugate has a structure according to Formula IVb:

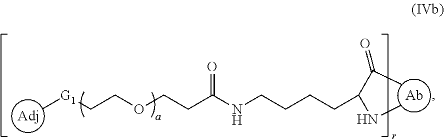

or a pharmaceutically acceptable salt thereof, wherein Ab is as defined herein; Adj is an adjuvant; G₁ is CH₂, C═O, or a bond; subscript a is an integer from 1 to 40; and subscript r is an integer from 1 to 10.

17. The immunoconjugate of aspect 13 or 14, wherein the immunoconjugates has a structure according to Formula IVc:

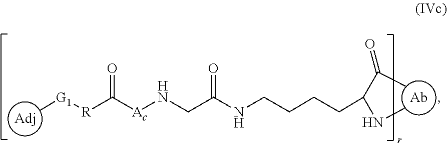

or a pharmaceutically acceptable salt thereof, wherein Ab is as defined herein; Adj is an adjuvant; G₁ is CH₂, C═O, or a bond; R is optionally present and is a linear or branched, cyclic or straight, saturated or unsaturated alkyl, heteroalkyl, aryl, or heteroaryl chain comprising from 1 to 8 carbon units; each A is independently selected from any amino acid; subscript c is an integer from 1 to 25; and subscript r is an integer from 1 to 10.

18. The immunoconjugate of aspect 17, wherein the immunoconjugate has a structure according to Formula IVd:

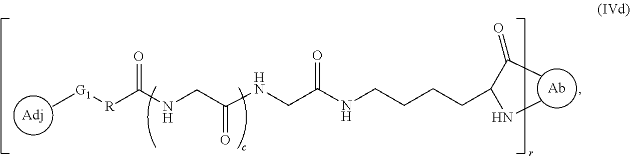

or a pharmaceutically acceptable salt thereof, wherein Ab is as defined herein; Adj is an adjuvant; G₁ is CH₂, C═O, or a bond; R is optionally present and is a linear or branched, cyclic or straight, saturated or unsaturated alkyl, heteroalkyl, aryl, or heteroaryl chain comprising from 1 to 8 carbon units; subscript c is an integer from 1 to 25; and subscript r is an integer from 1 to 10.

19. The immunoconjugate of aspect 13 or 14, wherein the immunoconjugate has a structure according to Formula IVe:

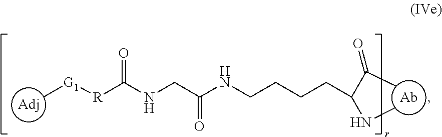

or a pharmaceutically acceptable salt thereof, wherein Ab is as defined herein; Adj is an adjuvant; G₁ is CH₂, C═O, or a bond; R is optionally present and is a linear or branched, cyclic or straight, saturated or unsaturated alkyl, heteroalkyl, aryl, or heteroaryl chain comprising from 1 to 8 carbon units; and subscript r is an integer from 1 to 10.

20. A composition comprising a plurality of immunoconjugates according to any one of aspects 1-19.

21. A method for treating cancer comprising administering a therapeutically effective amount of an immunoconjugate according to any one of aspects 1-19 or a composition according to aspect 20 to a subject in need thereof.

### EXAMPLES

### Example 1: Preparation of Immunoconjugates I-III

Imidazoquinoline 1 (1-(4-aminobutyl)-2-propyl-1H-imidazo[4,5-c]quinolin-4-amine) is reacted with 2,5-dioxopyrrolidin-1-yl 4-((2-((2-((2,5-dioxopyrrolidin-1-yl)oxy)-2-oxoethyl)amino)-2-oxoethyl)carbamoyl)cyclohexane-1-carboxylate to form NHS-Gly2-CC-1, shown in Scheme 1 below. Imidazoquinoline 1 is converted to NHS-EG-CC-1, shown in Scheme 2, in an analogous fashion. Imidazoquinoline 1 and aldehyde 2 are reacted in the presence of sodium borohydride, and the resulting intermediate is esterified with N-hydroxy succinimide to form NHS-EG-1, shown in Scheme 3.

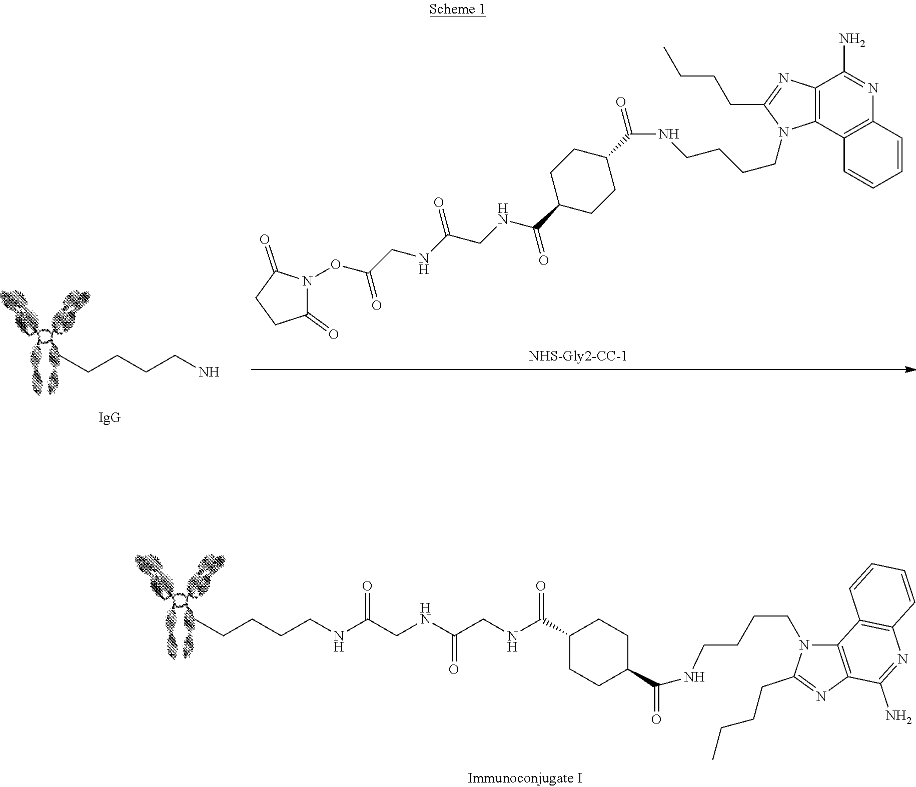

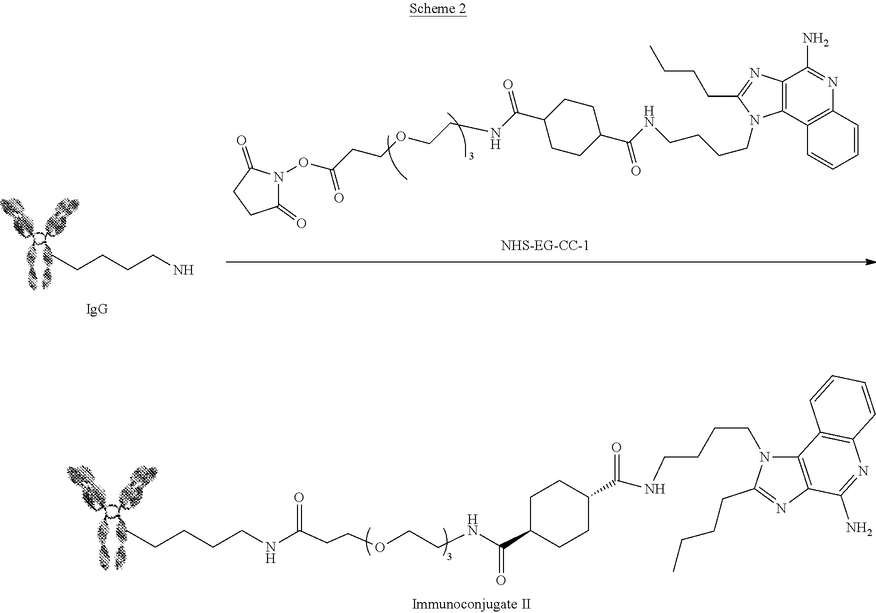

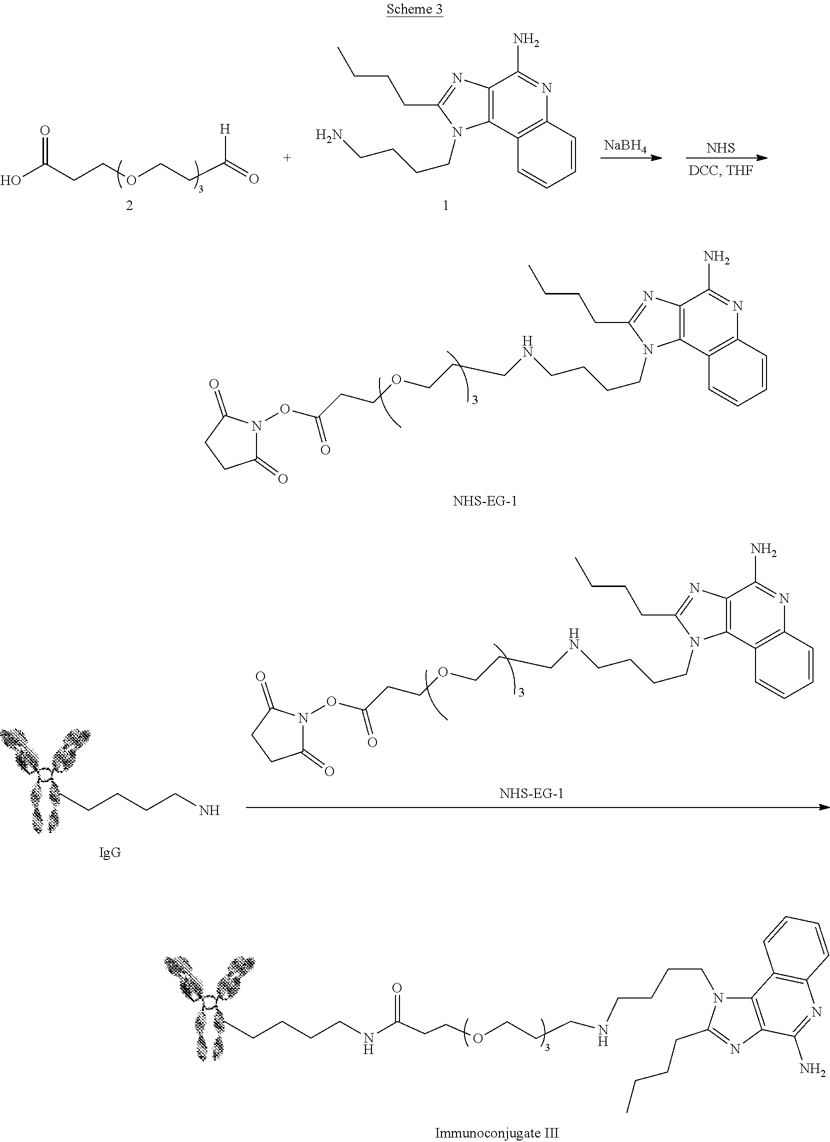

Antibody is resuspended in phosphate buffered saline (PBS) at 1-5 mg/mL is reacted with a 10-fold molar excess of NHS-Gly2-CC-1, NHS-EG-CC-1, or NHS-EG-1 at room temperature for 30 minutes. The resulting immunoconjugates are purified from excess reagent and byproducts with 3 washes in PBS with equilibrated Amicon Ultra Centrifugal Filter Units with Ultracel-100 membranes according to the manufacturer's instructions (EMD Millipore).

The average adjuvant to antibody ratio is determined via MALDI-TOF. Samples are desalted and buffer exchanged using Zeba Spin Desalting Columns (ThermoFisher Scientific) into deionized water. Matrix (sinapinic acid) is first spotted onto a MALDI sample target plate and allowed to dry. Next, the sample is mixed at a 1:1 ratio with and without a bovine serum albumin (BSA) standard (0.25-1 pM BSA) and spotted onto the plate with the matrix samples. Once both the matrix and sample layer are dry, samples are analyzed on a AB Sciex TOF/TOF 5800. A high mass detector (CovalX) with negative ionization allows for enhanced sensitivity and resolution at protein sizes in the range of a fully intact IgG antibody (˜150,000 kDa).

Human monocytes are observed to undergo DC differentiation following overnight stimulation with the immuoconjugates, whereas DC differentiation protocols with known stimulants (e.g., GM-CSF and IL-4) require longer activation periods.

### Example 2: Preparation of Immunoconjugate IV with a Pentafluorophenyl (“PFP”) Ester

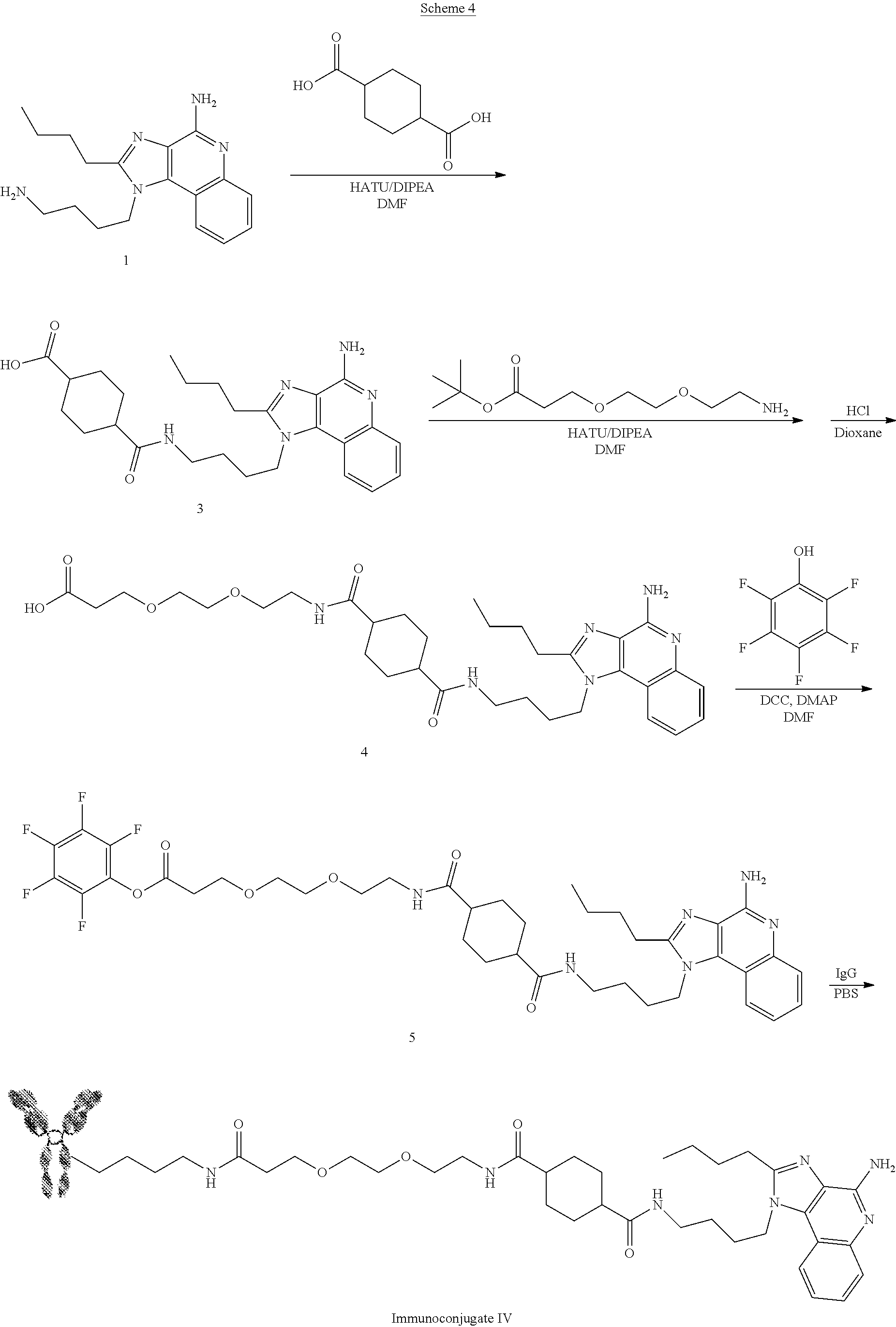

This example provides guidance on synthesis of an immunoconjugate using the PFP ester method. Ester modification of the adjuvant and conjugation of the modified adjuvant to the antibody is shown above in Scheme 4. Cyclohexane trans-1,4-dicarboxylate (1 g) was dissolved in 10 mL of dimethylformamide (“DMF”) and 14bis(dimethylamino)methylene]-1H-1,2,3-triazolo[4,5-b]pyridinium 3-oxid hexafluorophosphate) (“HATU”) (1 mmol) was added followed by 1 mL of N-ethyl-N-(propan-2-yl)propan-2-amine (“DIPEA”). Compound 1 (311 mg) was added and the mixture stirred overnight at 20° C. The reaction mixture was diluted with 50 mL of dichloromethane (“DCM”) and washed with 20 mL of 1N HCl. The DCM layer was evaporated to dryness and the product purified on silica gel eluted with 0-10% MeOH in DCM containing 1% acetic acid. Pure fractions were concentrated to provide 220 mg of purified acid 3. Compound 3 (100 mg) was dissolved in THF and 100 mg of HATU was added followed by 200 μL of DIPEA. Two equivalents of amino-PEG2-tertbutyl-carboxylate was added and stirred for one hour at 20° C. The mixture was concentrated to dryness and 10 milliliters of 4N HCl in dioxane was added. The mixture was concentrated to dryness and the crude product 4 was purified by prep HPLC to provide 40 mg of compound 4.

Compound 4 was converted to PFP ester 5 as described below. Compound 4 (35 mg) was added to 50 mg of PFP in 5 mL THF and 5 mL DMF was added followed by 20 mg of DCC. DMAP (2-3 mg) was added and the solution was stirred overnight at 20° C. The reaction was concentrated and purified by flash chromatography (eluted with 0-10% MeOH) to provide 17 mg of PFP ester 5 after lyophillization from 1:2 acetonitrile water.

PFP ester 5 (6 molar eq. relative to IgG) was added to 20 mg of an IgG antibody (specifically, the anti-CD20 antibody rituximab) (10 mg/mL in PBS) and incubated at 37° C. overnight. The resulting immunoconjugate IV was buffer exchanged into PBS (pH 7.2) to remove excess small molecular weight reagent and the concentration determined on the nanodrop. The yield was 15 mg of immunoconjugate IV (75% yield). The product was stored at 4° C. A DAR of 2.2 was determined via LC/MS analysis. Besides the desirable DAR and high yield, the product also had few impurities as determined by SEC analysis.

### Example 3: Preparation of Immunoconjugate V with a NHS Ester

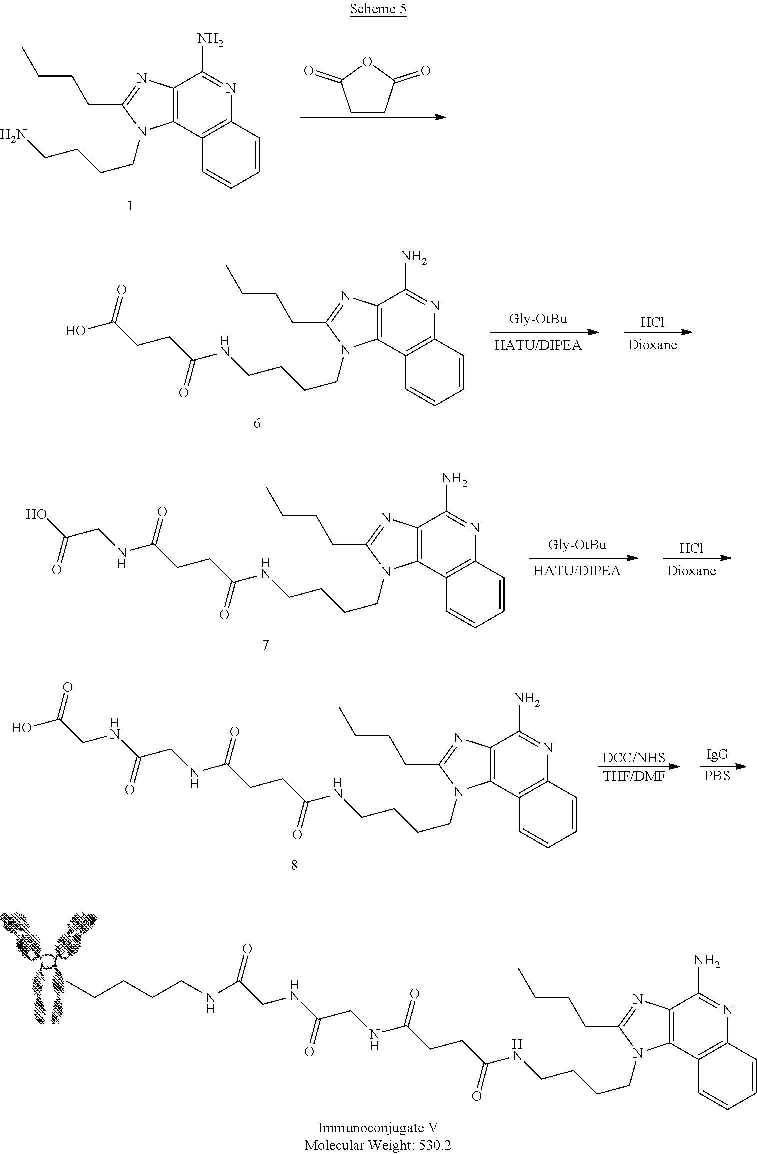

Ester modification of the adjuvant and conjugation of the modified adjuvant to the antibody is shown above in Scheme 5. Compound 1 (150 mg) was dissolved in 20 mL of tetrahydrofluran (“THF”) and 10 mL of aqueous, saturated sodium bicarbonate was added. Then, 50 mg of succinic anhydride was added in one portion and the mixture was stirred for one hour at room temperature. Twenty milliliters of 1N HCl was added slowly and the mixture was extracted with 2×50 mL of dichloromethane. The combined organic extracts were evaporated to dryness. The crude product (6) was purified on a 4 gram silica gel column eluted with 0-15% MeOH (1% acetic acid) over 15 minutes. Pure fractions were combined and evaporated to provide 190 mg of pure compound 6.

Compound 6 (150 mg) was dissolved in 10 mL of DMF and 1 equivalent of HATU was added followed by 2 equivalents of DIPEA. 1.5 equivalents of glycine-OtBu were added and stirred overnight. The DMF was evaporated and the residue treated with 5 mL of 1N HCl in dioxane for 30 minutes. The solvent was evaporated and the crude compound 7 was flash purified on a 4 gram silica gel column eluted with 0-10% MeOH over 10 minutes. Evaporation of pure fractions provided 110 mg of compound 7; the pure material was dissolved in DMF and the above process was repeated to provide 60 mg of pure compound 8.

The pure compound 8 (30 mg) was dissolved in 5 mL of DMF and 1.5 equivalents of NHS was added followed by 5 mL of THF. DCC (1.5 equivalents) was added and the mixture was stirred overnight at room temperature. The solvent was evaporated and the crude NHS ester was flash purified on a silica gel eluted with 0-10% MeOH in DCM over 10 minutes. Pure fractions (determined by TLC) were combined and evaporated to provide 1 mg of pure NHS-compound 8 after lyophilization from acetonitrile water.

The pure NHS ester was dissolved in DMSO to make a 20 mM solution and 6 eq. was added to 2 mL of an IgG antibody (specifically, the anti-CD20 antibody rituximab) (10 mg/mL in PBS). The conjugation reaction was incubated at room temperature overnight and buffer exchanged into fresh PBS to remove excess adjuvant. The purified immunoconjugate V was sterile filtered and stored at 4° C. The yield was about 16 mg. Besides having a high yield, the LC/MS analysis showed high levels of purity, low levels of aggregation, and a desirable DAR ratio.

### Example 4: Preparation of Immunoconjugate VI with a TFP Ester

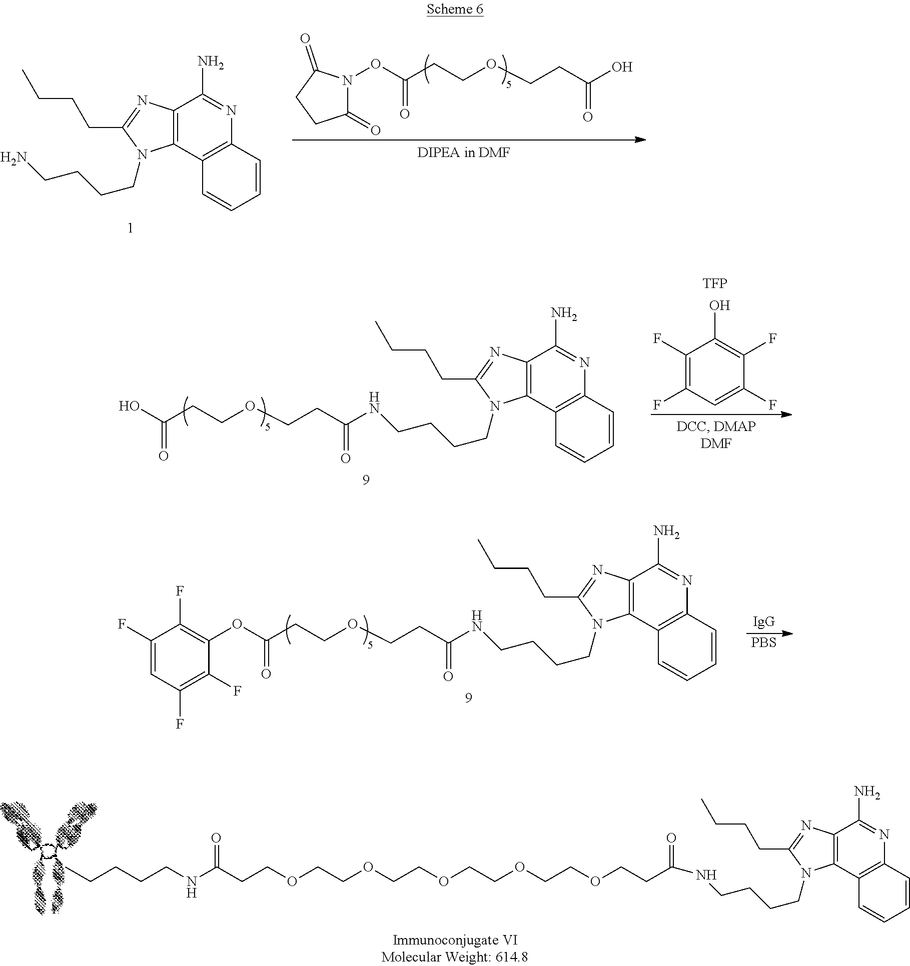

This example provides guidance on synthesis of an immunoconjugate with a different linker using the TFP ester method. Ester modification of the adjuvant and conjugation of the modified adjuvant to the antibody is shown above in Scheme 6. Compound 1 (311 mg, 1 mmol) was dissolved in 10 mL of DMF and then 0.3 mL of DIPEA was added. The NHS-PEGS-acid (1.2 equivalents) was dissolved in 5 mL of dichloromethane and added to compound 1 in one portion. The mixture was stirred overnight at room temperature and then concentrated to dryness. The crude residue was purified via silica gel chromatography on a 4 gram column eluted with 0-10% MeOH in DCM containing 1% acetic acid over 10 minutes to provide 260 mg (57% yield) of compound 9 after concentration of the pure fractions.

Compound 9 (50 mg) was dissolved in 10 mL DMF and 1.5 eq. of TFP was added followed by 1.2 eq. DCC and 5 mg of DMAP. The reaction was stirred overnight, concentrated to dryness and purified on silica gel 4 gram column eluted with 0-10% MeOH in DCM to provide 35 mg of pure Compound 10 after lyophilization from 1:2 acetonitrile water.

The TFP ester (10) was dissolved in DMSO to make a 20 mM stock solution and added to 20 mg of an IgG antibody (specifically, the anti-CD20 antibody rituximab) in PBS at 10 mg/mL. The conjugation reaction was allowed to proceed overnight at room temperature. The resulting immunoconjugate VI was buffer exchanged (GE, PD10 desalting column) into PBS at pH 7.4. The purified immunoconjugate was sterile filtered using a 2 μm syringe filter and stored at 4° C. LC/MS analysis confirmed that the process provided a DAR of 2.9 adjuvants per antibody. SEC analysis indicated minimal amounts of aggregate (i.e., less than 2%).

### Example 5: Preparation of Immunoconjugate VIII with a TFP Ester

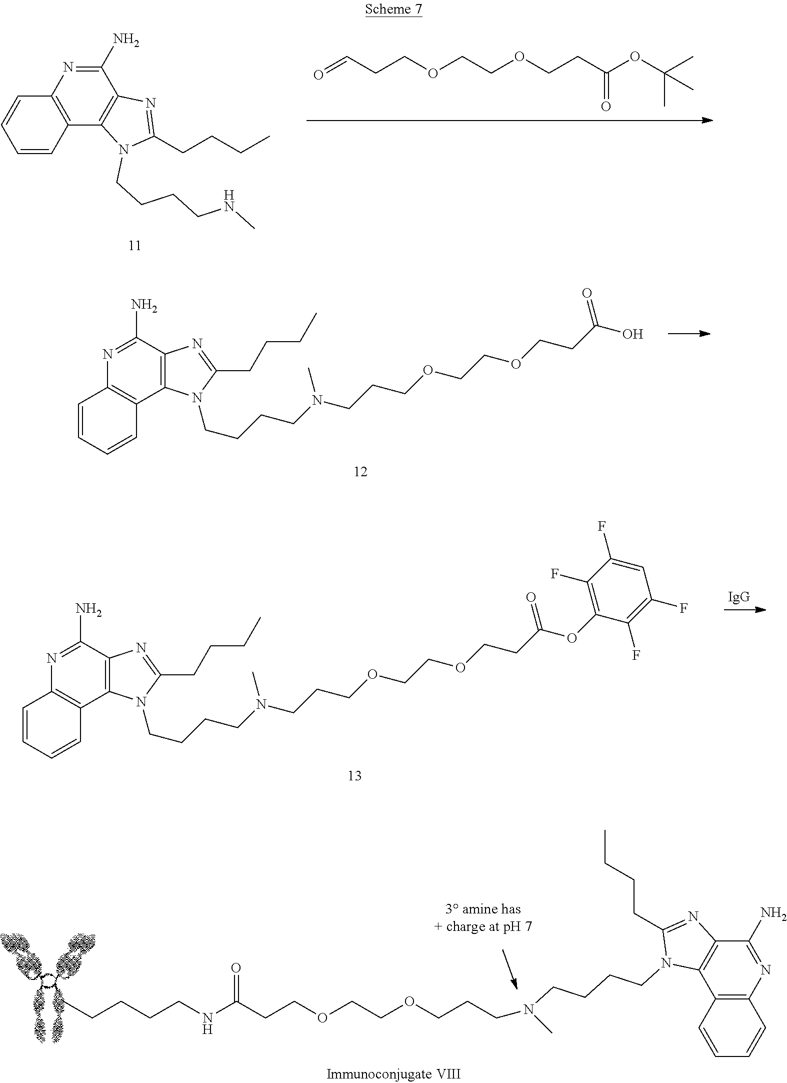 [SMILES: CCCCc1nc2c(N)nc3ccccc3c2n1CCCCNC ; CC(C)(C)OC(=O)CCOCCOCCC=O ; CCCCc1nc2c(N)nc3ccccc3c2n1CCCCN(C)CCCOCCOCCC(=O)O ; CCCCc1nc2c(N)nc3ccccc3c2n1CCCCN(C)CCCOCCOCCC(=O)Oc1c(F)c(F)cc(F)c1F ; CCCCCNC(=O)CCOCCOCCCN(C)CCCCn1c(CCCC)nc2c(N)nc3ccccc3c21]

This example provides guidance on synthesis of an immunoconjugate that contains a PEG tertiary amine linker using the TFP method. Compound 11 (200 mg) was dissolved in methanol (20 mL) and 3 eq. of tert-butyl 3-(2-(3-oxopropoxy)ethoxy)propanoate was added followed by 1.1 equivalents of NaCNBH₄. The mixture was stirred for 3 hours at room temperature and concentrated to dryness. Trifluoroacetic acid (TFA, 10 mL) was added and the reaction stirred for 2 hours at room temperature. The TFA was evaporated under vacuum and the crude product was purified by preparative HPLC on a C-18 column. The product was eluted with a gradient of 10-90% acetonitrile in water (0.1% TFA) over 20 minutes to provide 85 mg of purified acid 12 after lyophilization of the combined pure fractions (confirmed by LC/MS).

Compound 12 (80 mg) was dissolved in dichloromethane/dimethylformamide (5 mL, 1:1) and 2 equivalents of TFP was added followed by 1.2 equivalents of EDCI. The reaction was stirred overnight at room temperature. The crude TFP ester product 13 was purified via flash chromatography on a 4 gram silica gel column eluted with 0-10% isopropanol over 10 minutes. Pure fractions were concentrated and the residue lyophilized from 30% acetonitrile water to provide 45 mg of purified TFP ester of compound 13 as a beige solid. The molecular weight and purity were confirmed by LC/MS (m/z=647.7).

Conjugation to antibody: The TFP ester of compound 13 was dissolved in anhydrous DMSO to make a 20 mM stock solution and 8 molar equivalents (relative to the antibody) was added to an IgG1 antibody (specifically, the anti-CD20 antibody rituxumab) (10 mg/mL in PBS). The conjugation reaction was incubated at 4° C. overnight. The resulting immunoconjugate VIII was buffer exchanged into PBS (pH 7.2) to remove excess small molecular weight reagents. The final concentration was determined by measuring the antibodies at 280 nm on the Nanodrop 1000 spectrophotometer. The yield was 15 mg of immunoconjugate VIII (75%) which was stored at 4° C. until used.

Minimal aggregate was seen (less than 1%) as detected by SEC analysis. The product had a DAR ratio of 2.2 as determined via LC/MS analysis. The purified immunoconjugate VIII was filtered through a 0.2 μM sterile filter and stored at −20° C.

### Example 6: Preparation of Immunoconjugate IX with a TFP Ester

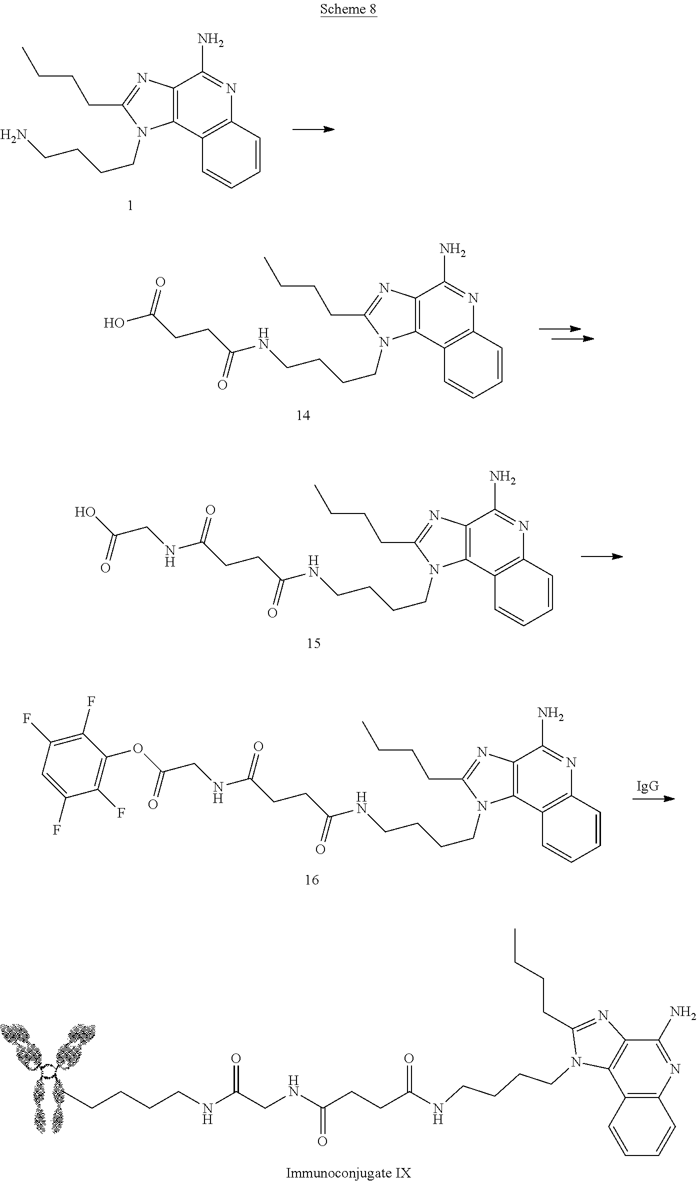 [SMILES: CCCCc1nc2c(N)nc3ccccc3c2n1CCCCN ; CCCCc1nc2c(N)nc3ccccc3c2n1CCCCNC(=O)CCC(=O)O ; CCCCc1nc2c(N)nc3ccccc3c2n1CCCCNC(=O)CCC(=O)NCC(=O)O ; CCCCc1nc2c(N)nc3ccccc3c2n1CCCCNC(=O)CCC(=O)NCC(=O)Oc1c(F)c(F)cc(F)c1F ; CCCCCNC(=O)CNC(=O)CCC(=O)NCCCCn1c(CCCC)nc2c(N)nc3ccccc3c21]

This example provides guidance on synthesis of an immunoconjugate with a different linker using the TFP ester method. Compound 1 (150 mg) was dissolved in 20 mL THF and 10 mL of aqueous saturated sodium bicarbonate was added. Succinic anhydride (50 mg) was added in one portion and the mixture stirred for 1 hour at room temperature. 20 mL of 1N HCl was added slowly and the mixture was extracted with 2×50 mL of dichloromethane and the combined organic extracts were evaporated to dryness. The crude product 14 was purified on a 4 gram silica gel column eluted with 0-15% MeOH (1% acetic acid) over 15 minutes. Pure fractions were combined and evaporated to provide 180 mg of pure compound 14.

One hundred and fifty mg of compound 14 was dissolved in DMF (10 mL) and 1 equivalent of HATU was added followed by 2 equivalents of DIPEA. One and a half eq. of glycine-OtBu was added and stirred overnight. The DMF was evaporated and the residue treated with 5 mL of 1N HCl in dioxane for 30 minutes with stirring. The solvent was evaporated and the crude residue was flash purified on a 4 gram silica gel column eluted with 0-10% isopropanol over 15 minutes. Evaporation of pure fractions provided 110 mg of pure 15.

Compound 15 (50 mg) was dissolved in 10 mL DMF and 1.5 eq. of TFP was added followed by 1.2 eq. DCC and 2 mg of DMAP. The reaction was stirred overnight, concentrated to dryness and purified on silica gel (4g column) eluted with 0-10% IPA in DCM to provide 32 mg of pure TFP ester, compound 16, after lyophilization from 1:3 acetonitrile water.

Conjugation to antibody: The TFP ester, compound 16, was dissolved in anhydrous DMSO to make a 20 mM stock solution and 5 molar equivalents (relative to the antibody) was added to 20 mg antibody at 10 mg/mL in PBS. The conjugation reaction was incubated at 4° C. for 6 hours. The resulting immunoconjugate IX was buffer exchanged into PBS (pH 7.4) to remove excess small molecular weight impurities. The final protein concentration was determined by measuring the absorbance at 280 nm on a Nanodrop 1000 spectrophotometer. The yield was 15 mg (75% based on recovered protein). SEC analysis detected minimal aggregate of less than 1% and the DAR was determined to be 2.8 adjuvants per antibody via LC/MS analysis. The purified immunoconjugate was filtered through a 0.2 μM sterile filter and stored at −20° C. until needed.

### Example 7: Preparation of Immunoconjugate Xa TFP Ester

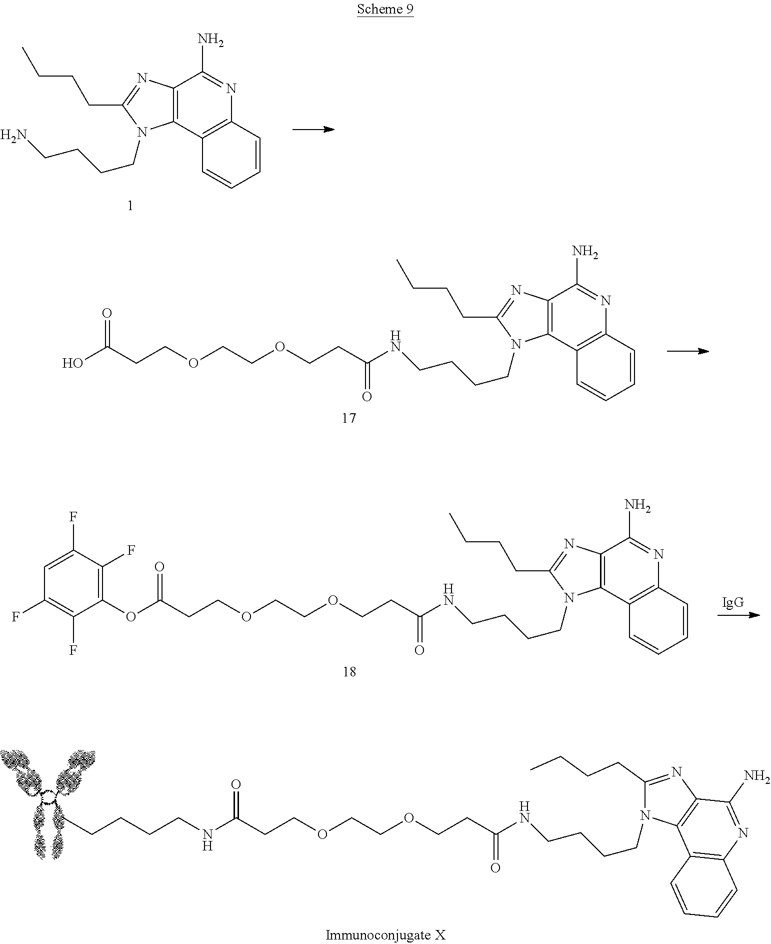 [SMILES: CCCCc1nc2c(N)nc3ccccc3c2n1CCCCN ; CCCCc1nc2c(N)nc3ccccc3c2n1CCCCNC(=O)CCOCCOCCC(=O)O ; CCCCc1nc2c(N)nc3ccccc3c2n1CCCCNC(=O)CCOCCOCCC(=O)Oc1c(F)c(F)cc(F)c1F ; CCCCCNC(=O)CCOCCOCCC(=O)NCCCCn1c(CCCC)nc2c(N)nc3ccccc3c21]

This example provides guidance on synthesis of an immunoconjugate with a different linker using the TFP method. Compound 1 (155 mg, 0.5 mmol) was dissolved in 10 mL of DMF and 0.2 mL of DIPEA was added. In a separate container, 1.2 equivalents of PEG2-dicarboxylate mono methyl ester was dissolved in 5 mL of DMF and 2 equivalents DIPEA was added followed by HATU (1.2 equivalents). The mixture was added to 1 and stirred 1 hour at room temperature. The reaction was concentrated to dryness under vacuum and the residue was dissolved in THF (5 mL). An equal volume of water was added followed by 2 mL of 1 M aqueous LiOH. The mixture was stirred overnight and then 10 mL of 1N HCl was added. The acidified mixture was extracted 2× with dichloromethane, dried over sodium sulfate, concentrated to dryness and purified via silica gel chromatography. The product was eluted with 0-10% methanol over 10 minutes. The pure fractions were combined and concentrated to provide 110 mg of pure compound 17 as a pale yellow solid.

Compound 17 (50 mg) was dissolved in dichloromethane/dimethylformamide (5 mL, 1:1) and 2 equivalents of TFP was added followed by 1.5 equivalents of EDCI. The reaction was stirred overnight at ambient temperature and the reaction was concentrated to dryness. The crude TFP ester 18 was purified via flash chromatography on a 4 gram silica gel column eluted with 0-10% isopropanol over 10 minutes. Pure fractions were concentrated and the residue was lyophilized from 30% acetonitrile in water to provide 41 mg of purified TFP ester 18 as a white solid. The molecular weight and purity were confirmed by LC/MS.

Conjugation to antibody: The TFP ester 18 was dissolved in anhydrous DMSO to make a 20 mM stock solution and 8 molar equivalents (relative to the antibody) was added to 20 mL of an IgG antibody (specifically, the anti-CD20 antibody rituximab) (10 mg/mL in PBS). The conjugation reaction was incubated at 4° C. overnight. The resulting immunoconjugate X was buffer exchanged into PBS (pH 7.2) to remove excess small molecular weight impurities. The final concentration was determined by measuring the absorbance at 280 nm on a Thermo Nanodrop 1000 spectrophotometer. The yield was 16 mg of conjugated immunoconjugate X, or 70% based on recovered protein. Minimal aggregate (less than 1%) was detected by SEC analysis and a DAR of 2.3 was determined via LC/MS analysis. The purified immunoconjugate was filtered through a 0.2 μM sterile filter and stored at −20° C.

### Example 8: Preparation of Immunoconjugates XI and XII with a TFP Ester

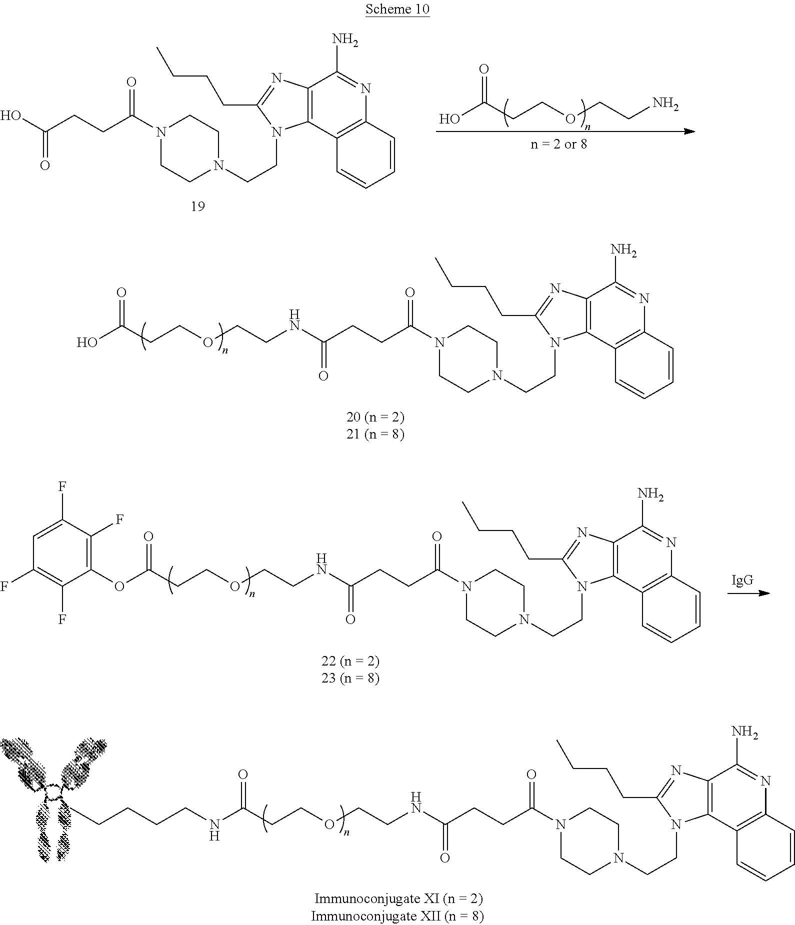

This example provides guidance on synthesis of immunoconjugates with different linkers using the TFP ester method. Compound 19 (Scheme 10) was coupled to polyethylene glycol (PEG) linkers containing 2 or 8 PEG. units in order to extend the distance between the adjuvant and the antibody. Attachment of the PEG linker extensions was performed using previously described protocols for linker attachment and TFP activation. Briefly 100 mg of compound 19 was dissolved in 10 mL of DMF and 0.2 mL of DIPEA was added followed by HATU (1.2 equivalents). After 1 hour the appropriate amino PEG linker (n=2 or 8) was added and stirred an additional 2 hours at room temperature. The reaction mixture was concentrated to dryness under vacuum and the residue was purified via preparative HPLC on a C-18 column eluted with 10-90% acetonitrile in water over 30 minutes. The pure fractions were combined and lyophilized to provide 65 mg and 45 mg of intermediates 20 or 21 as a clear glassy substance.

Compounds 20 and 21 were converted to the corresponding TFP esters 22 and 23 using previously described protocols. Briefly, the free acid 20 or 21 (50 mg) was dissolved in dichloromethane/dimethylformamide (5 mL, 1:1) and 2 equivalents of TFP was added followed by 1.5 equivalents of EDCI. The mixture was stirred overnight at room temperature and concentrated to dryness to provide crude TFP esters 22 and 23. The crude TFP esters were purified via flash chromatography on silica gel and eluted with 0-10% isopropanol over 10 minutes. Pure fractions were concentrated and the residue was lyophilized from 30% acetonitrile in water to provide purified TFP esters 22 and 23 as clear solids. The molecular weight and purity of the pure compounds were confirmed by LC/MS.

Conjugation to antibody: TFP esters 22 and 23 were conjugated to an IgG1 antibody (specifically, the anti-CD20 antibody rituxumab) using previously described protocols. The TFP esters were dissolved in anhydrous DMSO to make a 20 mM stock solution and 8 molar equivalents (relative to the antibody) was added to 20 mg of the IgG antibody at 10 mg/mL in PBS. The conjugation reaction was incubated at 4° C. for 12 hours. The resulting immunoconjugates, XI and XII were buffer exchanged into PBS (pH 7.4) to remove excess small molecular weight impurities. The final protein concentration was determined by measuring the absorbance at 280 nm on a Nanodrop 1000 spectrophotometer. The yields were 75% based on recovered protein. SEC analysis detected minimal aggregate was present and the DARs of 1.0 and 1.7 adjuvants per antibody were determined via LC/MS analysis. The purified immunoconjugates were filtered through a 0.2 μM sterile filter and stored at −20° C. until needed.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An immunoconjugate comprising
(a) an antibody construct comprising (i) an antigen binding domain and (ii) an Fc domain,
(b) an adjuvant moiety of formula: 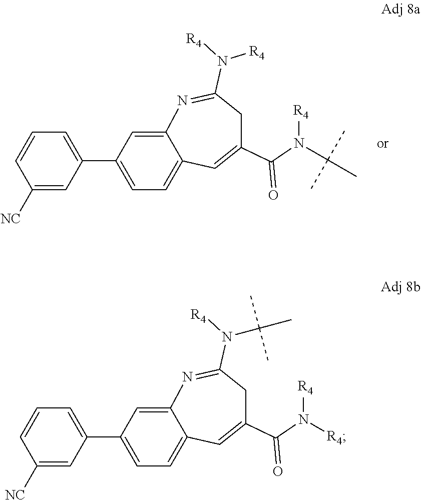
wherein each R⁴ independently is hydrogen, or an alkyl, heteroalkyl, cycloalkyl, heterocycloalkyl, aryl, heteroaryl, arylalkyl, or heteroarylalkyl group comprising from 1 to 8 carbons and the dashed line (“ [symbol] ”) represents the point of attachment of the adjuvant, and (c) a linker,
wherein each adjuvant moiety is covalently bonded to the antibody construct via the linker.

2. The immunoconjugate of claim 1, wherein the antigen binding domain binds to an antigen selected from the group consisting of CDH1, CD19, CD20, CD29, CD30, CD38, CD40, CD47, EpCAM, MUC1, MUC16, EGFR, VEGF, HER2, SLAMF7, PDGFRa, gp75, CTLA4, PD-1, PD-L1, PD-L2, LAG-3, B7-H4, KIR, TNFRSF4, OX40L, IDO-2, CEACAM1, BTLA, TIM3, A2Ar, VISTA, CLEC4C (BDCA-2, DLEC, CD303, CLECSF7), CLEC4D (MCL, CLECSF8), CLEC4E (Mincle), CLEC6A (Dectin-2), CLEC5A (MDL-1, CLECSF5), CLEC1B (CLEC-2), CLEC9A (DNGR-1), and CLEC7A (Dectin-1).

3. The immunoconjugate of claim 1, wherein the antigen binding domain binds to EGFR, HER2, or PD-L1.

4. The immunoconjugate of claim 1, wherein the antibody construct is an antibody.

5. The immunoconjugate of claim 4, wherein the antibody is selected from the group consisting of pembrolizumab, nivolumab, atezolizumab, avelumab, ipilimumab, obinutuzumab, trastuzumab, cetuximab, rituximab, pertuzumab, bevacizumab, daratumumab, etanercept, olaratumab, elotuzumab, margetuximab, and a biosimilar thereof.

6. The immunoconjugate of claim 4, wherein the antibody comprises a modified Fc region.

7. The immunoconjugate of claim 4, wherein the immunoconjugate has a structure according to Formula II: 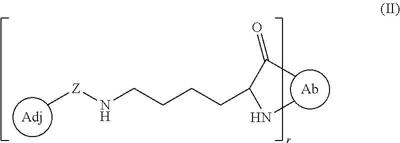
wherein 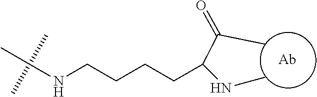
is an antibody with residue 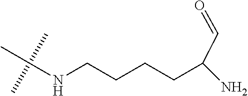
representing a lysine residue of the antibody, wherein “ [symbol] ” represents a point of attachment to Z, Adj is an adjuvant, subscript r is an integer from 1 to 10, and Z is a divalent linking moiety comprising an ethylene glycol group or a glycine residue.

8. The immunoconjugate of claim 4, wherein the immunoconjugate has a structure according to Formula IV: 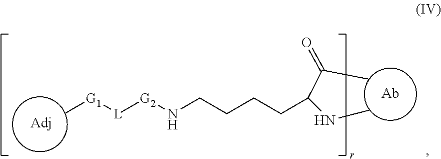
or a pharmaceutically acceptable salt thereof, wherein is an 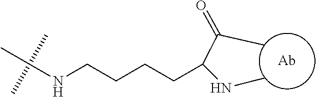
antibody with residue 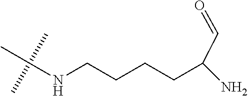
representing a lysine residue of the antibody, wherein “ [symbol] ” represents a point of attachment to G₂, Adj is the adjuvant, G₁ is CH₂, C═O, or a bond, G₂ is CH₂, C═O, or a bond, L is a linker, and subscript r is an integer from 1 to 10.

9. The immunoconjugate of claim 8, wherein L is selected from: 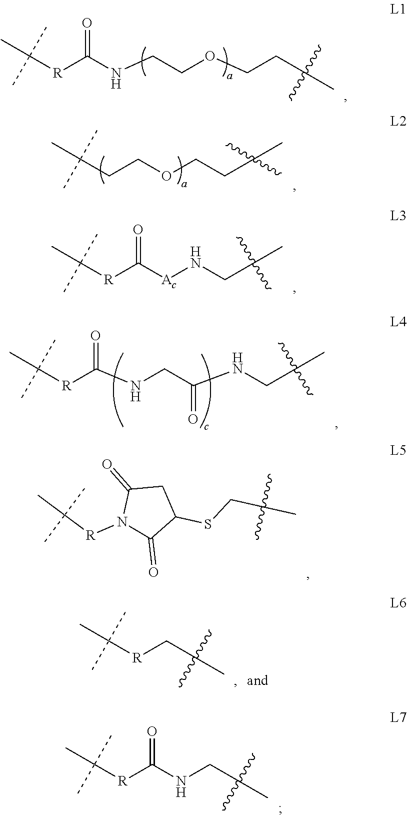
wherein R is optionally present and is a linear or branched, cyclic or straight, saturated or unsaturated alkyl, heteroalkyl, aryl, or heteroaryl chain comprising from 1 to 8 carbons, a is an integer from 1 to 40, each A is independently selected from any amino acid; subscript c is an integer from 1 to 25, the dashed line (“ [symbol] ”) represents the point of attachment to G₁, and the wavy line (“ [symbol] ”) represents the point of attachment to G₂.

10. The immunoconjugate of claim 1, wherein the adjuvant moiety is: 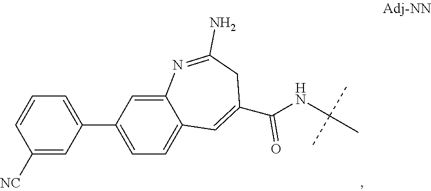
wherein the dashed line (“ [symbol] ”) represents the point of attachment of the adjuvant.

11. An immunoconjugate comprising
(a) an antibody construct comprising (i) an antigen binding domain and (ii) an Fc domain,
(b) an adjuvant moiety of formula: 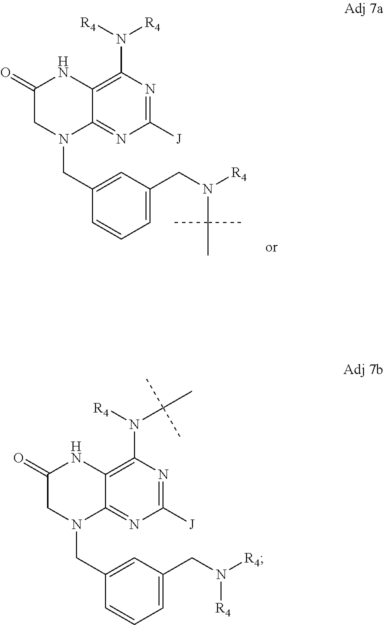
wherein J is hydrogen, OR⁴, or R⁴, each R⁴ independently is hydrogen, or an alkyl, heteroalkyl, cycloalkyl, heterocycloalkyl, aryl, heteroaryl, arylalkyl, or heteroarylalkyl group comprising from 1 to 8 carbons, and the dashed line (“ [symbol] ”) represents the point of attachment of the adjuvant, and
(c) a linker,
wherein each adjuvant moiety is covalently bonded to the antibody construct via the linker.

12. The immunoconjugate of claim 11, wherein the antigen binding domain binds to an antigen selected from the group consisting of CDH1, CD19, CD20, CD29, CD30, CD38, CD40, CD47, EpCAM, MUC1, MUC16, EGFR, VEGF, HER2, SLAMF7, PDGFRa, gp75, CTLA4, PD-1, PD-L1, PD-L2, LAG-3, B7-H4, KIR, TNFRSF4, OX40L, IDO-1, IDO-2, CEACAM1, BTLA, TIM3, A2Ar, VISTA, CLEC4C (BDCA-2, DLEC, CD303, CLECSF7), CLEC4D (MCL, CLECSF8), CLEC4E (Mincle), CLEC6A (Dectin-2), CLEC5A (MDL-1, CLECSF5), CLEC1B (CLEC-2), CLEC9A (DNGR-1), and CLEC7A (Dectin-1).

13. The immunoconjugate of claim 11, wherein the antigen binding domain binds to EGFR, HER2, or PD-L1.

14. The immunoconjugate of claim 11, wherein the antibody construct is an antibody.

15. The immunoconjugate of claim 14, wherein the antibody is selected from the group consisting of pembrolizumab, nivolumab, atezolizumab, avelumab, ipilimumab, obinutuzumab, trastuzumab, cetuximab, rituximab, pertuzumab, bevacizumab, daratumumab, etanercept, olaratumab, elotuzumab, margetuximab, and a biosimilar thereof.

16. The immunoconjugate of claim 14, wherein the antibody comprises a modified Fc region.

17. The immunoconjugate of claim 14, wherein the immunoconjugate has a structure according to Formula II: 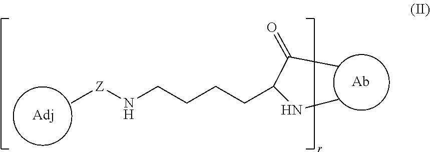
wherein 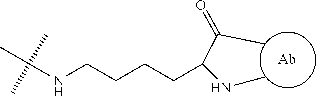
is an antibody with residue 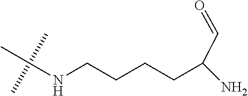
representing a lysine residue of the antibody, wherein “ [symbol] ” represents a point of attachment to Z, Adj is an adjuvant, subscript r is an integer from 1 to 10, and Z is a divalent linking moiety comprising an ethylene glycol group or a glycine residue.

18. The immunoconjugate of claim 14, wherein the immunoconjugate has a structure according to Formula IV: 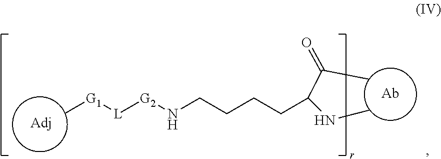
or a pharmaceutically acceptable salt thereof, wherein is an 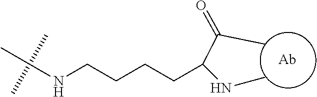
antibody with residue 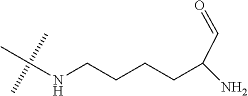
representing a lysine residue of the antibody, wherein “ [symbol] ” represents a point of attachment to G₂, Adj is the adjuvant, G₁ is CH₂, C═O, or a bond, G₂ is CH₂, C═O, or a bond, L is a linker, and subscript r is an integer from 1 to 10.

19. The immunoconjugate of claim 18, wherein L is selected from: 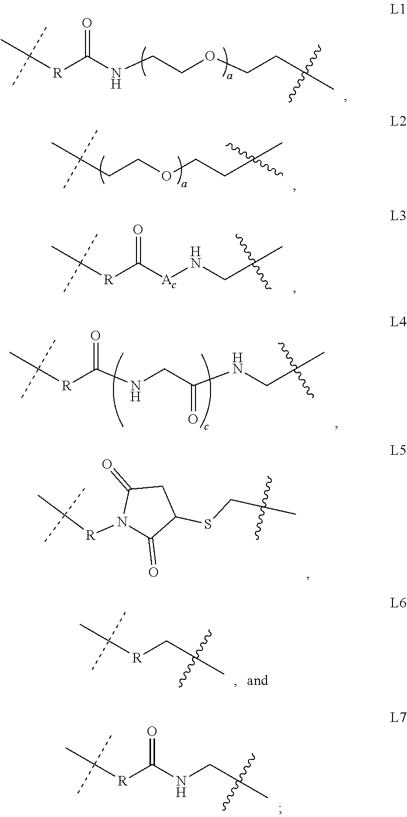
wherein R is optionally present and is a linear or branched, cyclic or straight, saturated or unsaturated alkyl, heteroalkyl, aryl, or heteroaryl chain comprising from 1 to 8 carbons, a is an integer from 1 to 40, each A is independently selected from any amino acid; subscript c is an integer from 1 to 25, the dashed line (“ [symbol] ”) represents the point of attachment to G₁, and the wavy line (“ [symbol] ”) represents the point of attachment to G₂.

20. The immunoconjugate of claim 11, wherein the adjuvant moiety is: 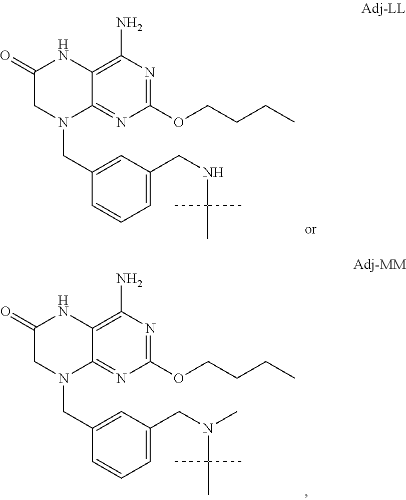
wherein the dashed line (“ [symbol] ”) represents the point of attachment of the adjuvant.
